SEQUENCE LISTING (33 sequences)

SEQ ID NO: 1 - protein, 29 aa, Artificial sequence
  Synthetic Peptide
  MOD_RES (2)..(2): Aib
  SITE (16)..(16): Glu side chain lactam linkage to Lys
  SITE (16)..(20): cyclic
  MOD_RES (17)..(17): Z17CO2H
  SITE (20)..(20): Lys side chain lactam linkage to Glu
HXQGTFTSDYSKYLDEKAAKEFIQWLLQT

SEQ ID NO: 2 - protein, 29 aa, Artificial sequence
  Synthetic Peptide
  MOD_RES (2)..(2): Aib
  SITE (16)..(16): Glu side chain lactam linkage to Lys
  SITE (16)..(20): cyclic
  MOD_RES (17)..(17): Me15CO2H
  SITE (20)..(20): Lys side chain lactam linkage to Glu
HXQGTFTSDYSKYLDEKAAKEFIQWLLQT

SEQ ID NO: 3 - protein, 29 aa, Artificial sequence
  Synthetic Peptide
  MOD_RES (2)..(2): Aib
  SITE (16)..(16): Glu side chain lactam linkage to Lys
  SITE (16)..(20): cyclic
  SITE (20)..(20): Lys side chain lactam linkage to Glu
  MOD_RES (24)..(24): Z15CO2H
HXQGTFTSDYSKYLDEQAAKEFIKWLLQT

SEQ ID NO: 4 - protein, 29 aa, Artificial sequence
  Synthetic Peptide
  MOD_RES (2)..(2): Aib
  SITE (16)..(16): Glu side chain lactam linkage to Lys
  SITE (16)..(20): cyclic
  SITE (20)..(20): Lys side chain lactam linkage to Glu
  MOD_RES (24)..(24): Z17CO2H
HXQGTFTSDYSKYLDEQAAKEFIKWLLQT

SEQ ID NO: 5 - protein, 29 aa, Artificial sequence
  Synthetic Peptide
  MOD_RES (2)..(2): Aib
  SITE (16)..(16): Glu side chain lactam linkage to Lys
  SITE (16)..(20): cyclic
  MOD_RES (17)..(17): Z17CO2H
  SITE (20)..(20): Lys side chain lactam linkage to Glu
HXQGTFTSDYSRYLDEKAAKEFIQWLLQT

SEQ ID NO: 6 - protein, 29 aa, Artificial sequence
  Synthetic Peptide
  MOD_RES (2)..(2): Aib
  SITE (16)..(20): cyclic
  SITE (16)..(16): Glu side chain lactam linkage to Lys
  MOD_RES (17)..(17): Lys(N-omega(1-(17-carboxyl-heptadecyloxy) beta-D-glucuronyl)) or Lys(Z17CO2H)
  SITE (20)..(20): Lys side chain lactam linkage to Glu
HXQGTFTSDYSKYLDEXAAKEFIQWLLQT

SEQ ID NO: 7 - protein, 29 aa, Artificial sequence
  Synthetic Peptide
  MOD_RES (2)..(2): Aib
  SITE (16)..(20): cyclic
  SITE (16)..(16): Glu side chain lactam linkage to Lys
  MOD_RES (17)..(17): Me17CO2H
  SITE (20)..(20): Lys side chain lactam linkage to Glu
HXQGTFTSDYSKYLDEKAAKEFIQWLLQT

SEQ ID NO: 8 - protein, 29 aa, Artificial sequence
  Synthetic Peptide
  MOD_RES (2)..(2): Aib
  SITE (16)..(20): cyclic
  SITE (16)..(16): Glu side chain lactam linkage to Lys
  SITE (20)..(20): Lys side chain lactam linkage to Glu
  MOD_RES (24)..(24): Lys(Z15CO2H)
HXQGTFTSDYSKYLDEQAAKEFIXWLLQT

SEQ ID NO: 9 - protein, 29 aa, Artificial sequence
  Synthetic Peptide
  MOD_RES (2)..(2): Aib
  SITE (16)..(20): cyclic
  SITE (16)..(16): Glu side chain lactam linkage to Lys
  SITE (20)..(20): Lys side chain lactam linkage to Glu
  MOD_RES (24)..(24): Lys(Z17CO2H)
HXQGTFTSDYSKYLDEQAAKEFIXWLLQT

SEQ ID NO: 10 - protein, 29 aa, Artificial sequence
  Synthetic Peptide
  MOD_RES (2)..(2): Aib
  MOD_RES (12)..(12): Arg
  SITE (16)..(20): cyclic
  SITE (16)..(16): Glu side chain lactam linkage to Lys
  MOD_RES (17)..(17): Lys(N-omega(1-(17-carboxyl-heptadecyloxy) beta-D-glucuronyl)) or Lys(Z17CO2H)
  SITE (20)..(20): Lys side chain lactam linkage to Glu
HXQGTFTSDYSXYLDEXAAKEFIQWLLQT

SEQ ID NO: 11 - protein, 31 aa, Artificial sequence
  Synthetic Peptide
  MOD_RES (2)..(2): Aib
  MOD_RES (20)..(20): Lys(yGlu-2xOEG)
HXEGTFTSDVSSYLEGQAAXEFIAWLVRGRG

SEQ ID NO: 12 - protein, 29 aa, Artificial sequence
  Synthetic Peptide
  MOD_RES (2)..(2): Aib
  SITE (16)..(20): cyclic
  SITE (16)..(16): Glu side chain lactam linkage to Lys
  SITE (16)..(20): Lys side chain lactam linkage to Glu
  MOD_RES (24)..(24): Lys(GC8)
HXQGTFTSDYSKYLDEQAAKEFIKWLLQT

SEQ ID NO: 13 - protein, 30 aa, Artificial sequence
  Synthetic Peptide
  misc_feature (2)..(2): Xaa can be any naturally occurring amino acid
  misc_feature (30)..(30): Xaa can be any naturally occurring amino acid
HXQGTFTSDYSKYLDEQAAKEFIKWLLQTX

SEQ ID NO: 14 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 15 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 16 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 17 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 18 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 19 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 20 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 21 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 22 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 23 - protein, 30 aa, Artificial sequence
  Synthetic Peptide
  misc_feature (2)..(2): Xaa can be any naturally occurring amino acid
  misc_feature (30)..(30): Xaa can be any naturally occurring amino acid
HXQGTFTSDYSKYLDEKAAKEFIQWLLQTX

SEQ ID NO: 24 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 25 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 26 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 27 - protein, 30 aa, Artificial sequence; identical to SEQ ID NO: 13
  Synthetic Peptide
  misc_feature (2)..(2): Xaa can be any naturally occurring amino acid
  misc_feature (30)..(30): Xaa can be any naturally occurring amino acid

SEQ ID NO: 28-29 - skipped (2 entries)

SEQ ID NO: 30 - protein, 31 aa, Artificial sequence
  Synthetic Peptide
HAEGTFTSDVSSYLEGQAAKEFIAWLVKGRG

SEQ ID NO: 31 - protein, 29 aa, Artificial sequence
  Synthetic Peptide
HSQGTFTSDYSKYLDSRRAQDFVQWLMNT

SEQ ID NO: 32 - protein, 30 aa, Artificial sequence
  Synthetic Peptide
  misc_feature (2)..(2): Xaa can be any naturally occurring amino acid
  misc_feature (30)..(30): Xaa can be any naturally occurring amino acid
HXQGTFTSDYSKYLDEQAAKEFICWLMNTX

SEQ ID NO: 33 - protein, 30 aa, Artificial sequence
  Synthetic Peptide
  misc_feature (2)..(2): Xaa can be any naturally occurring amino acid
  misc_feature (30)..(30): Xaa can be any naturally occurring amino acid
HXQGTFTSDYSKYLDEKAAKEFIKWKKQTX

(19) United States
(12) Patent Application Publication
(10) Pub. No.: US 2021/0290732 A1
(43) Pub. Date: Sep. 23, 2021

(54) GLP-1R and GCGR Agonists, Formulations, and Methods of Use

(71) Applicant: Spitfire Pharma LLC, Gaithersburg, MD (US)

(72) Inventors: John J Nestor, Sugar Land, TX (US); Vyjayanthi Krishnan, Gaithersburg, MD (US)

(73) Assignee: Spitfire Pharma LLC, Gaithersburg, MD (US)

(21) Appl. No.: 17/180,827

(22) Filed: Feb. 21, 2021

Related U.S. Application Data
(60) Provisional application No. 63/133,540, filed on Jan. 4, 2021.
(60) Provisional application No. 63/122,108, filed on Dec. 7, 2020.
(60) Provisional application No. 62/980,093, filed on Feb. 21, 2020.

(51) Int. Cl. A61K 38/26; A61K 47/10; A61P 3/04; A61K 9/00

(52) U.S. Cl. CPC A61K 38/26; A61K 9/0019; A61P 3/04; A61K 47/10

(57) ABSTRACT

This disclosure relates to the field of GLP-1R and GCGR agonists, formulations, and methods of using the same, including but not limited to dual agonist peptides of any of SEQ ID NOS. 1-10 or 12-27 conjugated to a non-ionic glycolipid surfactant.

### RELATED APPLICATIONS

This application claims priority to provisional application Nos. U.S. Ser. No. 62/980,093 filed 21 Feb. 2020; U.S. Ser. No. 63/122,108 filed 7 Dec. 2020; and, U.S. Ser. No. 63/133,540 filed 4 Jan. 2021 each of which are hereby incorporated into this application in their entirety.

### SEQUENCE LISTING

The instant application contains a Sequence Listing which has been submitted electronically in ASCII format via EFS-Web and hereby incorporated by reference in its entirety. Said ASCII copy, created on 27 May 2021, is named MED007US1_Corr2_ST25.TXT and is 24,576 bytes in size.

### FIELD OF THE DISCLOSURE

This disclosure relates to the field of GLP-1R and GCGR agonists, formulations, and methods of using the same.

### BACKGROUND OF THE DISCLOSURE

The increasing prevalence of obesity, diabetes mellitus, non-alcoholic fatty liver disease (NAFLD) and its advanced form, non-alcoholic steatohepatitis (NASH), is a world health crisis of epidemic proportions that is a major contributor to patient morbidity and mortality as well as a major economic burden. Obesity is an important risk factor for type 2 diabetes and NASH, and roughly 90% of patients with type 2 diabetes are overweight or obese. Obesity is a rapidly increasing problem worldwide and currently more than 65% of adults in the U.S. are overweight (Hedley, A. A., et al. (2004) JAMA 291: 2847-2850). NASH is anticipated to be the leading cause of liver transplant in the near future. There is a need for development of safe and efficacious pharmaceutical treatments for obesity and diabetes mellitus. The disclosure provides improved peptide pharmaceuticals for treatment of disorders associated with obesity or/and diabetes, such as non-alcoholic steatohepatitis (NASH) and polycystic ovary syndrome (PCOS).

In the United States (US), NASH has become the leading cause of end-stage liver disease or liver transplantation. Obesity is the core driver of NASH and weight loss results in reduction in liver fat and NASH improvement. More than 80% of individuals with NASH are overweight or obese, and with no currently available US Food and Drug Administration (FDA)-approved pharmacologic options for inducing weight loss, therapy has largely been based on lifestyle interventions directed at achieving weight loss. However, it is difficult to attain and maintain long-term weight loss with lifestyle changes alone.

Glucagon-like peptide-1 receptor agonists (GLP-1RA) are associated with modest degrees of weight loss at approved doses, and these agents have emerged as a treatment option for patients with NASH. In a recent clinical trial, liraglutide, a daily GLP-1RA, was associated with resolution of NASH, with a trend towards improvement of liver fibrosis. However, patients lost only 5.5% body weight. In one study, 10% or greater weight loss was required for optimal NASH resolution. Higher levels of weight loss have also been associated with lower incidences of cardiovascular disease and non-hepatic malignancies, which represent the most serious co-morbidities facing NASH patients.

GLP-1RAs exert central effects on appetite and food intake, while GCR agonists drive increased energy expenditure in animal models and humans. The effects of GCR agonist and GLP-1RA have been shown to be synergistic in driving greater degrees of weight loss compared to a GLP-1RA alone. GCRs also enhance lipolysis and suppress liver fat synthesis, providing an additional pathway for liver fat reduction and NASH resolution.

Dual agonists combine GCR with GLP-1RA in the same molecule. In obese non-human primates, chronic administration of a GLP-1R/GCR dual agonist reduced body weight and improved glucose tolerance to a greater degree compared to a GLP-1RA mono-agonist. Clinical studies of cotadutide, a GLP-1/GCR dual agonist with a 5:1 bias of GLP-1 to glucagon activity, demonstrated an impressive 39% reduction in liver fat content in just 6 weeks and greater improvement in NASH-related alanine aminotransferase (ALT) reduction than liraglutide alone. However, the degree of weight loss over 26 weeks of cotadutide administration was comparable to liraglutide (5.4% vs. 5.5%), suggesting that the 5:1 ratio was acceptable for liver fat reduction but suboptimal for weight reduction. Balanced (1:1) agonism has been shown to be associated with greater weight loss and metabolic effects than biased ratios that favor one agonist over the other. A recent study with JNJ 64565111, a balanced dual agonist, achieved an impressive 8% reduction in body weight in just 12 weeks (NCT03586830).

Unfortunately, GLP-1RAs have been associated with high rates of nausea, vomiting and diarrhea. These agents must also be titrated over prolonged periods to reduce side effects, and agents with improved tolerability and dosing regimens are needed. Accordingly, there remains a need for convenient dosing (e.g., weekly instead of daily) with a therapeutic dose to control blood glucose and/or induce weight loss that does not need to be titrated to reach a therapeutic level in the absence of gastrointestinal side effects.

### SUMMARY OF THE DISCLOSURE

Described herein are dual agonist peptides and products thereof (e.g., formulations) and uses of the same for treating disorders associated with the function of glucagon-like peptide 1 receptor (GLP-1R) and glucagon receptor (GCGR), including but not limited to insulin resistance or/and obesity, such as type 2 diabetes, metabolic syndrome, cardiovascular diseases (including coronary artery diseases such as atherosclerosis and myocardial infarction), hypertension, NASH, chronic kidney disease and PCOS, and in treating conditions associated with such disorders. Such dual agonist peptides have affinity for both GLP-1R and GCGR, as can be determined for example by a cellular assay as described herein or, using another assay for making such determinations. In some embodiments, the dual agonist peptide is any one of SEQ ID NOS. 1-10 or 12-27, or a derivative thereof, such as a conservatively substituted derivative thereof, and/or combinations thereof. In some embodiments, the dual agonist peptide exhibits about equal affinity for GLP-1R and GCGR as can be determined using the aforementioned cellular assay, which in preferred embodiments is SEQ ID NO: 1, or a derivative thereof.

In some embodiments, this disclosure provides pharmaceutical dosage formulation of such dual agonist peptide(s) configured to control blood glucose with reduction of one or more adverse events as compared to an agonist with unbalanced affinity for GLP-1R and GCGR (e.g., semaglutide) or with an excessively large maximal concentration in the blood following administration (Cmax). In some embodiments, this disclosure provides pharmaceutical dosage formulation of such dual agonist peptide(s) configured to induce weight loss with reduction of one or more adverse events as compared to an agonist with unbalanced affinity to GLP-1R and GCGR. The adverse events being in some embodiments selected from nausea, vomiting, diarrhea, abdominal pain and constipation, upon administration to a mammal. Those adverse events are typically observed following administration of a (dual) agonist with rapid entry into the circulation, leading to an excessively high Cmax. In contrast, the present pharmaceutical dosage formulation reduces or eliminates dosage-related adverse events, such as gastrointestinal (GI) adverse events, while providing a therapeutic dose for controlling blood glucose and/or treating obesity by inducing weight loss. In some embodiments, administration of the dual agonist peptide(s) disclosed herein (e.g., SEQ ID NOS. 1-10 or 12-27 or derivatives thereof) can result in improvements in other results (e.g., weight loss, fat loss, lipid profile) and/or pharmacokinetic (PK) parameters as compared to an agonist with unbalanced affinity for GLP-1R and GCGR (e.g., semaglutide). Other aspects of this disclosure are also contemplated as will be understood from the same by those of ordinary skill in the art.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1. Blood glucose response to subcutaneous (SC) injection of semaglutide or SEQ ID NO: 1 (db/db mice).

FIG. 2. Blood glucose response to semaglutide or SEQ ID NO: 1 (diet-induced obese (DIO) mice).

FIG. 3. Blood glucose IPGTT semaglutide or SEQ ID NO: 1 (DIO mice).

FIG. 4. Body weight response (% Day 0); SC injection of semaglutide or SEQ ID NO: 1 (db/db mice; leptin receptor-deficient mice).

FIG. 5. Feeding response to subcutaneous (SC) injection of semaglutide or SEQ ID NO: 1 (db/db mice).

FIGS. 6A and 6B. Body weight response (% Day 0) (FIG. 6A) and body weight response (g Day 0) (FIG. 6B). subcutaneous (SC) injection of semaglutide or SEQ ID NO: 1 (17) (DIO mice).

FIG. 7. Delta Fat Mass and Delta Lean Mass following administration of semaglutide or SEQ ID NO: 1.

FIG. 8. Ligand Concentrations of semaglutide and SEQ ID NO:1 measured over 120 hours, for a single dose administered subcutaneously (SC) to DIO mice.

FIG. 9. Ligand Concentrations of semaglutide and SEQ ID NO:1 (ALT-801) measured over 96 hours, for a single dose administered subcutaneously (SC) to C57BL/6J mice.

FIG. 10. Ligand Concentrations of semaglutide and SEQ ID NO:1 measured over 144 hours for a single dose, rats.

FIG. 11. Ligand Concentration of SEQ ID NO:1 measured over 360 hours, for a single dose administered intravenously (IV) or subcutaneously (SC) in Yucatan miniature swine.

FIG. 12A-D. Plasma ligand concentration (ng/mL) of SEQ ID NO: 1 measured over 192 hours (FIG. 12A) following three doses (10 nmol/kg (FIG. 12B), 20 nmol/kg (FIG. 12C), 40 nmol/kg (FIG. 12D)) administered subcutaneously (SC) in Cynomolgus monkeys.

FIG. 13. Body weight change in male cynomolgus treated with SEQ ID NO: 1 (0.03 mg/kg to 0.25 mg/kg).

FIG. 14. Body weight change in female cynomolgus treated with SEQ ID NO: 1 (0.03 mg/kg to 0.25 mg/kg).

FIG. 15. Body weight of treatment groups (NASH mice) with SEQ ID NO: 1 (ALT-801) as compared to semaglutide and elafibranor.

FIG. 16. Change in NAFLD Activity Score under treatment with SEQ ID NO: 1 (ALT-801) as compared to semaglutide and elafibranor.

FIG. 17. Treatment improved liver morphology, liver weight, NAS, and fibrosis with SEQ ID NO: 1 (ALT-801) as compared to semaglutide and elafibranor.

FIG. 18. Mean terminal liver TG, liver TC, and plasma ALT with SEQ ID NO: 1 (ALT-801) as compared to semaglutide and elafibranor.

FIG. 19. Modulation of Gene Expression by ALT-801 (SEQ ID NO: 1).

FIG. 20. Modulation of genes affecting fat usage and transport following treatment with SEQ ID NO: 1 (ALT-801) and semaglutide.

FIG. 21. Modulation of liver stellate cell pathway pro-fibrosis, cell death, and inflammation genes following treatment with SEQ ID NO: 1 (ALT-801) and semaglutide.

FIG. 22. In vitro stability in human plasma. See Table 14.

FIG. 23. In vivo pharmacokinetic behavior of compounds following sc administration to Gottingen mini pigs.

FIG. 24. In vivo PK behavior of SEQ ID NO: 1 and semaglutide following subcutaneous (sc) administration.

FIG. 25. In vivo pharmacokinetic behavior of SEQ ID NO: 1 following single subcutaneous (sc) and intravenous (iv) administration to male mini-swine (n=4; wt circa 75 kg) at 20 nmol/kg.

FIG. 26. In vivo dose response behavior of 17 (SEQ ID NO: 1) and literature standard semaglutide following subcutaneous (sc) administration of single dose, in male db/db mice (n=9).

FIG. 27. Body weight of male DIO rats (n=9) during 28 day treatment (followed by recovery) with vehicle, literature standard semaglutide (12 nmol/kg), SEQ ID NO: 1 (6 and 12 nmol/kg), and groups pair-fed to the amount of food consumed by the animals in the 12 nmol/kg semaglutide and SEQ ID NO: 1 groups.

FIG. 28. Cumulative food consumption by DIO rats during 27 day treatment (followed by recovery) with vehicle, literature standard semaglutide (12 nmol/kg), SEQ ID NO: 1 (6 and 12 nmol/kg), and groups pair-fed to the amount of food consumed by the animals in the 12 nmol/kg semaglutide or SEQ ID NO: 1 groups.

FIG. 29. Daily food consumption by DIO rats during 27 day treatment in response to daily subcutaneous (sc) doses of with vehicle, literature standard semaglutide (12 nmol/kg), SEQ ID NO: 1 (6 and 12 nmol/kg), and groups pair-fed to the amount of food consumed by the animals treated with daily sc 12 nmol/kg semaglutide or SEQ ID NO: 1 groups.

FIG. 30. Surface tension data for ALT-801 in pure water.

### DETAILED DESCRIPTION OF THE DISCLOSURE

This disclosure relates to a dual agonist peptide(s) as well as pharmaceutical dosage formulations comprising, and methods for using, the same. The dual agonist peptides have affinity for, and in preferred embodiments about equal affinity for, glucagon-like peptide 1 receptor (GLP-1R) and glucagon receptor (GCGR), as may be determined using a cellular assay. In some embodiments, this disclosure provides pharmaceutical dosage formulations configured to control blood glucose. In some embodiments, blood glucose is better controlled (e.g., lowered and stabilized) following administration of a dual agonist peptide as compared to a selective (e.g., semaglutide) and/or unbalanced agonist. In some embodiments, this disclosure provides pharmaceutical dosage formulations configured to induce weight loss. In some embodiments, weight loss is improved (e.g., lowered and/or stabilized) following administration of a dual agonist peptide as compared to a selective (e.g., semaglutide) and/or unbalanced agonist. In some embodiments, such pharmaceutical dosage formulations exhibit a reduction in adverse events as compared to an agonist with selective (e.g., semaglutide) and/or unbalanced affinity for GLP-1R and GCGR. In some embodiments, the adverse events can include nausea, vomiting, diarrhea, abdominal pain and/or constipation, that are typically observed following administration of upon administration an agonist with unbalanced affinity for GLP-1R and GCGR (e.g., semaglutide) to a mammal. In some embodiments, this disclosure provides novel peptide-based dual GLP-1/glucagon receptor agonists designed to treat the underlying metabolic dysfunction that leads to non-alcoholic steatohepatitis (NASH).

In some embodiments, the dual agonist peptide is any one of SEQ ID NOS: 1-10 or 12-27, or a derivative thereof.

In preferred embodiments, the dual agonist peptide is EU-A1873 (SEQ ID NO: 1), EU-A1588 (SEQ ID NO: 2), EU-A1871 (SEQ ID NO: 3), EU-A1872 (SEQ ID NO: 4), as shown in Table 1:

TABLE 1

| | SEQ. | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|
| | ID. | | | | | | | | | | | | | | | | | | | | | | | | | | | | | | | |
| | NO. | 1 | | | | 5 | | | | | 10 | | | | | 15 | | | | | 20 | | | | | 25 | | | | | 30 | |
| sema- | 11 | H | Aib | E | G | T | F | T | S | D | V | S | S | Y | L | E | G | Q | A | A | Lys | E | F | I | A | W | L | V | R | G | R | G |
| glutide | | | | | | | | | | | | | | | | | | | | | (EPPC17CO2H) | | | | | | | | | | | |
| EU-A1873 | 1 | H | Aib | Q | G | T | F | T | S | D | Y | S | S | Y | L | D | E* | Lys | A | A | K* | E | F | I | Q | W | L | | Q | T | NH₂ | |
| | | | | | | | | | | | | | | | | | | (Z17CO2H) | | | | | | | | | | | | | | |
| EU-A1588 | 2 | H | Aib | Q | G | T | F | T | S | D | Y | S | S | Y | L | D | E* | | A | A | K* | E | F | I | Q | W | L | | Q | T | NH₂ | |
| EU-A1871 | 3 | H | Aib | Q | G | T | F | T | S | D | Y | S | S | Y | L | D | E* | | A | A | K* | E | F | I | Lys(Z15CO2H) | W | L | | Q | T | NH₂ | |
| EU-A1872 | 4 | H | Aib | Q | G | T | F | T | S | D | Y | S | S | Y | L | D | E* | | A | A | K* | E | F | I | Lys(Z17CO2H) | W | L | | Q | T | NH₂ | |
| EU-A1880 | 5 | H | Aib | Q | G | T | F | T | S | D | Y | S | S | Y | L | D | E* | | A | A | K* | E | F | I | Q | W | L | | Q | T | NH₂ | |

E* and K* indicate a side chain lactam linkage between these residues
(EPC17CO2H = (17-carboxyhepadecanoyl-(γ-Glu)-AEEA-REEA); Z17CO2H = (beta-D-glucuron-1-yl)-1-oxa)17-carboxyheptadecane:
Me15CO2H = (beta-D-melobiouranyl-1-yl)-1-oxa)15-carboxypentadecane

In Table 1, the numbers 1, 5, 10, 15, 20, 25 and 30 in the top row refer to amino acid residue numbers (29 total amino acid residues being present in each of SEQ ID NOS: 1-5). Semaglutide shown in Table 1 is SEQ ID NO: 11 (31 amino acid residues). As shown in Table 1, SEQ ID NO: 1 (EU-A1873 of Table 1; wherein ALT-801 is the active pharmaceutical ingredient (API) present in the disclosed pharmaceutical formulation, wherein the API is represented by SEQ ID NO: 1) has the following amino acid sequence conjugated at amino acid position 17 (aa17) to the non-ionic glycolipid surfactant:

¹His-²Aib-³Gln-⁴Gly-⁵Thr-⁶Phe-⁷Thr-⁸Ser-⁹Asp-¹⁰Tyr-
¹¹Ser-¹²Lys-¹³Tyr-¹⁴Leu-¹⁵Asp-¹⁶Glu*-¹⁷Lys^{#}-¹⁸Ala-¹⁹Ala-
²⁰Lys*-²¹Glu-²²Phe-²³Ile-²⁴G1n-²⁵Trp-²⁶Leu-²⁷Leu-²⁸Gln-
²⁹Thr-NH₂,

where * indicates a lactam bridge is formed between Glu16 and Lys 20, and 17Lys^{#} indicates the attachment site for glucuronic acid C-18 (EuPort, Z17CO₂H also referred to herein as GC18c).
   Illustrated differently, SEQ ID NO: 1 is a peptide amide consisting of 29 amino acid residues and a glucuronic acid/C18 diacid moiety attached to ¹⁷Lys, in which the side-chains of ¹⁶Glu and ²⁰Lys forming an intramolecular cycle as shown below:

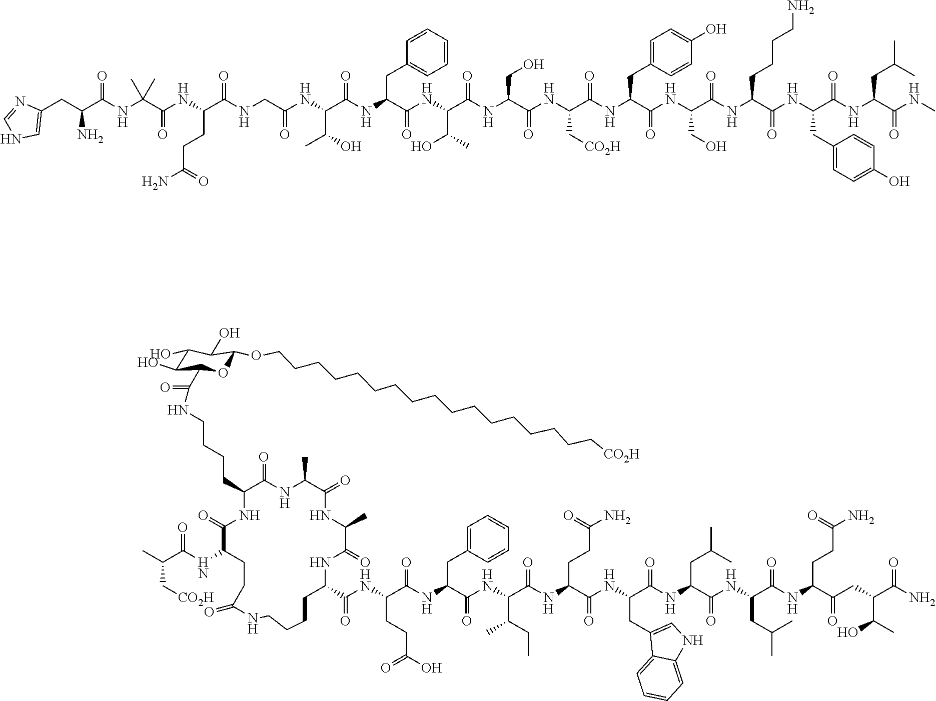

In some embodiments, the dual agonist peptide can be any of:

```
(SEQ ID NO: 6)
His Xaa1 Gln Gly Thr Phe Thr Ser Asp Tyr Ser Lys
      1 5 10
Tyr Leu Asp Glu Xaa2 Ala Ala Lys Glu Phe Ile Gln
                      15 20
Trp Leu Leu Gln Thr
25
```

wherein: Xaa1 is any amino acid, preferably Aib (α-aminoisobutyric acid (or 2-methylalanine or Calpha-methylalanine)); Xaa2 is Lys(N-omega(1-(17-carboxyl-heptadecyloxy)beta-D-glucuronyl)) or Lys(Z17CO2H) where Z17CO2H (EuPort) is (beta-D-glucuron-1-yl)-1-oxa)17-carboxyheptadecane; and, Glu16 and Lys20 are cyclized with one another through their respective side chains to form a lactam linkage; or a derivative thereof;

```
(SEQ ID NO: 7)
His Xaa1 Gln Gly Thr Phe Thr Ser Asp Tyr Ser Lys
      1 5 10
Tyr Leu Asp Glu Xaa2 Ala Ala Lys Glu Phe Ile Gln
                      15 20
Trp Leu Leu Gln Thr
      25
```

wherein: Xaa1 is any amino acid, preferably Aib (α-aminoisobutyric acid (or 2-methylalanine or Calpha-methylalanine)); Xaa2 is Lys(Me17CO2H), where Me17CO2H is (beta-D-melobiouranyl-1-yl)-1-oxa)17-carboxyheptadecane; and, Glu16 and Lys20 are cyclized with one another through their respective side chains to form a lactam linkage; or a derivative thereof;

```
(SEQ ID NO: 8)
His Xaa1 Gln Gly Thr Phe Thr Ser Asp Tyr Ser Lys
      1 5 10
Tyr Leu Asp Glu Gln Ala Ala Lys Glu Phe Ile Xaa3
                      15 20
Trp Leu Leu Gln Thr
        25
```

wherein: Xaa1 is any amino acid, preferably Aib (α-aminoisobutyric acid (or 2-methylalanine or Calpha-methylalanine); Glu16 and Lys20 are cyclized with one another through their respective side chains to form a lactam linkage; Xaa3 is Lys(Z15CO2H) where Z15CO2H is (beta-D-glucuron-1-yl)-1-oxa)15-carboxyheptadecane; or a derivative thereof;

```
(SEQ ID NO: 9)
His Xaa1 Gln Gly Thr Phe Thr Ser Asp Tyr Ser Lys
      1 5 10
Tyr Leu Asp Glu Gln Ala Ala Lys Glu Phe Ile Xaa4
                      15 20
Trp Leu Leu Gln Thr
        25
```

wherein: Xaa1 is any amino acid, preferably Aib (α-aminoisobutyric acid (or 2-methylalanine or Calpha-methylalanine); Glu16 and Lys20 are cyclized with one another through their respective side chains to form a lactam linkage; Xaa4 is Lys(Z17CO2H) where Z17CO2H is (beta-D-glucuron-1-yl)-1-oxa)17-carboxyheptadecane; or a derivative thereof;
or,

```
(SEQ ID NO: 10)
His Xaa1 Gln Gly Thr Phe Thr Ser Asp Tyr Ser Xaa5
      1 5 10
Tyr Leu Asp Glu Xaa2 Ala Ala Lys Glu Phe Ile Gln
                15 20
Trp Leu Leu Gln Thr
      25
```

wherein: Xaa1 is any amino acid, preferably Aib (α-aminoisobutyric acid (or 2-methylalanine or Calpha-methylalanine)); Xaa2 is Lys(N-omega(1-(17-carboxyl-heptadecyloxy)beta-D-glucuronyl)) or Lys(Z17CO2H) where Z17CO2H is (beta-D-glucuron-1-yl)-1-oxa)17-carboxyheptadecane; Xaa5 is Arg, and, Glu16 and Lys20 are cyclized with one another through their respective side chains to form a lactam linkage; or a derivative thereof.

In some embodiments, the dual agonist peptide is selected from the group consisting of SEQ ID Nos. 1 and 12-27 shown below:

| | | | | | | | | | | | | | | | | | | | | | | | | | | | | | | | | SEQ ID |
|---|---|---|---|---|---|---|---|---|---|---|---|---|---|---|---|---|---|---|---|---|---|---|---|---|---|---|---|---|---|---|---|---|
| cmpd # | 1 | | | | 5 | | | | | 10 | | | | | 15 | | | | | 20 | | | | | 25 | | | | | 30 | | NO. |
| Ref 27, sema | H | Aib | E | G | T | F | T | S | D | V | S | S | Y | L | E | G | Q | A | A | X | E | F | I | A | W | L | V | R | G | R | G | 11 |
| GLP-17-37 | H | A | E | G | T | F | T | S | D | V | S | S | Y | L | E | G | Q | A | A | X | E | F | I | A | W | L | V | K | G | R | G | 30 |
| Glucagon | H | S | Q | G | T | F | T | S | D | Y | S | K | Y | L | D | S | R | R | A | Q | D | F | V | Q | W | L | M | N | T | | | 31 |
| Ref 8, #32 | H | Aib | Q | G | T | F | T | S | D | Y | S | K | Y | L | D | E* | Q | A | A | K* | E | F | I | C | W | L | M | N | T | NH₂ | | 32 |
| 1 | H | Aib | Q | G | T | F | T | S | D | Y | S | K | Y | L | D | E* | Q | A | A | K* | E | F | I | Lys(GC8) | W | L | L | Q | T | NH₂ | | 12 |
| 2 | H | Aib | Q | G | T | F | T | S | D | Y | S | K | Y | L | D | E* | Q | A | A | K* | E | F | I | Lys(GC10) | W | L | L | Q | T | NH₂ | | 13 |
| 3 | H | Aib | Q | G | T | F | T | S | D | Y | S | K | Y | L | D | E* | Q | A | A | K* | E | F | I | Lys(GC12) | W | L | L | Q | T | NH₂ | | 14 |
| 4 | H | Aib | Q | G | T | F | T | S | D | Y | S | K | Y | L | D | E* | Q | A | A | K* | E | F | I | Lys(GC14) | W | L | L | Q | T | NH₂ | | 15 |
| 5 | H | Aib | Q | G | T | F | T | S | D | Y | S | K | Y | L | D | E* | Q | A | A | K* | E | F | I | Lys(GC16) | W | L | L | Q | T | NH₂ | | 16 |
| 6 | H | Aib | Q | G | T | F | T | S | D | Y | S | K | Y | L | D | E* | Q | A | A | K* | E | F | I | Lys(GC18) | W | L | L | Q | T | NH₂ | | 17 |
| 7 | H | Aib | Q | G | T | F | T | S | D | Y | S | K | Y | L | D | E* | Q | A | A | K* | E | F | I | Lys(MC12) | W | L | L | Q | T | NH₂ | | 18 |
| 8 | H | Aib | Q | G | T | F | T | S | D | Y | S | K | Y | L | D | E* | Q | A | A | K* | E | F | I | Lys(MeC12) | W | L | L | Q | T | NH₂ | | 19 |
| 9 | H | Aib | Q | G | T | F | T | S | D | Y | S | K | Y | L | D | E* | Q | A | A | K* | E | F | I | Lys(MeC14) | W | L | L | Q | T | NH₂ | | 20 |
| 10 | H | Aib | Q | G | T | F | T | S | D | Y | S | K | Y | L | D | E* | Q | A | A | K* | E | F | I | Lys(MeC16) | W | L | L | Q | T | NH₂ | | 21 |
| 11 | H | Aib | Q | G | T | F | T | S | D | Y | S | K | Y | L | D | E* | Q | A | A | K* | E | F | I | Lys(MeC18) | W | L | L | Q | T | NH₂ | | 22 |
| 12 | H | Aib | Q | G | T | F | T | S | D | Y | S | K | Y | L | D | E* | Lys(MeC14) | A | A | K* | E | F | I | Q | W | L | L | Q | T | NH₂ | | 23 |
| 13 | H | Aib | Q | G | T | F | T | S | D | Y | S | K | Y | L | D | E* | Q | A | A | K* | E | F | I | Lys | W | L | L | Q | T | NH₂ | | 24 |
| | | | | | | | | | | | | | | | | | | | | | | | | (S₁GC14) | | | | | | | | |
| 14 | H | Aib | Q | G | T | F | T | S | D | Y | S | K | Y | L | D | E* | Q | A | A | K* | E | F | I | Lys | W | L | L | Q | T | NH₂ | | 25 |
| | | | | | | | | | | | | | | | | | | | | | | | | (S₂GC14) | | | | | | | | |
| 15 | H | Aib | Q | G | T | F | T | S | D | Y | S | K | Y | L | D | E* | Q | A | A | K* | E | F | I | Lys(GC16c) | W | L | L | Q | T | NH₂ | | 26 |
| 16 | H | Aib | Q | G | T | F | T | S | D | Y | S | K | Y | L | D | E* | Q | A | A | K* | E | F | I | Lys(GC18c) | W | L | L | Q | T | NH₂ | | 27 |
| 17 | H | Aib | Q | G | T | F | T | S | D | Y | S | K | Y | L | D | E* | Lys(GC18c) | A | A | K* | E | F | I | Q | W | L | L | Q | T | NH₂ | | 1 |

Starred analogs have a Glu15 to Lys20 side chain lactam; G, M, Me in parentheses means D-glucoside, D-maltoside, D-melibioside linkages, respectively.
S₁ and S₂ mean a spacer of α-Lys or γ-Glu residue, respectively.
Cn means methylene chain of n carbons; c means carboxylate at end of chain.
X in semaglutide means a Lys residue acylated with γGlu-2xOEG (see ref 27) prolongation modifier comprising octadecandioic acid on a γGlu/short-PEG spacer.
Cmpd #33 in reference 8 refers to Cmpd #32 alkylated on Cys 24 with a 40 kDa PEG through a maleimide linker.

In preferred embodiments, the dual agonist peptide is one having the amino acid sequence of any one of SEQ ID NOS: 1-10 or 12-27, or a derivative thereof. In preferred embodiments, the dual agonist peptide is SEQ ID NO: 1. In some embodiments, the dual agonist peptide is formulated as a solution for injection comprising pharmaceutically acceptable excipients such as a osmolarity adjusting agent or salt, a buffering agent, an stabilizing agent and/or a surfactant, a pH adjuster and a solvent. In some embodiment, the osmolarity adjusting agent is mannitol, sorbitol, glycerol, and glycine, propylene glycol or sodium chloride. In some embodiments, the buffering agent is histidine arginine, lysine, phosphate, acetate, carbonate, bicarbonate, citrate, Meglumine or Tris. In some embodiments, the stabilizing agent is histidine, arginine or lysine. In some embodiments, the surfactant is polysorbate 20 or polysorbate 80. In some embodiment, the pH adjuster is hydrochloric acid and/or sodium hydroxide. In preferred embodiment, the osmolarity adjusting agent is mannitol, the buffering agent and stabilizing agent is arginine, and the surfactant is a polysorbate 20. In some embodiments, the dual agonist peptide can be formulated as a pharmaceutical dosage formulation comprising about 0.025-0.15% (w/w) polysorbate 20, about 0.2-0.5% (w/w) arginine, and about 3-6% (w/w) mannitol in deionized water (pH 7.7±1.0). In some embodiments, the pharmaceutical dosage formulation comprises “ALT-801” represented by SEQ ID NO: 1 in a formulation comprising, consisting essentially of, or consisting of, about 0.050% (w/w) polysorbate 20, about 0.35% (w/w) arginine, and about 4.3% (w/w) mannitol in deionized water (pH 7.7±1). As used herein, the test article formulation is also referred to as F58 formulation. See Example 4. In preferred embodiments, the pharmaceutical dosage formulation for “ALT-801” comprises SEQ ID NO: 1 in a formulation comprising, consisting essentially of, or consisting of, about 0.35% (w/w) arginine, and about 4.3% (w/w) mannitol 0.6 to 1.0 mg of polysorbate 20 per mg of “ALT-801” (SEQ ID NO:1) or 1.0 to 1.5 mg of polysorbate 80 per mg of “ALT-801” (SEQ ID NO:1). See Example 8. In some embodiment, the pharmaceutical dosage formulation comprises “ALT-801” at a concentration ranging from 0.05 mg/ml to 20 mg/ml, preferably from 0.1 mg/ml to 10 mg/ml or more preferably 0.5 mg/mg to 10 mg/ml. In some embodiments, the pH of the pharmaceutical dosage formulation comprising “ALT-801” is from 6 to 10, more preferably 6 to 8.

The synthesis of the dual agonist peptides including the non-ionic glycolipid surfactant (e.g., SEQ ID NOS: 1-10 or 12-27, or derivatives thereof) is described herein (e.g., Example 1) and in U.S. Pat. No. 9,856,306 B2, which is incorporated by reference in its entirety into this disclosure. In some embodiments, the dual agonist peptides can include one or more conservatively substituted amino acids as described herein. In preferred embodiments, SEQ ID NO: 1 can include one or more conservatively substituted amino acids, but preferably not at amino acid residues 16, 17, or 20. In preferred embodiments, SEQ ID NO: 2 can include one or more conservatively substituted amino acids, but preferably not at amino acid residues 16, 17, or 20. In preferred embodiments, SEQ ID NO: 3 can include one or more conservatively substituted amino acids, but preferably not at amino acid residues 16, 20, or 24. In preferred embodiments, SEQ ID NO: 4 can include one or more conservatively substituted amino acids, but preferably not amino acid residues 16, 20, or 24, SEQ ID NO:5 can include one or more conservatively substituted amino acids, but preferably not amino acid residues 12, 16, 17, or 20.

The peptides of SEQ ID NOS: 1-10 or 12-27 can be collectively referred to herein as the “dual agonist peptides” (or individually as “dual agonist peptide”) as each is an agonist for the glucagon-like peptide 1 receptor (GLP-1R) and glucagon receptor (GCGR). In some embodiments, the peptide is a dual agonist of GLP-1R and GCGR as can be determined by a cellular assay such as that described in Example 2 herein. Briefly, in some embodiments, cellular assays can be carried out by measuring cAMP stimulation or arrestin activation in CHO cells into which human GLP-IR or GCGR are expressed ((LeadHunter assays (DiscoveRx)). Preferably, such assays are carried out in the presence of 0.1% ovalbumin as compared to 0.1% bovine serum albumin (BSA) as may be typical, since the dual agonist peptides of SEQ ID NOS: 1-10 or 12-27 can bind very tightly to serum albumin (>99%) and distort the results (see, e.g., Example 2 herein). In some embodiments, as determined using such assays, the dual agonist peptide can have affinity for both GLP-1R and GCGR, and in preferred embodiments about equal affinity for GLP-1R and GCGR. “About equal affinity” means that the dual agonist peptide has no more than about two to three times, preferably not more than two times, the affinity for GLP-1R or GCGR as for the other, as can be determined by such a cellular assay. For instance, as shown in the Examples herein, the dual agonist peptide SEQ ID NO: 1 (EU-A1873) has been surprisingly found to be a dual agonist peptide with about equal affinity for GLP-1R and GCGR (e.g., an EC50 of about 39 pm (115% intrinsic activity) for GLP-1R and 44 pm (115% intrinsic activity) for GCGR). This is unlike the GLP-1 “specific” compounds including semaglutide and Exendin-4, that present affinity strongly biased toward, or only for, GLP-1R; or the strongly GCGR-biased hormone glucagon, which do not show high, or about equal, affinity for both of GLP-1R and GCGR. The native hormone oxyntomodulin has agonistic action at both GLP-1 and glucagon receptors, but this activity is not potent and is not balanced. Those of ordinary skill in the art will understand that affinity to GLP-1R and GCGR can be determined by methods and/or assays other than those described herein and that such methods and/or assays for determining affinity are contemplated herein (e.g., a determination of about equal affinity can be made by such other methods and/or assays).

In embodiments, “a dual agonist peptide with about equal affinity for glucagon-like peptide 1 receptor (GLP-1R) and glucagon receptor (GCGR)” as used herein means a dual agonist peptide that has no more than about two times the affinity for GLP-1R or GCGR as for the other, as can be determined by such a cellular assay. In embodiments, the binding affinity of the present dual agonist peptide for one receptor as compared to the other is no more than 1.9, 1.8, 1.6, 1.5, 1.4, or 1.2 times, as can be determined by known cellular assays. In embodiments, “an agonist with unbalanced affinity for GLP-1R and GCGR” as used herein means an agonist peptide that has at least about 1.5 times the affinity for GLP-1R or GCGR as for the other, as can be determined by known cellular assays. In embodiments, the binding affinity of an agonist with an unbalanced affinity for GLP-1R and GCGR is at least 1.6, 1.8, 2, 2.5, 3, 5, 7.5, 10, 20 times, or more as can be determined by known cellular assays.

A “peptide” (e.g., dual agonist peptide) comprises two or more natural or/and unnatural amino acid residues linked typically via peptide bonds. Such amino acids can include naturally occurring structural variants, naturally occurring non-proteinogenic amino acids, or/and synthetic non-naturally occurring analogs of natural amino acids. The terms “peptide” and “polypeptide” are used interchangeably herein. Peptides include short peptides (about 2-20 amino acids), medium-length peptides (about 21-50 amino acids) and long peptides (>about 50 amino acids, which can also be called “proteins”). In some embodiments, a peptide product comprises a surfactant moiety covalently and stably attached to a peptide of no more than about 50, 40 or 30 amino acids. Synthetic peptides can be synthesized using an automated peptide synthesizer, for example. Peptides can also be produced recombinantly in cells expressing nucleic acid sequences that encode the peptides. Conventional notation is used herein to portray peptide sequences: the left-hand end of a peptide sequence is the amino (N)-terminus, and the right-hand end of a peptide sequence is the carboxyl (C)-terminus. Standard one-letter and three-letter abbreviations for the common amino acids are used herein. Although the abbreviations used in the amino acid sequences disclosed herein represent L-amino acids unless otherwise designated as D- or DL- or the amino acid is achiral, the counterpart D-isomer generally can be used at any position (e.g., to resist proteolytic degradation). Abbreviations for other amino acids used herein include: Aib=a-aminoisobutyric acid (or 2-methylalanine or Ca-methylalanine); Xaa: any amino acid, typically specifically defined within a formula. Abbreviations for other amino acids that can be used as described herein include: Ac3c=1-aminocyclopropane-1-carboxylic acid; Ac4c=1-aminocyclobutane-1-carboxylic acid; Ac5c=1-aminocyclopentane-1-carboxylic acid; Ac6c=1-aminocyclohexane-1-carboxylic acid; Aib=alpha-aminoisobutyric acid (or 2-methylalanine or Calpha-methylalanine); Bip=3-(biphenyl-4-yl)alanine; Bip2Et=3-(2′-ethylbiphenyl-4-yl)alanine; Bip2EtMeO=3-(2′-ethyl-4′-methoxybiphenyl-4-yl)alanine; Cit=citrulline; Deg=2,2-diethylglycine; Dmt=(2,6-dimethyl)tyrosine; 2FPhe=(2-fluorophenyl)alanine; 2FMePhe or 2FaMePhe=Ca-methyl-(2-fluorophenyl)alanine; hArg=homoarginine; MeLys or aMeLys=Ca-methyllysine; MePhe or aMePhe=Ca-methylphenylalanine; MePro or aMePro=Ca-methylproline; Nal1 or Nal(1)=3-(1-naphthyl)alanine; Nal2 or Nal(2)=3-(2-naphthyl)alanine; Nle=norleucine; Om=ornithine; and Tmp=(2,4,6-trimethylphenyl)alanine; 1,2,3,4-tetrahydroisoquinoline-3-carboxylic acid (Tic) and a Tic-Phe dipeptide moiety with a reduced amide bond between the residues (designated as Tic-Ψ[CF12-NF1]-Ψ-Phe) have the following structures:

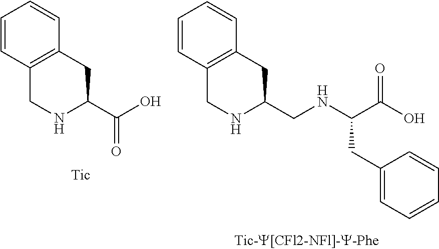 [SMILES: O=C(O)[C@@H]1Cc2ccccc2CN1 ; O=C(O)[C@H](Cc1ccccc1)NC[C@@H]1Cc2ccccc2CN1]

Unless specifically stated otherwise or the context clearly indicates otherwise, the disclosure encompasses any and all forms of a dual agonist peptide that may be produced, whether the dual agonist peptide is produced synthetically (e.g., using a peptide synthesizer) or by a cell (e.g., by recombinant production). Such forms of a dual agonist peptide can include one or more modifications that may be made during the course of synthetic or cellular production of the peptide, such as one or more post-translational modifications, whether or not the one or more modifications are deliberate. A dual agonist peptide can have the same type of modification at two or more different places, or/and can have two or more different types of modifications. Modifications that may be made during the course of synthetic or cellular production of a dual agonist peptide, including chemical and post-translational modifications, include without limitation glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination. A dual agonist peptide can have one or more modifications anywhere, such as the N-terminus, the C-terminus, one or more amino acid side chains, or the dual agonist peptide backbone, or any combination thereof. In some embodiments, a dual agonist peptide is acetylated at the N-terminus or/and has a carboxamide (—CONH₂) group at the C-terminus, which can increase the stability of the dual agonist peptide.

Potential modifications of a dual agonist peptide also include deletion of one or more amino acids, addition/insertion of one or more natural or/and unnatural amino acids, or substitution with one or more natural or/and unnatural amino acids, or any combination or all thereof. A substitution can be conservative or non-conservative. Such modifications may be deliberate, such as via site-directed mutagenesis or in the chemical synthesis of a dual agonist peptide, or may be accidental, such as via mutations arising in the host cell that produces the dual agonist peptide or via errors due to PCR amplification. An unnatural amino acid can have the same chemical structure as the counterpart natural amino acid but have the D stereochemistry, or it can have a different chemical structure and the D or L stereochemistry. Unnatural amino acids can be utilized, e.g., to promote a-helix formation or/and to increase the stability of the dual agonist peptide (e.g., to resist proteolytic degradation). A dual agonist peptide having one or more modifications relative to a reference dual agonist peptide may be called an “analog” or “variant” of the reference dual agonist peptide as appropriate. An “analog” typically retains one or more essential properties (e.g., receptor binding, activation of a receptor or enzyme, inhibition of a receptor or enzyme, or other biological activity) of the reference dual agonist peptide. A “variant” may or may not retain the biological activity of the reference dual agonist peptide, or/and may have a different biological activity. It is preferred that such a variant maintain its ability to act as an agonist of GLP-1R and GCGR, and in more preferred embodiments, has about equal affinity for GLP-1R and GCGR. In some embodiments, an analog or variant of a reference peptide has a different amino acid sequence than the reference dual agonist peptide.

The term “conservative substitution” refers to substitution of an amino acid in a dual agonist peptide with a functionally, structurally or chemically similar natural or unnatural amino acid. In certain embodiments, the following groups each contain natural amino acids that are conservative substitutions for one another: 1) Glycine (Gly/G), Alanine (Ala/A); 2) Isoleucine (Ile/I), Leucine (Leu/L), Methionine (Met/M), Valine (Val/V); 3) Phenylalanine (Phe/F), Tyrosine (Tyr/Y), Tryptophan (Trp/W); 4) Serine (Ser/S), Threonine (Thr/T), Cysteine (Cys/C); 5) Asparagine (Asn/N), Glutamine (Gln/Q); 6) Aspartic acid (Asp/D), Glutamic acid (Glu/E); and, 7) Arginine (Arg/R), Lysine (Lys/K), Histidine (His/H). In further embodiments, the following groups each contain natural amino acids that are conservative substitutions for one another: 1) non-polar: Ala, Val, Leu, Ile, Met, Pro (proline/P), Phe, Trp; 2) hydrophobic: Val, Leu, Ile, Phe, Trp; 3) aliphatic: Ala, Val, Leu, Ile; 4) aromatic: Phe, Tyr, Trp, His; 5) uncharged polar or hydrophilic: Gly, Ala, Pro, Ser, Thr, Cys, Asn, Gln, Tyr; 6) aliphatic hydroxyl- or sulfhydryl-containing: Ser, Thr, Cys; 7) amide-containing: Asn, Gln; 8) acidic: Asp, Glu; 9) basic: Lys, Arg, His; and, 10) small: Gly, Ala, Ser, Cys. In other embodiments, amino acids may be grouped as conservative substitutions as set out below: 1) hydrophobic: Val, Leu, Ile, Met, Phe, Trp; 2) aromatic: Phe, Tyr, Trp, His; 3) neutral hydrophilic: Gly, Ala, Pro, Ser, Thr, Cys, Asn, Gln; 4) acidic: Asp, Glu; 5) basic: Lys, Arg, His; and, 6) residues that influence backbone orientation: Pro.

Examples of unnatural or non-proteinogenic amino acids include without limitation alanine analogs (e.g., α-ethylGly [α-aminobutyric acid or Abu], α-n-propylGly [norvaline or Nva], α-tert-butylGly [Tbg], α-vinyl Gly [Vg or Vlg], α-allylGly [Alg], α-propargylGly [Prg], 3-cyclopropylAla [Cpa] and Aib), leucine analogs (e.g., nor-leucine, Nle), proline analogs (e.g., α-MePro), phenylalanine analogs (e.g., Phe(2-F), Phe(2-Me), Tmp, Bip, Bip(2′-Et-4′-OMe), Nal1, Nal2, Tic, α-MePhe, α-MePhe(2-F) and α-MePhe(2-Me)), tyrosine analogs (e.g., Dmt and α-MeTyr), serine analogs (e.g., homoserine [isothreonine or hSer]), glutamine analogs (e.g., Cit), arginine analogs (e.g., hArg, N,N′-g-dialkyl-hArg), lysine analogs (e.g., homolysine [hLys], Orn and α-MeLys), α, α-disubstituted amino acids (e.g., Aib, α, α-diethylGly [Deg], α-cyclohexylAla [2-Cha], Ac3c, Ac4c, Ac5c and Ac6c), and other unnatural amino acids disclosed in A. Santoprete et al., Pept. Sci., 17:270-280 (2011). α,α-Di-substituted amino acids can provide conformational restraint or/and a-helix stabilization. A reduced amide bond between two residues (as in, e.g., Tic-Ψ[CF12-NF1]-Ψ-Phe) increases protease resistance and may also, e.g., alter receptor binding. The disclosure encompasses all pharmaceutically acceptable salts of dual agonist peptides, including those with a positive net charge, those with a negative net charge, and those with no net charge.

An “alkyl” group refers to an aliphatic hydrocarbon group. An alkyl group can be saturated or unsaturated, and can be straight-chain (linear), branched or cyclic. In some embodiments, an alkyl group is not cyclic. In some embodiments, an alkyl group contains 1-30, 6-30, 6-20 or 8-20 carbon atoms. A “substituted” alkyl group is substituted with one or more substituents. In some embodiments, the one or more substituents are independently selected from halogens, nitro, cyano, oxo, hydroxy, alkoxy, haloalkoxy, aryloxy, thiol, alkylthio, arylthio, alkylsulfoxide, arylsulfoxide, alkylsulfone, arylsulfone, amino, alkylamino, dialkylamino, arylamino, alkoyl, carboxyl, carboxylate, esters, amides, carbonates, carbamates, ureas, alkyl, haloalkyl, fluoroalkyl, aralkyl, alkyl chains containing an acyl group, heteroalkyl, heteroali-cyclic, aryl, alkoxyaryl, heteroaryl, hydrophobic natural compounds (e.g., steroids), and the like. In some embodiments, an alkyl group as a substituent is linear or branched Ci-C₆ alkyl, which can be called “lower alkyl”. Non-limiting examples of lower alkyl groups include methyl, ethyl, propyl (including n-propyl and isopropyl), butyl (including all isomeric forms, such as n-butyl, isobutyl, sec-butyl and /er/-butyl), pentyl (including all isomeric forms, such as n-pentyl), and hexyl (including all isomeric forms, such as n-hexyl). In some embodiments, an alkyl group is attached to the Na-atom of a residue (e.g., Tyr or Dmt) of a peptide. In certain embodiments, an N-alkyl group is straight or branched C1-C₁₀ alkyl, or aryl-substituted alkyl such as benzyl, phenylethyl or the like. One or two alkyl groups can be attached to the Na-atom of the N-terminal residue. In some embodiments, an alkyl group is a 1-alkyl group that is attached to the C-1 position of a saccharide (e.g., glucose) via a glycosidic bond (e.g., an O-, S-, N- or C-glycosidic bond). In some embodiments, such a 1-alkyl group is an unsubstituted or substituted C1-C30, C₆-C30, C₆-C₂₀ or C₈-C₂₀ alkyl group. In some embodiments, an alkyl group (e.g., a 1-alkyl group) is substituted with one or more (e.g., 2 or 3) groups independently selected from aryl, —OH, —OR¹, —SH, —SR¹, —NH₂, —NHR¹, —N(R¹)₂, oxo (═O), —C(═O)R², carboxyl (—CO₂H), carboxylate (—CO₂—), —C(═O)OR¹, —OC(═O)R³, —C(═O)N(R¹)₂, —NR⁴C(═O)R³, —OC(═O)OR⁵, —OC(═O)N(R¹)₂, —NR⁴C(═O)OR⁵, and —NR⁴C(═O)N(R¹)₂, wherein: R¹ at each occurrence independently is hydrogen, alkyl or aryl, or both occurrences of R¹ and the nitrogen atom to which they are connected form a heterocyclyl or heteroaryl ring; R² at each occurrence independently is alkyl, heterocyclyl, aryl or heteroaryl; R³ at each occurrence independently is hydrogen, alkyl, heterocyclyl, aryl or heteroaryl; R⁴ at each occurrence independently is hydrogen or alkyl; and, R⁵ at each occurrence independently is alkyl or aryl. In some embodiments, an alkyl group (e.g., a 1-alkyl group) is internally or/and terminally substituted with a carboxyl/carboxylate group, an aryl group or an —O-aryl group. In certain embodiments, an alkyl group (e.g., a 1-alkyl group) is substituted with a carboxyl or carboxylate group at the distal end of the alkyl group. In further embodiments, an alkyl group (e.g., a 1-alkyl group) is substituted with an aryl group at the distal end of the alkyl group. In other embodiments, an alkyl group (e.g., a 1-alkyl group) is substituted with an —O-aryl group at the distal end of the alkyl group. The terms “halogen”, “halide” and “halo” refer to fluoride, chloride, bromide and iodide. The term “acyl” refers to —C(═O)R, where R is an aliphatic group that can be saturated or unsaturated, and can be linear, branched or cyclic. In certain embodiments, R contains 1-20, 1-10 or 1-6 carbon atoms. An acyl group can optionally be substituted with one or more groups, such as halogens, oxo, hydroxyl, alkoxy, thiol, alkylthio, amino, alkylamino, dialkylamino, cycloalkyl, aryl, acyl, carboxyl, esters, amides, hydrophobic natural compounds (e.g., steroids), and the like. The terms “heterocyclyl” and “heterocyclic” refer to a monocyclic non-aromatic group or a multicyclic group that contains at least one non-aromatic ring, wherein at least one non-aromatic ring contains one or more heteroatoms independently selected from O, N and S. The non-aromatic ring containing one or more heteroatoms may be attached or fused to one or more saturated, partially unsaturated or aromatic rings. In certain embodiments, a heterocyclyl or heterocyclic group has from 3 to 15, or 3 to 12, or 3 to 10, or 3 to 8, or 3 to 6 ring atoms. Heterocyclyl or heterocyclic groups include without limitation aziridinyl, azetidinyl, pyrrolidinyl, piperidinyl, morpholinyl, piperazinyl, azepanyl, azocanyl, oxiranyl, oxetanyl, tetrahydrofuranyl (oxolanyl), tetrahydropyranyl, oxepanyl and oxocanyl. The term “aryl” refers to a monocyclic aromatic hydrocarbon group or a multicyclic group that contains at least one aromatic hydrocarbon ring. In certain embodiments, an aryl group has from 6 to 15, or 6 to 12, or 6 to 10 ring atoms. Aryl groups include without limitation phenyl, naphthalenyl (naphthyl), fluorenyl, azulenyl, anthryl, phenanthryl, biphenyl and terphenyl. The aromatic hydrocarbon ring of an aryl group may be attached or fused to one or more saturated, partially unsaturated or aromatic rings—e.g., dihydronaphthyl, indenyl, indanyl and tetrahydronaphthyl (tetralinyl). An aryl group can optionally be substituted with one or more (e.g., 2 or 3) substituents independently selected from halogens (including —F and —Cl), cyano, nitro, hydroxyl, alkoxy, thiol, alkylthio, alkylsulfoxide, alkylsulfone, amino, alkylamino, dialkylamino, alkyl, haloalkyl (including fluoroalkyl such as trifluoromethyl), acyl, carboxyl, esters, amides, and the like. The term “heteroaryl” refers to a monocyclic aromatic group or a multicyclic group that contains at least one aromatic ring, wherein at least one aromatic ring contains one or more heteroatoms independently selected from O, N and S. The heteroaromatic ring may be attached or fused to one or more saturated, partially unsaturated or aromatic rings that may contain only carbon atoms or that may contain one or more heteroatoms. In certain embodiments, a heteroaryl group has from 5 to 15, or 5 to 12, or 5 to 10 ring atoms. Monocyclic heteroaryl groups include without limitation pyrrolyl, pyrazolyl, pyrazolinyl, imidazolyl, oxazolyl, isoxazolyl, thiazolyl, thiadiazolyl, isothiazolyl, furanyl, thienyl (thiophenyl), oxadiazolyl, triazolyl, tetrazolyl, pyridyl, pyridonyl, pyrazinyl, pyrimidinyl, pyridazinyl, pyridazinonyl and triazinyl. Non-limiting examples of bicyclic heteroaryl groups include indolyl, benzothiazolyl, benzothiadiazolyl, benzoxazolyl, benzisoxazolyl, benzothienyl (benzothiophenyl), quinolinyl, tetrahydroisoquinolinyl, isoquinolinyl, benzimidazolyl, benzotriazolyl, indolizinyl, benzofuranyl, isobenzofuranyl, chromonyl, coumarinyl, cinnolinyl, quinazolinyl, quinoxalinyl, indazolyl, naphthyridinyl, phthalazinyl, quinazolinyl, purinyl, pyrrol opyridinyl, furopyridinyl, thienopyridinyl, dihydroisoindolyl and tetrahydroquinolinyl.

In some embodiments, for instance, the dual agonist peptides can be associated with a saccharide, such as within a pharmaceutically acceptable composition or lyophilizate. Saccharides include monosaccharides, disaccharides and oligosaccharides (e.g., trisaccharides, tetrasaccharides and so on). A reducing saccharide exists in a ring form and an open-chain form in equilibrium, which generally favors the ring form. A functionalized saccharide of a surfactant moiety has a functional group suitable for forming a stable covalent bond with an amino acid of a dual agonist peptide.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition. In one embodiment, a pharmaceutically acceptable composition in which a dual agonist peptide can be formulated comprises polysorbate 20 (e.g., about 0.050% (w/w)); optionally methylparaben (e.g., about 0.300% (w/w)); arginine (about 0.348% (w/w)), and mannitol (e.g., about 4.260% (w/w)) in distilled (DI) water.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician.

The terms “treat,” “treating” and “treatment” include alleviating, ameliorating, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition. The term “medical conditions” (or “conditions” for brevity) includes diseases and disorders. The terms “diseases” and “disorders” are used interchangeably herein.

The disclosure also provides pharmaceutical compositions comprising a dual agonist peptide product described herein or a pharmaceutically acceptable salt thereof, and one or more pharmaceutically acceptable carriers or excipients. A pharmaceutical composition contains a therapeutically effective amount of a peptide product or an appropriate fraction thereof. A composition can optionally contain an additional therapeutic agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)), and organic solvents (e.g., dimethyl sulfoxide and alcohols [e.g., ethanol, glycerol and propylene glycol]). Except insofar as any conventional excipient or carrier is incompatible with a peptide product, the disclosure encompasses the use of conventional excipients and carriers in formulations containing a peptide product. See, e.g., Remington: The Science and Practice of Pharmacy, 21st Ed., Lippincott Williams & Wilkins (Philadelphia, Pa.) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Fla.) (2004).

An appropriate or suitable formulation can depend on various factors, such as the route of administration chosen. Potential routes of administration of a pharmaceutical composition comprising a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository). In certain embodiments, a present dual agonist peptide product is administered parenterally (e.g., subcutaneously, intravenously or intramuscularly). In other embodiments, a peptide product is administered by oral inhalation or nasal inhalation or insufflation. In some embodiments, the carrier is an aqueous-based carrier, such as in a parenteral (e.g., subcutaneous, intravenous or intramuscular) formulation. In other embodiments, the carrier is a nonaqueous-based carrier. In certain embodiments, the nonaqueous-based carrier is a hydrofluoroalkane (HFA) or HFA-like solvent that may comprise sub-micron anhydrous a-lactose or/and other excipients, such as in a formulation for administration by oral inhalation or nasal inhalation or insufflation.

In some embodiments, a peptide product is administered parenterally (e.g., subcutaneously, intravenously or intramuscularly) by injection. Parenteral administration bypasses the strongly acidic environment of the stomach, gastrointestinal (GI) absorption and first-pass metabolism. Excipients and carriers that can be used to prepare parenteral formulations include without limitation solvents (e.g., aqueous solvents such as water, saline, physiological saline, buffered saline [e.g., PBS], balanced salt solutions [e.g., Ringer's BSS] and aqueous dextrose solutions), isotonic/iso-osmotic agents (e.g., salts [e.g., NaCl, KCl and CaCl₂] and sugars [e.g., sucrose]), buffering agents and pH adjusters (e.g., sodium dihydrogen phosphate [monobasic sodium phosphate]/di sodium hydrogen phosphate [dibasic sodium phosphate], citric acid/sodium citrate and L-histidine/L-histidine HC1), and emulsifiers (e.g., non-ionic surfactants such as polysorbates [e.g., polysorbate 20 and 80] and poloxamers [e.g., poloxamer 188]). Peptide formulations and delivery systems are discussed in, e.g., A. J. Banga, Therapeutic Peptides and Proteins: Formulation, Processing, and Delivery Systems, 3rd Ed., CRC Press (Boca Raton, Fla.) (2015). The excipients can optionally include one or more substances that increase peptide stability, increase peptide solubility, inhibit peptide aggregation or reduce solution viscosity, or any combination or all thereof. Such substances include without limitation hydrophilic amino acids (e.g., arginine and histidine), polyols (e.g., myo-inositol, mannitol and sorbitol), saccharides (e.g., glucose (including D-glucose [dextrose]), lactose, sucrose and trehalose}, osmolytes (e.g., trehalose, taurine, amino acids [e.g., glycine, sarcosine, alanine, proline, serine, b-alanine and g-aminobutyric acid], and betaines [e.g., trimethylglycine and trimethylamine N-oxide]), and non-ionic surfactants (e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™ F-68], and Genapol® PF-10 and variants thereof). Because such substances increase peptide solubility, they can be used to increase peptide concentration in a formulation. Higher peptide concentration in a formulation is particularly advantageous for subcutaneous administration, which has a limited volume of bolus administration (e.g., <about 1.5 mL). In addition, such substances can be used to stabilize peptides during the preparation, storage and reconstitution of lyophilized peptides. An exemplary parenteral formulation comprises a peptide product, mannitol, methionine, sodium thioglycolate, polysorbate 20, a pH adjuster (e.g., NaOH or/and HC1) and de-ionized water. Excipients of parenteral formulations that would be suitable for use with the dual agonist peptides described herein (e.g., various combinations of excipients including NaCl and the like) are well-known and available to those of ordinary skill in the art.

For parenteral (e.g., subcutaneous, intravenous or intramuscular) administration, a sterile solution or suspension of a peptide product in an aqueous solvent containing one or more excipients can be prepared beforehand and can be provided in, e.g., a pre-filled syringe of a single-use pen or a pen with a dose counter. Alternatively, a peptide product can be dissolved or suspended in an aqueous solvent that can optionally contain one or more excipients prior to lyophilization (freeze-drying). Shortly prior to parenteral administration, the lyophilized peptide product stored in a suitable container (e.g., a vial) can be reconstituted with, e.g., sterile water that can optionally contain one or more excipients. In other embodiments, an agonist peptide product is administered intranasally. The nasal mucosa provides a big surface area, a porous endothelium, a highly vascular subepithelial layer and a high absorption rate, and hence allows for high bioavailability. An intranasal formulation can comprise a peptide product along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or spray using, e.g., a metering atomizing spray pump. Table 2 shows exemplary excipients of nasal-spray formulations.

TABLE 2
Exemplary excipients and carriers
of nasal and pulmonary formulations

| Dosage | |
|---|---|
| Form | Ingredients in Addition to a Peptide Product |
| nasal | microcrystalline cellulose, sodium carboxymethylcellulose, |
| spray | dextrose, water, and optionally a pH adjuster (e.g., HCl) |
| nasal | microcrystalline cellulose, carboxymethyl |
| spray | cellulose sodium, dextrose, polysorbate 80, |
| | disodium edetate, potassium sorbate, a pH adjuster |
| | (e.g., HCl), water, and optionally an alcohol (e.g., ethanol) |
| nasal | microcrystalline cellulose, carboxymethyl cellulose sodium, |
| spray | dextrose, polysorbate 80, benzalkonium chloride, phenylethyl |
| | alcohol, water, and optionally an alcohol (e.g., ethanol) |
| nasal | hypromellose, benzalkonium chloride, NaCl, |
| spray | EDTA, citric acid, sodium phosphate dibasic, |
| | water, and optionally an alcohol (e.g., ethanol) |
| inhalation | mannitol, glycine, sodium citrate and NaOH |
| (DPI) | |
| inhalation | lactose, starch, a starch derivative (e.g., |
| (DPI) | hydroxypropylmethyl cellulose) or polyvinylpyrrolidine, |
| | and optionally magnesium stearate or/and leucine |
| inhalation | a propellant (e.g., 1,1,1,2-tetrafluoroethane), a surfactant |
| (MDI) | (e.g., lecithin or oleic acid), and a co-solvent (e.g., ethanol) |
| inhalation | polysorbate 80, edetate disodium, |
| (nebulizer) | sodium chloride, pH buffering agents |
| | (e.g., citric acid/sodium citrate), and water |

In further embodiments, a peptide product is administered via a pulmonary route, such as by oral inhalation or nasal inhalation. Pulmonary administration of a drug can treat a lung disorder or/and a systemic disorder, as the lungs serve as a portal to the systemic circulation. Advantages of pulmonary drug delivery include, for example: 1) avoidance of first-pass metabolism; 2) fast drug action; 3) large surface area of the alveolar region for absorption, high permeability of the lungs (thin air-blood barrier), and profuse vasculature of the airways; and 4) reduced extracellular enzyme levels compared to the GI tract due to the large alveolar surface area. An advantage of oral inhalation over nasal inhalation includes deeper penetration/deposition of the drug into the lungs, although nasal inhalation can deliver the drug into systemic circulation transmucosally in the nasal cavity as well as in the lungs. Oral or nasal inhalation can be achieved by means of, e.g., a metered-dose inhaler (MDI), a nebulizer or a dry powder inhaler (DPI). For example, a peptide product can be formulated for aerosol administration to the respiratory tract by oral or nasal inhalation. The drug is delivered in a small particle size (e.g., between about 0.5 micron and about 5 microns), which can be obtained by micronization, to improve, e.g., drug deposition in the lungs and drug suspension stability. The drug can be provided in a pressurized pack with a suitable propellant, such as a hydrofluoroalkane (HFA, e.g., 1,1,1,2-tetrafluoroethane [HFA-134a]), a chlorofluorocarbon (CFC, e.g., dichlorodifluoromethane, trichlorofluoromethane or dichlorotetrafluoroethane), or a suitable gas (e.g., oxygen, compressed air or carbon dioxide). The drug in the aerosol formulation is dissolved, or more often suspended, in the propellant for delivery to the lungs. The aerosol can contain excipients such as a surfactant (which enhances penetration into the lungs by reducing the high surface tension forces at the air-water interface within the alveoli, may also emulsify, solubilize or/and stabilize the drug, and can be, e.g., a phospholipid such as lecithin) or/and a stabilizer, although the surfactant moiety of the peptide product can perform functions of a surfactant. For example, an MDI formulation can comprise a peptide product, a propellant (e.g., an HFA such as 1,1,1,2-tetrafluoroethane) and a co-solvent (e.g., an alcohol such as ethanol), and optionally a surfactant (e.g., a fatty acid such as oleic acid). The MDI formulation can optionally contain a dissolved gas (e.g., C0₂). After device actuation, the bursting of C0₂ bubbles within the emitted aerosol droplets breaks up the droplets into smaller droplets, thereby increasing the respirable fraction of drug. As another example, a nebulizer formulation can comprise a peptide product, a chelator or preservative (e.g., edetate disodium), an isotonicity agent (e.g., NaCl), pH buffering agents (e.g., citric acid/sodium citrate) and water, and optionally a surfactant (e.g., a Tween® such as polysorbate 80). The drug can be delivered by means of, e.g., a nebulizer or an MDI with or without a spacer, and the drug dose delivered can be controlled by a metering chamber (nebulizer) or a metering valve (MDI).

Table 2 shows exemplary MDI, nebulizer and DPI formulations. Metered-dose inhalers (also called pressurized metered-dose inhalers [pMDI]) are the most widely used inhalation devices. A metering valve delivers a precise amount of aerosol (e.g., about 20-100 pL) each time the device is actuated. MDIs typically generate aerosol faster than the user can inhale, which can result in deposition of much of the aerosol in the mouth and the throat. The problem of poor coordination between device actuation and inhalation can be addressed by using, e.g., a breath-actuated MDI or a coordination device. A breath-actuated MDI (e.g., Easi Breathe®) is activated when the device senses the user's inspiration and discharges a drug dose in response. The inhalation flow rate is coordinated through the actuator and the user has time to actuate the device reliably during inhalation. In a coordination device, a spacer (or valved holding chamber), which is a tube attached to the mouthpiece end of the inhaler, serves as a reservoir or chamber holding the drug that is sprayed by the inhaler and reduces the speed at which the aerosol enters the mouth, thereby allowing for the evaporation of the propellant from larger droplets. The spacer simplifies use of the inhaler and increases the amount of drug deposited in the lungs instead of in the upper airways. The spacer can be made of an anti-static polymer to minimize electrostatic adherence of the emitted drug particles to the inner walls of the spacer. Nebulizers generate aerosol droplets of about 1-5 microns. They do not require user coordination between device actuation and inhalation, which can significantly affect the amount of drug deposited in the lungs. Compared to MDIs and DP Is, nebulizers can deliver larger doses of drug, albeit over a longer administration time. Examples of nebulizers include without limitation human-powered nebulizers, jet nebulizers (e.g., AeroEclipse® II BAN [breath-actuated], CompAIR™ NE-C801 [virtual valve], PARI LC® Plus [breath-enhanced] and SideStream Plus [breath-enhanced]), ultrasonic wave nebulizers, and vibrating mesh nebulizers (e.g., Akita2® Apixneb, I-neb AAD System with metering chambers, MicroAir® NE-U22, Omron U22 and PARI eFlow® rapid). As an example, a pulsed ultrasonic nebulizer can aerosolize a fixed amount of the drug per pulse, and can comprise an opto-acoustical trigger that allows the user to synchronize each breath to each pulse. For oral or nasal inhalation using a dry powder inhaler (DPI), a peptide product can be provided in the form of a dry micronized powder, where the drug particles are of a certain small size (e.g., between about 0.5 micron and about 5 microns) to improve, e.g., aerodynamic properties of the dispersed powder and drug deposition in the lungs. Particles between about 0.5 micron and about 5 microns deposit by sedimentation in the terminal bronchioles and the alveolar regions. By contrast, the majority of larger particles (>5 microns) do not follow the stream of air into the many bifurcations of the airways, but rather deposit by impaction in the upper airways, including the oropharyngeal region of the throat. A DPI formulation can contain the drug particles alone or be blended with a powder of a suitable larger base/carrier, such as lactose, starch, a starch derivative (e.g., hydroxypropylmethyl cellulose) or polyvinylpyrrolidine. The carrier particles enhance flow, reduce aggregation, improve dose uniformity and aid in dispersion of the drug particles. A DPI formulation can optionally contain an excipient such as magnesium stearate or/and leucine that improves the performance of the formulation by interfering with inter-particle bonding (by anti-adherent action). The powder formulation can be provided in unit dose form, such as a capsule (e.g., a gelatin capsule) or a cartridge in a blister pack, which can be manually loaded or pre-loaded in an inhaler. The drug particles can be drawn into the lungs by placing the mouthpiece or nosepiece of the inhaler into the mouth or nose, taking a sharp, deep inhalation to create turbulent airflow, and holding the breath for a period of time (e.g., about 5-10 seconds) to allow the drug particles to settle down in the bronchioles and the alveolar regions. When the user actuates the DPI and inhales, airflow through the device creates shear and turbulence, inspired air is introduced into the powder bed, and the static powder blend is fluidized and enters the user's airways. There, the drug particles separate from the carrier particles due to turbulence and are carried deep into the lungs, while the larger carrier particles impact on the oropharyngeal surfaces and are cleared. Thus, the user's inspiratory airflow achieves powder de-agglomeration and aeroionisation, and determines drug deposition in the lungs. (While a passive DPI requires rapid inspiratory airflow to de agglomerate drug particles, rapid inspiration is not recommended with an MDI or nebulizer, since it creates turbulent airflow and fast velocity which increase drug deposition by impaction in the upper airways.) Compared to an MDI, a DPI (including a breath-activated DPI) may be able to deliver larger doses of drug, and larger-size drugs (e.g., macromolecules), to the lungs.

Lactose (e.g., alpha-lactose monohydrate) is the most commonly used carrier in DPI formulations. Examples of grades/types of lactose monohydrate for DPI formulations include without limitation DCL 11, Flowlac® 100, Inhalac® 230, Lactohale® 300, Lactopress® SD 250 (spray-dried lactose), Respitose® SV003 and Sorbolac® 400. A DPI formulation can contain a single lactose grade or a combination of different lactose grades. For example, a fine lactose grade like Lactohale® 300 or Sorbolac® 400 may not be a suitable DPI carrier and may need to be blended with a coarse lactose grade like DCL 11, Flowlac® 100, Inhalac® 230 or Respitose® SV003 (e.g., about a 1:9 ratio of fine lactose to coarse lactose) to improve flow.

Tables 3 and 4 show non-limiting examples of grades/types of lactose that can be used in DPI formulations. The distribution of the carrier particle sizes affects the fine particle fraction/dose (FPF or FPD) of the drug, with a high FPF being desired for drug delivery to the lungs. FPF/FPD is the respirable fraction/dose mass out of the DPI device with an aerodynamic particle size <5 microns in the inspiration air. High FPF, and hence good DPI performance, can be obtained from, e.g., DPI formulations having an approximately 1:9 ratio of fine lactose (e.g., Lactohale® 300) to coarse lactose (e.g., Respitose® SV003) and about 20% w/w overages to avoid deposition of the drug in the capsule shell or the DPI device and to deliver essentially all of the drug to the airways.

TABLE 3

| | | Range of Particle Sizes (μm) | | |
|---|---|---|---|---|
| Product | Type | 10% | 50% | 90% |
| Lactohale ® | LH200 | <9 | <69 | <141 |
| InhaLac ® | 230 | <35 | <93 | <138 |
| Respitose ® | ML001 | <4 | <43 | <146 |
| | ML003 | <4 | <35 | <106 |
| | SV003 | <30 | <59 | <90 |
| | SV004 | <32 | <61 | <93 |

TABLE 4

| | | Range of Particle Sizes | | | |
|---|---|---|---|---|---|
| Product | Type | <45 μm | <100 μm | <150 μm | <250 μm |
| Respitose ® | ML003 | 65% | 98% | 100% | NA |
| Respitose ® | ML002 | 65% | 98% | NA | 100% |

Other carriers for DPI formulations include without limitation glucose, mannitol (e.g., crystallized mannitol [Pearlitol 110 C] and spray-dried mannitol [Pearlitol 100 SD]), maltitol (e.g., crystallized maltitol [Maltisorb P90]), sorbitol and xylitol. Most DPIs are breath-activated (“passive”), relying on the user's inhalation for aerosol generation. Examples of passive DPIs include without limitation Airmax®, Novolizer® and Otsuka DPI (compact cake). The air classifier technology (ACT) is an efficient passive powder dispersion mechanism employed in DPIs. In ACT, multiple supply channels generate a tangential airflow that results in a cyclone within the device during inhalation. There are also power-assisted (“active”) DPIs (based on, e.g., pneumatics, impact force or vibration) that use energy to aid, e.g., particle de-agglomeration. For example, the active mechanism of Exubera® inhalers utilizes mechanical energy stored in springs or compressed-air chambers. Examples of active DPIs include without limitation Actispire® (single-unit dose), Aspirair® (multi-dose), Exubera® (single-unit dose), MicroDose® (multi-unit dose and electronically activated), Omnihaler® (single-unit dose), Pfeiffer DPI (single-unit dose), and Spiros® (multi-unit dose). A peptide product can also be administered by other routes, such as orally. An oral formulation can contain a peptide product and conventional excipients known in the art, and optionally an absorption enhancer such as sodium V-[8-(2-hydroxybenzoyl) aminocaprylate] (SNAC). SNAC protects against enzymatic degradation via local buffering action and enhances GI absorption. An oral dosage form (e.g., a tablet, capsule or pill) can optionally have an enteric coating to protect its content from the strong acids and proteolytic enzymes of the stomach. In some embodiments, a peptide product is delivered from a sustained-release composition. As used herein, the term “sustained-release composition” encompasses sustained-release, prolonged-release, extended-release, delayed-release, slow-release and controlled-release compositions, systems and devices. In some embodiments, a sustained-release composition delivers a peptide product over a period of at least about 1 week, 2 weeks, 3 weeks, 1 month, 2 months, 3 months or longer. In some embodiments, a sustained-release composition is formulated as nanoparticles or microparticles composed of a biodegradable polymer and incorporating a peptide product. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. In further embodiments, a sustained-release composition is in the form of a depot that is generated when a mixture of a peptide product and a polymer is injected into a subject intramuscularly or subcutaneously. In certain embodiments, the polymer is or comprises PEG, polylactic acid (PLA) or polyglycolic acid (PGA), or a copolymer thereof (e.g., PLGA or PLA-PEG).

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug, but can contain an appropriate fraction thereof so that taking multiple unit dosage forms achieves the therapeutically effective dose. Examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; and a capsule, cartridge or blister pre-loaded in or manually loaded into an inhaler. Alternatively, a pharmaceutical composition can be presented as a kit in which the active ingredient, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected parenterally). A kit can contain all active and inactive ingredients in unit dosage form or the active ingredient and inactive ingredients in two or more separate containers, and can contain instructions for administering or using the pharmaceutical composition to treat a medical condition disclosed herein. A kit can further contain a device for delivering the composition, such as an injection pen or an inhaler. In some embodiments, a kit contains a peptide product or a pharmaceutically acceptable salt thereof, or a pharmaceutical composition comprising the same, and instructions for administering or using the peptide product or the composition to treat a medical condition disclosed herein, such as insulin resistance, diabetes, obesity, metabolic syndrome or a cardiovascular disease, or a condition associated therewith (e.g., NASH or PCOS). In certain embodiments, the kit further contains a device for delivering the peptide product or the composition, such as an injection pen or an inhaler.

The disclosure further provides uses of the dual agonist peptide products described herein to prevent and/or treat conditions associated with GLP1R and/or GCGR, such as but not limited to insulin resistance, diabetes, obesity, metabolic syndrome and cardiovascular diseases, and conditions associated therewith, such as NASH and PCOS. In some embodiments, the dual agonist peptide products can be used to treat hyperglycemia, insulin resistance, hyperinsulinemia, prediabetes, diabetes (including types 1 and 2, gestational and juvenile diabetes), diabetic complications, diabetic neuropathy, diabetic nephropathy, diabetic retinopathy, hyperlipidemia, hypercholesterolemia, hypertriglyceridemia, elevated blood levels of free fatty acids, obesity, metabolic syndrome, syndrome X, cardiovascular diseases (including coronary artery disease), atherosclerosis, acute cardiovascular syndrome, ischemia (including myocardial ischemia and cerebral ischemia/stroke), ischemia-reperfusion injury (including myocardial and cerebral IRI), infarction (including myocardial and cerebral infarction), angina, heart failure (e.g., congestive heart failure), peripheral vascular disease, thrombosis (e.g., deep vein thrombosis), embolism (e.g., pulmonary embolism), systemic inflammation (e.g., one characterized by elevated C-reactive protein blood level), and hypertension. The dual agonist peptide products can achieve their therapeutic effects through various mechanisms, including stimulation of blood glucose-dependent insulin secretion, increase in insulin sensitivity, stimulation of fat burning and reduction of body weight. The dual agonist peptide products can also promote, e.g., pancreatic beta-cell protection, cardioprotection and wound healing.

The peptide products described herein can be used to treat other conditions associated with insulin resistance or/and obesity. Other conditions associated with insulin resistance or/and obesity include without limitation arthritis (e.g., osteoarthritis), low back pain, breathing disorders (e.g., asthma, obesity hypoventilation syndrome [Pickwickian syndrome] and obstructive sleep apnea), dermatological disorders (e.g., diabetic ulcers, acanthosis nigricans, cellulitis, hirsutism, intertrigo and lymphedema), gastroenterological disorders (e.g., cholelithiasis [gallstone], gastroesophageal reflux disease [GERD] and gastroparesis), gout, hypercortisolism (e.g., Cushing's syndrome), kidney disorders (e.g., chronic kidney disease), liver disorders (e.g., fatty liver disease [FLD] including alcoholic and non-alcoholic FLD), neurological disorders (e.g., carpal tunnel syndrome, dementias [e.g., Alzheimer's disease and vascular dementia], meralgia paresthetica, migraines and multiple sclerosis), urological disorders (e.g., erectile dysfunction, hypogonadism and urinary incontinence), polycystic ovary syndrome, infertility, menstrual disorders, mood disorders (e.g., depression), and cancers (e.g., cancers of the endometrium, esophagus, colorectum, gallbladder, kidney, liver [e.g., hepatocellular carcinoma], pancreas and skin [e.g., melanoma], and leukemia). In certain embodiments, a dual agonist peptide product described herein is used to treat polycystic ovary syndrome (PCOS). In other embodiments, a peptide product is used to treat chronic kidney disease (CKD), also known as chronic kidney/renal failure (CKF/CRF). The most common causes of CKD are diabetes and long-term, uncontrolled hypertension. In further embodiments, a dual agonist peptide product described herein is used to treat fatty liver disease (FLD). In some embodiments, the FLD is non-alcoholic fatty liver disease (NAFLD). In certain embodiments, the NAFLD is non-alcoholic steatohepatitis (NASH). FLD, also known as hepatic steatosis, is characterized by excessive fat accumulation in the liver. FLD includes alcoholic fatty liver disease (AFLD) and NAFLD. Chronic alcoholism causes fatty liver due to production of toxic metabolites such as aldehydes during metabolism of alcohol in the liver. NAFLD is described below. FLD is associated with diabetes, obesity and metabolic syndrome. Fatty liver can develop into cirrhosis or a liver cancer (e.g., hepatocellular carcinoma [HCC]). Less than about 10% of people with cirrhotic AFLD develop HCC, but up to about 45% of people with NASH without cirrhosis may develop HCC. HCC is the most common type of primary liver cancer in adults and occurs in the setting of chronic liver inflammation. NAFLD is characterized by fatty liver that occurs when fat, in particular free fatty acids and triglycerides, accumulates in liver cells (hepatic steatosis) due to causes other than excessive alcohol consumption, such as nutrient overload, high caloric intake and metabolic dysfunction (e.g., dyslipidemia and impaired glucose control). A liver can remain fatty without disturbing liver function, but a fatty liver can progress to become NASH, a condition in which steatosis is accompanied by inflammation, hepatocyte ballooning and cell injury with or without fibrosis of the liver. Fibrosis is the strongest predictor of mortality from NASH. NAFLD can be characterized by steatosis alone; steatosis with lobular or portal inflammation but without ballooning; steatosis with ballooning but without inflammation; or steatosis with inflammation and ballooning. NASH is the most extreme form of NAFLD. NASH is a progressive disease, with about 20% of patients developing cirrhosis of the liver and about 10% dying from a liver disease, such as cirrhosis or a liver cancer (e.g., HCC). NAFLD is the most common liver disorder in developed countries, and NASH is projected to supplant hepatitis C as the major cause of liver transplant in the U.S. by 2020. About 12-25% of people in the U.S. have NAFLD, with NASH affecting about 2-5% of people in the U.S. NAFLD, including NASH, is associated with insulin resistance, obesity and metabolic syndrome. For instance, insulin resistance contributes to progression of fatty liver to hepatic inflammation and fibrosis and thus NASH. Furthermore, obesity drives and exacerbates NASH, and weight loss can alleviate NASH. Therefore, the peptide products described herein, including GLP-1 receptor (GLP1R) agonists, glucagon receptor (GCGR) agonists and dual GLP1R/GCGR agonists, can be used to treat NAFLD, including NASH. In some embodiments, the dual agonist peptide products used to treat a condition associated with insulin resistance or/and obesity disclosed herein, such as NAFLD (e.g., NASH) or PCOS, are selected from the dual agonist peptide products of SEQ. ID. NOs. 1-10 or 12-27, and/or derivatives thereof, and pharmaceutically acceptable salts thereof.

In some embodiments, the present dual agonist peptide(s) can be used to control blood glucose with reduction of one or more adverse events (i.e., an unexpected event that negatively impacts patient and/or animal welfare) as compared to an agonist with unbalanced affinity for GU-LP. and GCGR (e.g., semaglutide). Exemplary, non-limiting adverse events can include nausea, vomiting, diarrhea, abdominal pain and/or constipation. Adverse events may also include any known to those of ordinary skill in the art, such as those listed in industry resources and/or otherwise known to those of ordinary skill in the art (see, e.g. Medical Dictionary for Regulatory Activities (MedDRA) (Pharm., Med. Transl. Med. 2018) and/or Clark, M. J. Biomed. Inf., 54, April 2015, pp. 167-173). Such adverse events can be determined in humans using standard techniques as are typically used in clinical trials (e.g., doctor visit, surveys/questionnaires). As compared to the frequency and/or severity of such an adverse event that occurs upon administration of an agonist with unbalanced affinity for GLP-1R and GCGR (e.g., semaglutide) to a subject, the dual agonist peptides of this disclosure (e.g., any of SEQ ID NOS. 1-10 or 12-27, or derivatives thereof) can decrease such frequency and/or severity thereof by, e.g., 20%, 40%, 50%, 60%, 70%, 80%, 90% of higher (up to 100%). In some embodiments, the dual agonist peptides of this disclosure (e.g., any of SEQ ID NOS. 1-10 or 12-27, or derivatives thereof) do not cause any adverse events.

A present dual agonist peptide product can be administered by any suitable route for treatment of a condition disclosed herein. Potential routes of administration of a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository)). In some embodiments, a peptide product is administered parenterally, such as subcutaneously, intravenously or intramuscularly. In other embodiments, a peptide product is administered by oral inhalation or nasal inhalation or insufflation. The therapeutically effective amount and the frequency of administration of, and the length of treatment with, a peptide product to treat a condition disclosed herein may depend on various factors, including the nature and severity of the condition, the potency of the compound, the route of administration, the age, body weight, general health, gender and diet of the subject, and the response of the subject to the treatment, and can be determined by the treating physician. In some embodiments, a peptide product is administered parenterally (e.g., subcutaneously (sc), intravenously (iv) or intramuscularly (im)) in a dose from about 0.01 mg to about 0.1, 1, 5 or 10 mg, or about 0.1-1 mg or 1-27 mg, over a period of about one week for treatment of a condition disclosed herein (e.g., one associated with insulin resistance or/and obesity, such as NASH or PCOS). In further embodiments, a peptide product is administered parenterally (e.g., sc, iv or im) in a dose of about 0.1-0.5 mg, 0.5-1 mg, 1-5 mg or 5-10 mg over a period of about one week. In certain embodiments, a peptide product is administered parenterally (e.g., subcutaneously (SC), intravenous (IV) or intramuscular (IM)) in a dose of about 0.1-1 mg, or about 0.1-0.5 mg or 0.5-1 mg, over a period of about one week. One of skill in the art understands that an effective dose in a mouse, or other pre-clinical animal model, may be scaled for a human. In that way, through allometric scaling (also referred to as biological scaling) a dose in a larger animal may be extrapolated from a dose in a mouse to obtain an equivalent dose based on body weight or body surface area of the animal.

A peptide product can be administered in any suitable frequency for treatment of a condition disclosed herein (e.g., one associated with insulin resistance or/and obesity, such as NASH or PCOS). In some embodiments, a dual agonist peptide product is administered, e.g., sc or iv once a day, once every two days, once every three days, twice a week, once a week or once every two weeks. In certain embodiments, a peptide product is administered, e.g., SC, IV, or IM once a week. A dual agonist peptide product can be administered at any time of day convenient to the patient. A dual agonist peptide product can be taken substantially with food (e.g., with a meal or within about 1 hour or 30 minutes before or after a meal) or substantially without food (e.g., at least about 1 or 2 hours before or after a meal). The length of treatment of a medical condition with a dual agonist peptide product can be based on, e.g., the nature and severity of the condition and the response of the subject to the treatment, and can be determined by the treating physician. In some embodiments, a dual agonist peptide product is administered chronically to treat a condition disclosed herein, such as at least about 2 months, 3 months, 6 months, 1 year, 1.5 years, 2 years, 3 years, 5 years, 10 years or longer. A dual agonist peptide product can also be taken pro re nata (as needed) until clinical manifestations of the condition disappear or clinical targets are achieved, such as blood glucose level, blood pressure, blood levels of lipids, body weight or body mass index, waist-to-hip ratio or percent body fat, or any combination thereof. If clinical manifestations of the condition re-appear or the clinical targets are not maintained, administration of the dual agonist peptide product can resume. The disclosure provides a method of treating a medical condition described herein, comprising administering to a subject in need of treatment a therapeutically effective amount of a peptide product described herein or a pharmaceutically acceptable salt thereof, or a pharmaceutical composition comprising the same. The disclosure further provides a peptide product described herein or a pharmaceutically acceptable salt thereof, or a composition comprising the same, for use as a medicament. In addition, the disclosure provides for the use of a peptide product described herein or a pharmaceutically acceptable salt thereof in the preparation of a medicament. The medicament containing the peptide product can be used to treat any medical condition described herein. The peptide product can optionally be used in combination with one or more additional therapeutic agents.

A dual agonist peptide product described herein can be administered as the sole active agent, or optionally be used in combination with one or more other dual agonist peptide products, and/or additional therapeutic agents to treat any disorder disclosed herein, such as insulin resistance, diabetes, obesity, metabolic syndrome or a cardiovascular disease, or any condition associated therewith, such as NASH or PCOS. In some embodiments, the one or more additional therapeutic agents are selected from antidiabetic agents, anti-obesity agents (including lipid-lowering agents and pro-satiety agents), anti-atherosclerotic agents, anti-inflammatory agents, antioxidants, antifibrotic agents, anti-hypertensive agents, and combinations thereof. Antidiabetic agents include without limitation: AMP-activated protein kinase (AMPK) agonists, including biguanides (e g., buformin and metformin); peroxisome proliferator-activated receptor gamma (PPAR-γ) agonists, including thiazolidinediones (e.g., balaglitazone, ciglitazone, darglitazone, englitazone, lobeglitazone, netoglitazone, pioglitazone, rivoglitazone, rosiglitazone and troglitazone), MSDC-0602K and saroglitazar (dual PPAR-α/γ agonist); glucagon-like peptide-1 (GLP-1) receptor agonists, including exendin-4, albiglutide, dulaglutide, exenatide, liraglutide, lixisenatide, semaglutide, taspoglutide, CNT0736, CNT03649, HM11260C (LAPS-Exendin), NN9926 (OG9S7GT), TT401 and ZYOG1; dipeptidyl peptidase 4 (DPP-4) inhibitors, including alogliptin, anagliptin, dutogliptin, evogliptin, gemigliptin, gosogliptin, linagliptin, omarigliptin, saxagliptin, septagliptin, sitagliptin, teneligliptin, trelagliptin and vildagliptin; sodium-glucose transport protein 2 (SGLT2) inhibitors, including canagliflozin (also inhibits SGLT1), dapagliflozin, empagliflozin, ertugliflozin, ipragliflozin, luseogliflozin, remogliflozin etabonate, sotagliflozin (also inhibits SGLT1) and tofogliflozin; blockers of ATP-dependent K⁺ (KA_{TP}) channels on pancreatic beta cells, including rneglitinides (e.g., mitiglinide, nateglinide and repagiinide) and sulfonylureas (including first generation (e.g., acetohexamide, carbutamide, chlorpropamide, giycyclamide [tolhexamide], metahexamide, tolazamide and tolbutamide) and second generation (e.g., glibenclamide, glyburide, glibornuride, gliclazide, glimepiride, glipizide, gliquidone, glisoxepide and glyclopyramide); insulin and analogs thereof, including fast-acting insulin (e.g., insulin aspari insulin glulisine and insulin lispro), intermediate-acting insulin (e.g., NPH insulin), and long-acting insulin (e.g., insulin degludec, insulin detemir and insulin glargine); and/or, analogs, derivatives and salts thereof. In certain embodiments, the antidiabetic agent is or includes a biguanide (e.g., metformin), a thiazolidinedione (e.g., pioglitazone or rosiglitazone) or a SGLT2 inhibitor (e.g., empagliflozin or tofogliflozin), or any combination thereof. Anti-obesity agents include, but are not limited to: appetite suppressants (anorectics), including amphetamine, dexamphetamine, amfepramone, clobenzorex, mazindol, phentermine (with or without topiramate) and lorcaserin; pro-satiety agents, including ciliary neurotrophic factor (e.g., axokine) and longer-acting analogs of amylin, calcitonin, cholecystokinin (CCK), GLP-1, leptin, oxyntomodulin, pancreatic polypeptide (PP), peptide YY (PYY) and neuropeptide Y (NPY); lipase inhibitors, including caulerpenyne, cetilistat, ebelactone A and B, esterastin, lipstatin, orlistat, percyquinin, panclicin A-E, valilactone and vibralactone; antihyperlipidemic agents; and analogs, derivatives and salts thereof. Antihyperlipidemic agents include without limitation: HMG-CoA reductase inhibitors, including statins {e.g., atorvastatin, cerivastatin, fluvastatin, mevastatin, monacolins (e.g., monacolin K (lovastatin), pitavastatin, pravastatin, rosuvastatin and simvastatin} and flavanones (e.g., naringenin); squalene synthase inhibitors, including lapaquistat, zaragozic acid and RPR-107393; acetyl-CoA carboxylase (ACC) inhibitors, including anthocyanins, avenaciolides, chloroacetylated biotin, cyclodim, diclofop, haloxyfop, soraphens (e.g., soraphen Aₗₐ), 5-(tetradecyloxy)-2-furancarboxylic acid (TOFA), CP-640186, GS-0976, NDI-010976; 7-(4-propyloxy-phenylethynyl)-3,3-dimethyl-3,4dihydro-2H-benzo[b] [1,4]dioxepine; N-ethyl-N′-(3-{[4-(3,3-dimethyl-1-oxo-2-oxa-7-azaspiro[4.5]dec-7-yl)piperidin-1-yl]-carbonyl}-1-benzothien-2-yl)urea; 5-(3-acetamidobut-1-ynyl)-2-(4-propyloxyphenoxy)thiazole; and 1434 [4-(3,3-dimethyl-1-oxo-2-oxa-7-azaspiro[4.5]dec-7-yl)piperidin-1-yl]-carbonyl-5-(pyridin-2-yl)-2-thienyl)-3-ethylurea; PPAR-α agonists, including fibrates (e.g., bezafibrate, ciprofibrate, clinofibrate, clofibric acid, clofibrate, aluminum clofibrate [alfibrate], clofibride, etofibrate, fenofibric acid, fenofibrate, gemfibrozil, ronifibrate and simfibrate), isoflavones (e.g., daidzein and genistein), and perfluoroalkanoic acids (e.g., perfluorooctanoic acid and perfluorononanoic acid); PPAR-δ agonists, including elafibranor (dual PPAR-α/γ agonist), GFT505 (dual PPAR-α/γ agonist), GW0742, GW501516 (dual PPAR-β/δ agonist), sodelglitazar (GW677954), MBX-8025, and isoflavones (e.g., daidzein and genistein); PPAR-γ agonists, including thiazolidinediones {supra), saroglitazar (dual PPAR-α/γ agonist), 4-oxo-2-thioxothiazolines (e.g., rhodanine), berberine, honokiol, perfluorononanoic acid, cyclopentenone prostaglandins (e.g., cyclopentenone 15-deoxy-A-prostaglandin J₂ [15d-PGJ₂]), and isoflavones (e.g., daidzein and genistein); liver X receptor (LXR) agonists, including endogenous ligands (e.g., oxysterols such as 22(i?)-hydroxycholesterol, 24(A)-hydroxy cholesterol, 27-hydroxycholesterol and cholestenoic acid) and synthetic agonists (e.g., acetyl-podocarpic dimer, hypocholamide, A(X-di methyl-3b-hydroxy-cholenamide [DMHCA], GW3965 and T0901317); retinoid X receptor (RXR) agonists, including endogenous ligands (e.g., 9-cis-retinoic acid) and synthetic agonists (e.g., bexarotene, AGN 191659, AGN 191701, AGN 192849, BMS649, LG100268, LG100754 and LGD346); inhibitors of acyl-CoA cholesterol acyltransferase (ACAT, aka sterol G-acyl transferase [SOAT], including ACAT1 [SOAT1] and ACAT2 [SOAT2]), including avasimibe, pactimibe, pellitorine, terpendole C and flavanones (e.g., naringenin); inhibitors of stearoyl-CoA desaturase-1 (SCD-1, aka stearoyl-CoA delta-9 desaturase) activity or expression, including aramchol, CAY-10566, CVT-11127, SAR-224, SAR-707, XEN-103; 3-(2-hydroxyethoxy)-4-methoxy-N-[5-(3-trifluoromethylbenzyl)thiazol-2-yl]benzamide and 4-ethyl amino-3-(2-hydroxyethoxy)-N-[5-(3-trifluoromethylbenzyl)thiazol-2-yl]benzamide; 1′{6-[5-(pyridin-3-ylmethyl)-1,3,4-oxadiazol-2-yl]pyridazin-3-yl}-5-(trifluoromethyl)-3,4-dihydrospiro[chromene-2,4′-piperidine]; 5-fluoro-1′-{6-[5-(pyridin-3-ylmethyl)-1,3,4-oxadiazol-2-yl]pyridazin-3-yl}-3,4-dihydrospiro[chromene-2,4′-piperidine]; 6-[5-(cyclopropylmethyl)-4,5-dihydro-1′H,3H-spiro[1,5-benzoxazepine-2,4′-piperidin]-1′-yl]-N-(2-hydroxy-2-pyridin-3-ylethyl)pyridazine-3-carboxamide; 6-[4-(2-methylbenzoyl)piperidin-1-yl]pyridazine-3-carboxylic acid (2-hydroxy-2-pyridin-3-ylethyl)amide; 4-(2-chlorophenoxy)-N-[3-(methyl carbamoyl)phenyl]piperidine-1-carboxamide; the cis-9,trans-11 isomer and the trans-10, cis-12 isomer of conjugated linoleic acid, substituted heteroaromatic compounds disclosed in WO 2009/129625 A1, anti-sense polynucleotides and peptide-nucleic acids (PNAs) that target mRNA for SCD-1, and SCD-1-targeting siRNAs; cholesterylester transfer protein (CETP) inhibitors, including anacetrapib, dalcetrapib, evacetrapib, torcetrapib and AMG 899 (TA-8995); inhibitors of microsomal triglyceride transfer protein (MTTP) activity or expression, including implitapide, lomitapide, dirlotapide, mitratapide, CP-346086, JTT-130, SLx-4090, anti-sense polynucleotides and PNAs that target mRNA for MTTP, MTTP-targeting microRNAs (e.g., miRNA-30c), and MTTP-targeting siRNAs; GLP-1 receptor agonists; fibroblast growth factor 21 (FGF21) and analogs and derivatives thereof, including BMS-986036 (pegylated FGF21); inhibitors of pro-protein eonvertase subtilisin/kexin type 9 (PCSK9) activity or expression, including berberine (reduces PC8K9 level), annexin A2 (inhibits PCSK9 activity), anti-PC SK9 antibodies (e.g., alirocumab, bococizumab, evolocumab, LGT-209, LY3015014 and RG7652), peptides that mimic the epidermal growth factor-A (EGF-A) domain of the LDL receptor which binds to PCSK9, PCSK9-binding adnectins (e.g., BMS-962476), anti-sense polynucleotides and PNAs that target mRNA for PCSK9, and PCSK9-targeting siRNAs (e.g, inclisiran [ALN-PCS] and ALN-PCS02); apolipoprotein mimetic peptides, including apoA-I mimetics (e.g., 2F, 3F, 3F-1, 3F-2, 3F-14, 4F, 4F-P-4F, 4F-IHS-4F, 4F2, 5F, 6F, 7F, 18F, 5A, 5A-C1, 5A-CH1, 5A-CH2, 5A-H1, 18 A, 37 pA [18A-P-18A], ELK, ELK-1A, ELK-1F, ELK-1K1A1E, ELK-1L1K, ELK-1W, ELK-2A, ELK-2A2K2E, ELK-2E2K, ELK-2F, ELK-3 E3EK, ELK-3E3K3A, ELK-3E3LK, ELK-PA, ELK-P2A, ELKA, ELKA-CH2, ATI-5261, CS-6253, ETC-642, FAMP, FREL and KRES and apoE mimetics (e.g., Ac-hE18A-NH₂, AEM-28, Ac-[R]hE1 8 A-NH₂, AEM-28-14, EpK, hEp, mR18L, COG-112, COG-133 and COG-1410); omega-3 fatty acids, including docosahexaenoic acid (DHA), docosapentaenoic acid (DPA), eicosapentaenoic acid (EPA), a-linolenic acid (ALA), fish oils (which contain, e.g., DHA and EPA), and esters (e.g., glyceryl and ethyl esters) thereof; and analogs, derivatives and salts thereof. In certain embodiments, the anti-obesity agent is or includes a lipase inhibitor (e.g., orlistat) or/and an antihyperlipidemic agent (e.g., a statin such as atorvastatin, or/and a fibrate such as fenofibrate). Antihypertensive agents include without limitation: antagonists of the renin-angiotensin-aldosterone system (RAAS), including renin inhibitors (e.g., aliskiren), angiotensin-converting enzyme (ACE) inhibitors (e.g., benazepril, captopril, enalapril, fosinopril, lisinopril, moexipril, perindopril, quinapril, ramipril and trandolapril), angiotensin II receptor type 1 (ATII1) antagonists (e.g., azilsartan, candesartan, eprosartan, fimasartan, irbesartan, losartan, olmesartan medoxomil, olmesartan, telmisartan and valsartan), and aldosterone receptor antagonists (e.g., eplerenone and spironolactone); diuretics, including loop diuretics (e.g., bumetanide, ethacrynic acid, furosemide and torsemide), thiazide diuretics (e.g., bendroflumethiazide, chlorothiazide, hydrochlorothiazide, epitizide, methyclothi azide and polythiazide), thiazide-like diuretics (e.g., chlorthalidone, indapamide and metolazone), cicletanine (an early distal tubular diuretic), potassium-sparing diuretics (e.g., amiloride, eplerenone, spironolactone and triamterene), and theobromine; calcium channel blockers, including dihydropyridines (e.g., amlodipine, levamlodipine, cilnidipine, clevidipine, felodipine, isradipine, lercanidipine, nicardipine, nifedipine, nimodipine, nisoldipine and nitrendipine) and non-dihydropyri dines (e.g., diltiazem and verapamil); α₂-adrenoreceptor agonists, including clonidine, guanabenz, guanfacine, methyldopa and moxonidine; α1-adrenoreceptor antagonists (alpha blockers), including doxazosin, indoramin, nicergoline, phenoxybenzamine, phentolamine, prazosin, terazosin and tolazoline; β-adrenoreceptor (β1 or/and β₂) antagonists (beta blockers), including atenolol, betaxolol, bisoprolol, carteolol, carvedilol, labetalol, metoprolol, nadolol, nebivolol, oxprenolol, penbutolol, pindolol, propranolol and timolol; mixed alpha/beta blockers, including bucindolol, carvedilol and labetalol; endothelin receptor antagonists, including selective ETA receptor antagonists (e.g., ambrisentan, atrasentan, edonentan, sitaxentan, zibotentan and BQ-123) and dual ET_{A}/ET_{B} antagonists (e.g., bosentan, macitentan and tezosentan); other vasodilators, including hydralazine, minoxidil, theobromine, sodium nitroprusside, organic nitrates (e.g., isosorbide mononitrate, isosorbide dinitrate and nitroglycerin, which are converted to nitric oxide in the body), endothelial nitric oxide synthase (eNOS) stimulators (e.g., cicletanine), activators of soluble guanylate cyclase (e.g., cinaciguat and riociguat), phosphodiesterase type 5 (PDE5) inhibitors (e.g., avanafil, benzamidenafil, dasantafil, dynafil, lodenafil, mirodenafil, sildenafil, tadalafil, udenafil, vardenafil, dipyridamole, papaverine, propentofylline, zaprinast and T-1032), prostaglandin Ei (alprostadil) and analogs thereof (e.g., limaprost and misoprostol), prostacyclin and analogs thereof (e.g., ataprost, beraprost [e.g., esuberaprost], 5,6,7-trinor-4,8-inter-w-phenylene-9-fluoro-PGl₂, carbacyclin, isocarbacyclin, clinprost, ciprostene, eptaloprost, cicaprost, iloprost, pimilprost, SM-10906 (des-methyl pimilprost), naxaprostene, taprostene, treprostinil, CS-570, OP-2507 and TY-11223), non prostanoid prostacyclin receptor agonists (e.g., 1-phthalazinol, ralinepag, selexipag, ACT-333679 [MRE-269, active metabolite of selexipag], and TRA-418), phospholipase C (PLC) inhibitors, and protein kinase C (PKC) inhibitors (e.g., BIM-1, BIM-2, BIM-3, BIM-8, chelerythrine, cicletanine, gossypol, miyabenol C, myricitrin, ruboxistaurin and verbascoside; minerals, including magnesium and magnesium sulfate; and analogs, derivatives and salts thereof. In certain embodiments, the antihypertensive agent is or includes a thiazide or thiazide like diuretic (e.g., hydrochlorothiazide or chlorthalidone), a calcium channel blocker (e.g., amlodipine or nifedipine), an ACE inhibitor (e.g., benazepril, captopril or perindopril) or an angiotensin II receptor antagonist (e.g., olmesartan medoxomil, olmesartan, telmisartan or valsartan), or any combination thereof. In some embodiments, a peptide product described herein is used in combination with one or more additional therapeutic agents to treat NAFLD, such as NASH. In some embodiments, the one or more additional therapeutic agents are selected from antidiabetic agents, anti-obesity agents, anti-inflammatory agents, antifibrotic agents, antioxidants, anti hypertensive agents, and combinations thereof. Therapeutic agents that can be used to treat NAFLD (e.g., NASH) include without limitation: PPAR agonists, including PPAR-δ agonists (e.g., MBX-8025, elafibranor [dual PPAR-α/δ agonist] and GW501516 [dual PPAR-β/δ agonist]) and PPAR-γ agonists (e.g., thiazolidinediones such as pioglitazone, and saroglitazar [dual PPAR-α/γ agonist])-PPAR-δ and -γ agonism increases insulin sensitivity, PPAR-α agonism reduces liver steatosis and PPAR-δ agonism inhibits activation of macrophages and Kupffer cells; farnesoid X receptor (FXR) agonists, such as obeticholic acid and nonsteroidal FXR agonists like GS-9674 reduce liver gluconeogenesis, lipogenesis, steatosis and fibrosis; fibroblast growth factor 19 (FGF19) and analogs and derivatives thereof, such as NGM-282-FGF19 analogs reduce liver gluconeogenesis and steatosis; fibroblast growth factor 21 (FGF21) and analogs and derivatives thereof, such as BMS-986036 (pegylated FGF21)-FGF21 analogs reduce liver steatosis, cell injury and fibrosis; HMG-CoA reductase inhibitors, including statins (e.g., rosuvastatin)-statins reduce steatohepatitis and fibrosis; ACC inhibitors, such as NDI-010976 (liver-targeted) and GS-0976-ACC inhibitors reduce de novo lipogenesis and liver steatosis; SCD-1 inhibitors, such as aramchol-SCD-1 inhibitors reduce liver steatosis and increase insulin sensitivity; SGLT2 inhibitors, such as canagliflozin, ipragliflozin and luseogliflozin-SGLT2 inhibitors reduce body weight, liver ALT level and fibrosis; antagonists of CCR2 or/and CCR5, such as cenicriviroc-antagonists of CCR2 (binds to CCL2 [MCP1]) and CCR5 (binds to CCL5 [RANTES]) inhibit activation and migration of inflammatory cells (e.g., macrophages) to the liver and reduce liver fibrosis; apoptosis inhibitors, including apoptosis signal-regulating kinase 1 (ASK1) inhibitors (e.g., selonsertib) and caspase inhibitors (e.g., emricasan [pan-caspase inhibitor])-apoptosis inhibitors reduce liver steatosis and fibrosis; lysyl oxidase-like 2 (LOXL2) inhibitors, such as simtuzumab-LOXL2 is a key matrix enzyme in collagen formation and is highly expressed in the liver; galectin-3 inhibitors, such as GR-MD-02 and TD139-galectin-3 is critical for development of liver fibrosis; antioxidants, including vitamin E (e.g., a-tocopherol) and scavengers of reactive oxygen species (ROS) and free radicals (e.g., cysteamine, glutathione, melatonin and pentoxifylline [also anti-inflammatory via inhibition of TNF-a and phosphodiesterases])—vitamin E reduces liver steatosis, hepatocyte ballooning and lobular inflammation; and, analogs, derivatives and salts thereof. In some embodiments, a peptide product described herein is used in conjunction with a PPAR agonist (e.g., a PPAR-δ agonist such as elafibranor or/and a PPAR-γ agonist such as pioglitazone), a HMG-CoA reductase inhibitor (e.g., a statin such as rosuvastatin), an FXR agonist (e.g., obeticholic acid) or an antioxidant (e.g., vitamin E), or any combination thereof, to treat NAFLD (e.g., NASH). In certain embodiments, the one or more additional therapeutic agents for treatment of NAFLD (e.g., NASH) are or include vitamin E or/and pioglitazone. Other combinations may also be used as would be understood by those of ordinary skill in the art.

Pharmacokinetic (“PK”) parameters can be estimated using Phoenix® WinNonlin® version 8.1 or higher (Certara USA, Inc., Princeton, N.J.). A non-compartmental approach consistent with the extravascular route of administration can be used for parameter estimation. The individual plasma concentration-time data can be used for pharmacokinetic calculations. In addition to parameter estimates for individual animals, descriptive statistics (e.g. mean, standard deviation, coefficient of variation, median, min, max) can be determined, as appropriate. Concentration values that are below the limit of quantitation can be treated as zero for determination of descriptive statistics and pharmacokinetic analysis. Embedded concentration values that are below the limit of quantitation can be excluded from pharmacokinetic analysis. All parameters can be generated from individual dual agonist peptide (or derivatives and/or metabolites thereof) concentrations in plasma from test article-treated groups on the day of dosing (Day 1). Parameters can be estimated using nominal dose levels, unless out of specification dose formulation analysis results are obtained, in which case actual dose levels can be used. Parameters can be estimated using nominal sampling times; if bioanalytical sample collection deviations are documented, actual sampling times can be used at the affected time points. Bioanalytical data can be used as received for the pharmacokinetic analysis and can be presented in tables and figures in the units provided. Pharmacokinetic parameters can be calculated and presented in the units provided by the analytical laboratory (the order of magnitude can be adjusted appropriately for presentation in the report, e.g., h*ng/mL converted to h*μg/mL). Descriptive statistics (e.g., mean, standard deviation, coefficient of variation, median, min, max) and pharmacokinetic parameters can be determined to three significant figures, as appropriate. Additional data handling items can be documented as needed. PK parameters to be determined, as data permit, can include but are not limited to the following: Cₘₐₓ: Maximum observed concentration; DN Cₘₐₓ: dose normalized maximum concentration, calculated as Cₘₐₓ/dose; Tₘₐₓ: time of maximum observed concentration; AUC₀₋ₜ: area under the curve from time 0 to the time of the last measurable concentration, calculated using the linear trapezoidal rule; AUC₀₋₉₆: area under the curve from time 0 to hour 96, calculated using the linear trapezoidal rule; DN AUC₀₋₉₆: dose normalized AUC₀₋₉₆, calculated as AUC₀₋₉₆/dose; AUC_{0-inf}: area under the curve from time 0 to infinity (Day 1 only), calculated as AUC_{0-inf}=AUC₀₋ₜ+Cₜ/λ_{z}, where Cₜ is the last observed quantifiable concentration and λ_{z} is the elimination rate constant; t_{1/2}: elimination half-life, calculated as ln(2)/λ_{z}. Additional parameters and comparisons (e.g. sex ratios, dose proportionality ratios, etc.) can also be determined, as would be understood by those of ordinary skill in the art.

In some embodiments, this disclosure provides pharmaceutical dosage formulation(s) comprising at least one dual agonist peptide with affinity for glucagon-like peptide 1 receptor (GLP-1R) and glucagon receptor (GCGR) wherein: the peptide is modified with a hydrophobic surfactant; the dosage is configured to control blood glucose and/or induce weight loss, with reduction of one or more adverse events as compared to an agonist with unbalanced affinity for GLP-1R and GCGR, the adverse events being selected from nausea, vomiting, diarrhea, abdominal pain and constipation, upon administration to a mammal. In some embodiments, the dual agonist peptide is any one of SEQ ID NOS: 1-10 or 12-27, or a derivative thereof, or a combination thereof. In some embodiments, the dual agonist peptide has about equal affinity for GLP-1R and GCGR, and in even more preferred embodiments is SEQ ID NO: 1. In some embodiments, administration of the dual agonist peptide to a mammal, as compared to administration of an approximate equimolar dosage of semaglutide, results in: lower blood glucose at about 48 or 96 hours following administration (optionally at least about any of 10, 20, 30, 40, or 50% lower, preferably at least about 50% lower); lower blood glucose at about 72 hours following administration (optionally at least about any of 10, 20, 30, 40, 50, 60, 70, 80, 90, or 100% lower, preferably at least about 100% lower); and/or, lower blood glucose at about 120 hours following administration. In some embodiments, administration of the dual agonist peptide to a mammal, as compared to administration of an approximate equimolar dosage of semaglutide, induces whole-body weight loss; and/or, induces liver weight loss. In some embodiments, administration of the dual agonist peptide to a mammal, as compared to administration of an approximate equimolar dosage of semaglutide, exhibits a lower Cmax (optionally at least about any of 10, 20, 30, 40, 50% lower, preferably at least about 50% lower); exhibits approximately equal or greater Tₘₐₓ (optionally at least about any of 10, 20, 30, 40, 50, 60, 70, 80, 90, or 100% greater, preferably at least about 100% greater); exhibits a similar AUC_{(0-inf)} (optionally at least about any of 50, 60, 70, 80, 90, 95, 100% thereof, preferably at least about 80-90% thereof, such as about 85-93% thereof); exhibits about an equal or higher T_{1/2(hr)} (optionally at least about any of 10, 20, 30, 40, 50, 60, 70, 80, 90, or 100% thereof, preferably at least about 50 or 75% thereof, such as about 50-75% thereof); exhibits a prolonged MRT (hr) (optionally at least about any of 10, 20, 30, 40, or 50% higher, preferably at least about 25% higher); exhibits a protracted PK/PD profile; exhibits equal or greater glucoregulatory effects; induces greater whole-body weight loss, optionally about twice thereof; induces lower body fat mass, optionally about any of 10, 20, 30, 40, 50, 60, 70, 80, 90, or 100% lower, preferably at least about 100% lower); and/or, when administered to treat NASH induces increased whole-body weight reduction, liver weight loss, improved NAS score, improved hepatosteatosis, improved ballooning, improved col1A1 staining, improved ALT, improved liver TG/TC, and improved plasma TG/TC. In some embodiments, administration of the dual agonist peptide to a mammal, as compared to administration of an approximate equimolar dosage of semaglutide, results in greater loss in body weight by approximately 14 days following administration of the dosage formulation (optionally at least about 10, 20, 30, 40 or 50% greater, preferably at least about 15% greater); and/or, greater loss in body weight by approximately 20-28 days following administration of the dosage formulation (optionally at least about any of 10, 20, 30, 40, or 50% greater, preferably at least about 25% greater). In some embodiments, administration of the dual agonist peptide to a mammal, as compared to administration of an approximate equimolar dosage of semaglutide, results in weight loss in an obese mammal sufficient to return the mammal the normal weight range of a lean normal mammal.

“Reducing,” or “reduction of” adverse effects or events refers to a reduction in the degree, duration, and/or frequency of adverse effects experienced by a subject and incidence in a group of subjects following administration of an agonist with about balanced affinity to GLP1R and GCGR compared to an agonist with unbalanced affinity for GLP1R and GCGR. Such reduction encompasses the prevention of some adverse effects that a subject would otherwise experience in response to an agonist with unbalanced affinity to GLP1R and GCGR. Such reduction also encompasses the elimination of adverse effects previously experienced by a subject following administration of an agonist with unbalanced affinity to GLP1R and GCGR. In some embodiments, “reducing,” or “reduction of” adverse effects encompass a reduction of gastrointestinal side effects wherein the adverse events are reduced to zero or undetectable levels. In other embodiments, adverse effect is reduced to level equivalent to untreated subjects but not completely eliminated. Moreover, administration of analogs with unbalanced affinity toward GLP-1R or GCGR to a mammal may lead to the need for an excessively high dose to maximally activate the receptor with weaker sensitivity toward the ligand, thus leading to a potential for exceeding the biologically effective dose level for the other ligand and causing dose-related, undesired side effects.

This disclosure also provides methods for lowering and/or stabilizing the blood glucose of a mammal, the method comprising administering a pharmaceutical dosage formulation comprising a dual agonist peptide of SEQ ID NOS. 1-10 or 12-27 (or a derivative thereof), preferably a dual agonist peptide with about equal affinity for GLP-1R and GCGR (preferably SEQ ID NO: 1), to a mammal, wherein the method reduces the incidence of, or the severity of, one of more adverse events as compared to an agonist with unbalanced affinity for GLP-1R and GCGR (e.g., semaglutide), the adverse events being selected from nausea, vomiting, diarrhea, abdominal pain and constipation, upon administration to a mammal. In some embodiments, such methods, as compared to a method in which an approximate equimolar dosage of semaglutide is administered, result in lower blood glucose (10, 20, 30, 40, 50, 60, 70, 80, 90, or 100% lower, preferably at least about 50% lower) at approximately 48 or 96 hours following administration, lower blood glucose at approximately 72 hours following administration (optionally at least about any of 10, 20, 30, 40, 50, 60, 70, 80, 90, or 100% lower, preferably at least about 100% lower), and/or, lower blood glucose at approximately 120 hours following administration (optionally at least about any of 10, 20, 30, 40, 50, 60, 70, 80, 90, or 100% lower, preferably at least about 100% lower); induces whole-body weight loss and/or induces liver weight loss; a lower Cmax (optionally about 10, 20, 30, 40, 50, 60, 70, 80, 90, or 100% lower, preferably about 40-50% lower), approximately equal or greater Tₘₐₓ (optionally about 10, 20, 30, 40, 50, 60, 70, 80, 90, or 100% lower, preferably at least about 100% greater Tₘₐₓ), a similar AUC_{(0-inf)} (optionally at least about any of 50, 60, 70, 80, 90, 95, 100% thereof, preferably at least about 80-90% thereof, such as about 85-93% thereof), approximately equal or greater T_{1/2(hr)} (optionally at least about any of 10, 20, 30, 40, 50, 60, 70, 80, 90, or 100% lower, preferably at least about 50 or 75% thereof, or about 50-75% thereof); a prolonged MRT (hr) (optionally prolonged by at least about any of 10, 20, 30, 40, 50, 60, 70, 80, 90, or 100%, preferably at least about 25%); a protracted PK/PD profile; equal or greater glucoregulatory effects; greater whole-body weight loss (optionally about twice the whole-body weight loss); lower body fat mass (optionally at least about any of 10, 20, 30, 40, 50, 60, 70, 80, 90, or 100% lower, preferably at least about 100% lower); greater loss in body weight by approximately 14 days following administration of the dosage formulation (optionally at least about any of 10, 20, 30, 40, 50, 60, 70, 80, 90, or 100% greater, preferably at least about 15% greater); greater loss in body weight by approximately 20-28 days following administration of the dosage formulation (optionally at least about any of 10, 20, 30, 40, 50, 60, 70, 80, 90, or 100% greater, preferably at least about 25% greater); and/or, weight loss in an obese mammal sufficient to return the weight of the mammal to the normal weight range of a lean normal mammal; and/or, when the method is for treating NASH, increased whole-body weight reduction, liver weight loss, improved NAS score, improved hepatosteatosis, improved ballooning, improved col1A1 staining, improved ALT, improved liver TG/TC, and improved plasma triglycerides (TG)/total cholesterol (TC).

In some embodiments, this disclosure provides pharmaceutical dosage formulations comprising an agonist peptide product (preferably SEQ ID NO: 1) and about 0.025-0.075% (w/w) polysorbate 20 (PS-20, Tween 20), about 0.2-0.5% (w/w) arginine, about 3-6% (w/w) mannitol in deionized water (pH 7.7±0.1). In preferred embodiments, the pharmaceutical dosage formulation is ALT-801 comprising SEQ ID NO: 1, about 0.050% (w/w) polysorbate 20, about 0.348% (w/w) arginine, and about 4.260% (w/w) mannitol in deionized water (pH 7.7±0.1).

In some embodiments, the F58 formulation (i.e., pharmaceutical formulation comprising ALT-801 as the API) can be modified to include a higher concentration of surfactant, such as Polysorbate 20 (PS-20), to maintain micelle formation in the formulation. See Example 8. These results identify the minimum concentration of PS-20 to be used across a range of ALT-801 concentrations in order to achieve its critical micelle concentration (CMC). The concentration of PS-20 (i.e., 0.5 mg/ml) in the F58 formulation can be raised to achieve the CMC and avoid a hazy appearance (indicative of larger aggregates precipitating from solution) of the solution when stored at +2-8° C. As shown in Example 8 herein, this can be achieved by modifying the F58 formulation to include at least about 0.66 mg of PS-20 per mg of the peptide (preferably SEQ ID NO: 1) to achieve the CMC. In some embodiments, the F58 formulation can be modified to substitute PS-20 with polysorbate 80 (PS-80, Tween 80) in an amount of at least about 1.03 mg of polysorbate 80 (PS-80, Tween 80) per mg of peptide (preferably SEQ ID NO: 1) to achieve the CMC.

In some embodiments, the pharmaceutical dosage formulation comprises a preservative. In certain embodiments, the preservatives can be selected from Methyl Paraben, Ethyl Paraben, Propyl Paraben, Butyl Paraben, Benzyl Alcohol, Chlorobutanol, Phenol, Meta cresol, Chloro cresol, Benzoic acid, Sorbic acid, Thiomersal, Phenylmercuric nitrate, Bronopol, Propylene Glycol, Benzylkonium Chloride, or Benzethonium Chloride.

In some embodiments, this disclosure provides pharmaceutical dosage formulations configured for administering to the mammal the agonist peptide product (e.g., SEQ ID NO: 1) at less than about 0.72 mg/kg/dose, optionally from about 0.001 to 0.72 mg/kg/dose. In some embodiments, the pharmaceutical dosage formulation is configured to administer less than 0.36 mg/kg/dose of the agonist peptide product to the mammal. In some embodiments, the methods comprise administering between 0.001-0.3 mg/kg/dose, optionally about 0.007 mg/kg, or about 0.014 mg/kg or about 0.03 mg/kg, or about 0.07 mg/kg, or about 0.18 mg/kg/dose or about 0.25 mg/kg/dose. In some embodiments, the pharmaceutical dosage formulation can be configured to administer between about 0.05 to about 20 mg per week; optionally 0.1 to about 10 mg per week or optionally about 1 to about 7 mg per week; or optionally about 1 to 5 mg per week. In some embodiments, the pharmaceutical dosage formulation is configured to be administered to the mammal once weekly for up to six weeks. In some embodiments, this disclosure provides pharmaceutical dosage formulations configured such that the time to reach a therapeutic dose is about four weeks or less. In some embodiments, the therapeutic dose exhibits a Cₘₐₓ of from about 10 to about 2000 ng/ml; a Tₘₐₓ of from about 10 to about 168 hours; and/or, an AUC₀₋₁₆₈ of from about 1,000 to 100,000 h*ng/mL. In some embodiments, ALT-801 may be repeatedly administered to achieve a plasma a concentration of about 5 to 1000 ng/ml or about 50 ng/ml, or about 150 ng/ml, or about 250 ng/ml or about 500 ng/ml.

In some embodiments, this disclosure provides the methods described herein that comprise administering to the mammal the agonist peptide product at less than about 0.72 mg/kg/dose, optionally from about 0.001 mg/kg/dose to less than about 0.36 mg/kg/dose, or optionally about 0.36 mg/kg/dose. In preferred embodiments of such methods, less than about 0.36 mg/kg/dose is administered to the mammal. In some embodiments, each dose is administered about once per week or once every two weeks, optionally for at least one month; optionally wherein each dose comprises about the same about of agonist peptide product. In some embodiments, such methods comprise administering about 0.72 mg/kg/dose once followed by one or more subsequent doses of from about 0.001 mg/kg/dose to about 0.36 mg/kg/dose. In some embodiments, the methods comprise administering between 0.001-0.30 mg/kg/dose, optionally about 0.007 mg/kg, or about 0.014 mg/kg or about 0.03 mg/kg, or about 0.07 mg/kg, or about 0.18 mg/kg/dose or about 0.25 mg/kg/dose. In some embodiments, the pharmaceutical dosage formulation can be configured to administer between about 0.05 to about 20 mg per week; optionally 0.1 to about 10 mg per week or optionally about 1 to about 7 mg per week; or optionally about 1 to 5 mg per week.

In preferred embodiments, such methods comprise administering the pharmaceutical dosage formulation subcutaneously. In some embodiments, such methods comprising administering the pharmaceutical dosage formulation to a mammal at about 0.03 to 0.25 mg/kg/dose exhibits a Cₘₐₓ of from about 50 to about 1000 ng/ml; a Tₘₐₓ of from about 10 to about 96 hours; and/or, an AUC₀₋₁₆₈ of from about 5,000 to 80,000 h*ng/mL. In some such methods, the time to reach a therapeutic dose is about four weeks or less. In some embodiments, the therapeutic dose exhibits a Cₘₐₓ of from about 50 to about 700 ng/ml; a Tₘₐₓ of from about 10 to about 72 hours; and/or, an AUC₀₋₁₆₈ of from about 6,000 to 70,000 h*ng/mL.

In some embodiments, the methods disclosed herein do not comprise a treatment initiation phase. In other words, the first administered dose is therapeutic without the need to titrate to avoid adverse gastrointestinal side effects. For instance, in some embodiments, the method can comprise administering a first one or more doses (the treatment initiation phase) of a peptide of this disclosure, such as SEQ ID NO: 1, followed by subsequent second one or more and higher doses of the peptide, each of the first and second doses being administered for one or more weeks. In some embodiments, the first dose(s) and the second dose(s) can be followed by one or more third doses that can be higher than the second dose(s). The switch from the first dose, the second dose, and the third dose can be made on a weekly basis. For instance, if it appears the first dose has not induced lower blood glucose and/or weight loss after one or more weeks, the second higher dose can then be administered for one or more weeks followed by an analysis of the effects of the second dose(s). If the beneficial effects are observed (e.g., lower blood glucose and/or body weight), the second dose can continue to be administered. If the beneficial effects are not observed, the third dose may be administered for one or more weeks, followed by a determination of beneficial effects. This cycle of dosing and analysis can be repeated as appropriate, provided adverse events are not observed with each dose. In some embodiments, the subsequent second one or more and higher doses of the peptide can be administered because glycemic control (e.g., decreased blood glucose) was not achieved after about four weeks of administration of the first one or more doses. In some embodiments, the first one or more doses can be administered without the intention to produce a therapeutic effect (e.g., decreased blood glucose and/or weight loss). In some embodiments, however, the methods can be carried out without including the treatment initiation phase.

In some embodiments, the methods can be a first line indication for blood glucose control and/or weight loss in a human being, meaning that it is the first and sole active agent administered to the patient for the purpose of controlling blood glucose and/or inducing weight loss in the human being. In some embodiments, the methods disclosed herein can include an adjunct treatment of diet and/or exercise. In such embodiments, the human being can be administered the pharmaceutical dosage and provided with instructions regarding diet and/or exercise that can enhance the beneficial effects of the pharmaceutical dosage. In some embodiments, the human being to whom the pharmaceutical dosage is administered has type 2 diabetes mellitus. In some embodiments, the human being can exhibit established cardiovascular disease, with or without type 2 diabetes mellitus.

In some embodiments, the pharmaceutical dosage is administered about weekly. In some embodiments, the pharmaceutical dosage is administered to the human being about weekly from about 2 weeks to about 8 weeks, or longer. In some embodiments, the pharmaceutical dosage administered to the human being as a weekly dose for about 4 to about 8 weeks, optionally about 6 weeks, as compared to administration of an approximate equimolar dosage of semaglutide results in greater whole-body weight loss at about 1 week, about 2 weeks, about 3 weeks, about 4 weeks, about 5 weeks, about 6 weeks, or about 7 weeks following administration to the human being. In some embodiments, the pharmaceutical dosage is administered on about days 1, 8, 15, 22, 29, and 36. In some embodiments, the methods can include administration to the human being of a single dose, as compared to administration of an approximate equimolar dosage of semaglutide, results in lower blood glucose at about 1 day, about 2 days, about 3 days, about 4 days, about 5 days, about 6 days or about 7 days following administration. In some embodiments, the methods can include administration to human being of a weekly dose for about 4 to about 8 weeks, optionally about 6 weeks, as compared to administration of an approximate equimolar dosage of semaglutide, results in greater whole-body weight loss at about 1 week, about 2 weeks, about 3 weeks, about 4 weeks, about 5 weeks, about 6 weeks or about 7 weeks following administration. In some embodiments, the methods can include administration to the human being of a single dose, as compared to administration of an approximate equimolar dosage of semaglutide, exhibits a lower Cₘₐₓ at about 1 day, about 2 days, about 3 days, about 4 days, about 5 days, about 6 days or about 7 days following administration. In some embodiments, the methods can include administering the pharmaceutical dosage to an adult human at from about 0.5 mg/dose, about 1.0 mg/dose, about 1.5 mg/dose, about 2.0 mg/dose, about 2.5 mg/dose, about 3.0 mg/dose, about 3.5 mg/dose, about 4.0 mg/dose, about 4.5 mg/dose, about 5.0 mg/dose, or about 5.5 mg/dose. In some embodiments, the pharmaceutical dosage can be administered about once per week or once every two weeks, optionally for at least one month; optionally wherein each dose comprises about the same amount of agonist peptide product. In some embodiments, the pharmaceutical dosage can be administered subcutaneously. In some embodiments, one or more of the doses can be administered via a first route (e.g., subcutaneously) and subsequently administered by a different route (e.g., orally). In some embodiments, the time to reach a therapeutic dose is about four weeks or less. In some embodiments, administration of the pharmaceutical dosage formulation exhibits a Cₘₐₓ of from about 400 to about 1300 ng/ml; a Tₘₐₓ of from about 10 to about 36 hours; and/or, an AUC₀₋₄₈ of from about 15,000 to 45,000 h*ng/mL. In preferred embodiments, the weight loss in the human being is at least 5%, at least 10%; or from about 1% to about 20%; or from about 5% to about 10% (w/w). In some embodiments, administration thereof to a mammal results weight loss in an obese mammal sufficient to return the human being the normal weight range of a lean normal human being. In some embodiments, administration to a human being with a body mass index (BMI) indicative of obesity (e.g., about 30 or higher) exhibit a decrease in body weight of about 5-20%, such as about 15%, for an appropriate time (e.g., after any of about two, four, eight, 10, 20, or 30-100 weeks, such as about any of 50, 60, or 70 weeks). In preferred embodiments, the weight loss in such human beings is significant (e.g., P<0.001, 95% confidence interval (CI)). In some preferred embodiments, within about four weeks, administration to a human being results in at least about a 2-5% reduction in body weight, and in some embodiments continues and/or stabilizes until administration ceases. In some embodiments, in addition to weight loss, administration can also improve cardiovascular risk factors including greater reductions in waist circumference, BMI, systolic and diastolic blood pressures, HbA1c, fasting plasma glucose, C-reactive protein, and/or fasting lipid levels, as well as in some embodiments physical functioning scores and quality of life. In some embodiments, the pharmaceutical dosage form is an aqueous formulation comprising one or more of polysorbate 20, Arginine, or Mannitol.

### Specific Aspects of the Disclosure

Preferred aspects of this disclosure include:

A pharmaceutical dosage formulation comprising an agonist peptide product with affinity for glucagon-like peptide 1 receptor (GLP-1R) and glucagon receptor (GCGR) wherein: the peptide is modified with a non-ionic glycolipid surfactant; the dosage is configured to improve control of blood glucose with reduction of one or more adverse events as compared to an agonist with unbalanced affinity for GLP-1R and GCGR, the adverse events being selected from nausea, vomiting, diarrhea, abdominal pain and constipation, upon administration to a mammal.

A pharmaceutical dosage formulation comprising an agonist peptide with affinity for glucagon-like peptide 1 receptor (GLP-1R) and glucagon receptor (GCGR) wherein: the peptide is modified with a non-ionic glycolipid surfactant; the dosage is configured to induce weight loss with reduction of one or more adverse events as compared to an agonist with unbalanced affinity for GLP-1R and GCGR, the adverse events being selected from nausea, vomiting, diarrhea, abdominal pain and constipation, upon administration to a mammal.

A pharmaceutical dosage formulation of any preceding aspect, wherein weight loss is at least 5%, at least 10%; or from about 1% to about 20%; or from about 5% to about 10% (w/w).

A pharmaceutical dosage formulation of any preceding aspect, wherein the dosage is configured as a weekly dosage form, optionally configured for administration from about 2 weeks to about 8 weeks.

The pharmaceutical dosage formulation of the preceding aspect, wherein administration to a mammal of a single dose, as compared to administration of an approximate equimolar dosage of semaglutide, results in lower blood glucose at about 1 day, about 2 days, about 3 days, about 4 days, about 5 days, about 6 days or about 7 days following administration.

The pharmaceutical dosage formulation of the preceding aspect, wherein administration to a mammal of a weekly dose for about 4 to about 8 weeks, optionally about 6 weeks, as compared to administration of an approximate equimolar dosage of semaglutide, results in greater whole-body weight loss at about 1 week, about 2 weeks, about 3 weeks, about 4 weeks, about 5 weeks, about 6 weeks or about 7 weeks following administration.

The pharmaceutical dosage formulation of the preceding aspect, wherein administration to a mammal of a single dose, as compared to administration of an approximate equimolar dosage of semaglutide, exhibits a lower Cₘₐₓ at about 1 day, about 2 days, about 3 days, about 4 days, about 5 days, about 6 days or about 7 days following administration.

The pharmaceutical dosage formulation of any preceding aspect wherein the dual agonist peptide is any one of SEQ ID NOS: 1-10 or 12-27.

The pharmaceutical dosage formulation of any preceding aspect, wherein the dual agonist peptide has about equal affinity for GLP-1R and GCGR, optionally wherein said dual agonist peptide is SEQ ID NO: 1.

The pharmaceutical dosage formulation of any preceding aspect, wherein the surfactant is a 1-alkyl glycoside class surfactant.

The pharmaceutical dosage formulation of any preceding aspect present as an aqueous formulation comprising one or more of polysorbate 20, Arginine, or Mannitol.

The pharmaceutical dosage formulation of any preceding aspect wherein administration thereof to a mammal, as compared to administration of an approximate equimolar dosage of semaglutide, results in:
lower blood glucose at about 48 or 96 hours following administration, optionally wherein it is about 50% lower;
lower blood glucose at about 72 hours following administration, optionally wherein it is about 100% lower; and/or,
lower blood glucose at about 120 hours following administration.

The pharmaceutical dosage formulation of any preceding aspect wherein:
a) administration of the dosage formulation to a mammal:
   induces whole-body weight loss; and/or,
   induces liver weight loss;
and/or,
b) administration of the dosage formulation to a mammal, as compared to semaglutide administered at an approximately equimolar dose:
   exhibits a lower Cmax, optionally about 50% lower;
   exhibits approximately equal or greater Tmax, optionally about 100% longer;
   exhibits a similar AUC_{(0-inf)}, optionally about 85-93% of thereof;
   exhibits an approximately equal or longer T1/2 (hr), optionally about 25-75% thereof;
   exhibits a prolonged MRT (hr), optionally at least about 25% higher;
   exhibits a protracted PK/PD profile;
   exhibits about equal or greater glucoregulatory effects;
   induces greater whole-body weight loss, optionally about twice thereof;
   induces lower body fat mass, optionally about 50 to 100% lower; and/or,
   when administered to treat NASH induces increased whole-body weight reduction, liver weight loss, improved NAS score, improved hepatosteatosis, improved ballooning, improved col1A1 staining, improved ALT, improved liver TG/TC, and improved plasma TG/TC.

The pharmaceutical dosage formulation of the preceding aspect, wherein administration to a mammal, as compared to semaglutide administered at an approximately equimolar dose: results in greater loss in body weight by approximately 14 days following administration of the dosage formulation, optionally about 15% greater; and/or, results in greater loss in body weight by approximately 20-28 days following administration of the dosage formulation, optionally about 25% greater.

The pharmaceutical dosage formulation of any preceding aspect wherein administration thereof to a mammal results weight loss in an obese mammal sufficient to return the mammal the normal weight range of a lean normal mammal.

The pharmaceutical dosage formulation according to any preceding aspect, wherein the pharmaceutical dosage formulation comprises one or more pharmaceutically acceptable excipients selected from a buffer, or an osmolarity adjuster.

The pharmaceutical dosage formulation according to any preceding aspect, wherein the pharmaceutical dosage formulation further comprises a surfactant.

The pharmaceutical dosage formulation according to any preceding aspect, wherein the concentration of the dual peptide agonist is 0.05 to 20 mg/ml.

The pharmaceutical dosage formulation according to any preceding aspect, wherein the concentration of the dual peptide agonist is 0.1 to 10 mg/ml.

The pharmaceutical dosage formulation according to any preceding aspect, wherein the pH of the dual peptide agonist is between 6 to 10.

The pharmaceutical dosage formulation according to any preceding aspect, the formulation comprising about 0.025-0.15% (w/w) polysorbate 20 or polysorbate 80, about 0.2-0.5% (w/w) arginine, about 3-6% (w/w) mannitol in water (pH 7.7±1.0); optionally about 0.050% (w/w) polysorbate 20, about 0.35% (w/w) arginine, about 4.3% (w/w) mannitol in water (pH 7.7±1.0).

The pharmaceutical dosage formulation according to any preceding aspect, wherein the formulation comprising, about 0.2-0.5% (w/w) arginine, about 3-6% (w/w) mannitol and 0.6 to 1.0 mg of polysorbate 20 or 1.0 to 1.5 mg of polysorbate 80 per mg of ALT-801 (SEQ ID NO: 1) in water (pH 7.7±1.0) in water (pH 7.7±1.0).

The pharmaceutical dosage formulation of any preceding aspect configured to be administered to the mammal wherein the agonist peptide product is at less than about 0.25 mg/kg/dose, optionally greater than about 0.001 mg/kg/dose to less than about 0.15 mg/kg/dose.

The pharmaceutical dosage formulation of the preceding aspect configured to administer less than 0.25 mg/kg/dose of the agonist peptide product to the mammal.

The pharmaceutical dosage formulation of the preceding aspect configured to administer between 0.001-0.15 mg/kg/dose, optionally about 0.03 mg/kg/dose or about 0.10 mg/kg/dose.

The pharmaceutical dosage formulation of any preceding aspect wherein configured to administer to a human between about 0.1 to about 15 mg per week; optionally about 1 to about 7 mg per week; or optionally about 1 to 5 mg per week.

The pharmaceutical dosage formulation of any preceding aspect configured to be administered to the mammal once weekly for at least, or up to six weeks.

The pharmaceutical dosage formulation of any preceding aspect configured such that the time to reach a therapeutic dose is about four weeks or less.

The pharmaceutical dosage formulation of the preceding aspect wherein the therapeutic dose exhibits a Cₘₐₓ of from about 10 to about 300 ng/ml; a Tₘₐₓ of from about 10 to about 36 hours; and/or, an AUC₀₋₁₆₈ of from about 1,000 to 100,000 h*ng/mL.

A method for lowering the blood glucose of a mammal, the method comprising administering pharmaceutical dosage formulation of any preceding claim to a mammal, wherein the method:
a) reduces the incidence of one of more adverse events as compared to an agonist with unbalanced affinity for GLP-1R and GCGR, the adverse events being selected from nausea, vomiting, diarrhea, abdominal pain and constipation, upon administration to a mammal;
b) as compared to a method in which an approximate equimolar dosage of semaglutide is administered, results in: approximately 50% lower blood glucose at approximately 48 or 96 hours following administration, approximately 100% lower blood glucose at approximately 72 hours following administration, and/or, lower blood glucose at approximately 120 hours following administration;
c) induces whole-body weight loss and/or induces liver weight loss;
d) as compared to a method in which an approximate equimolar dosage of semaglutide is administered, results in:
   a lower Cmax or optionally about 50% lower Cmax;
   approximately equal or greater Tmax or optionally about 100% greater Tmax,
   a similar AUC(0-inf) or optionally approximately 85-93% AUC_{(0-inf)};
   approximately equal or lesser T1/2 (hr) or optionally approximately 50-75% T_{1/2} (hr);
   a prolonged MRT (hr) or optionally at least approximately 25% higher MRT (hr);
   a protracted PK/PD profile, exhibits equal or greater glucoregulatory effects;
   greater whole-body weight loss or optionally approximately twice the whole-body weight loss;
   lower body fat mass, optionally about 100% lower the body fat mass; and/or,
   increased whole-body weight reduction, liver weight loss, improved NAS score, improved hepatosteatosis, improved ballooning, improved col1A1 staining, improved ALT, improved liver TG/TC, and improved plasma TG/TC, when the method is for treating NASH;
e) as compared to semaglutide administered at an approximately equimolar dose: results in greater loss in body weight by approximately 14 days following administration of the dosage formulation, optionally about 15% greater; and/or, results in greater loss in body weight by approximately 20-28 days following administration of the dosage formulation, optionally about 25% greater; and/or,
f) weight loss in an obese mammal sufficient to return the weight of the mammal to the normal weight range of a lean normal mammal.

A method for inducing weight loss in a mammal, the method comprising administering pharmaceutical dosage formulation of any preceding claim to a mammal, wherein the method reduces the incidence of one of more adverse events as compared to an agonist with unbalanced affinity for GLP-1R and GCGR, the adverse events being selected from nausea, vomiting, diarrhea, abdominal pain and constipation, upon administration to a mammal.

The method of the preceding aspects wherein the dual agonist peptide is any one of SEQ ID NOS: 1-10 or 12-27.

The method of the preceding aspects, wherein the dual agonist peptide has about equal affinity for GLP-1R and GCGR, optionally wherein said dual agonist peptide is SEQ ID NO: 1.

The method of the preceding aspects, wherein the pharmaceutical dosage is administered about weekly.

The method of any preceding aspect, wherein the pharmaceutical dosage is administered is administered subcutaneously.

The method of any preceding aspect, wherein the pharmaceutical dosage is administered about weekly from about 2 weeks to about 8 weeks, or longer.

The method of any preceding aspect, wherein administering the pharmaceutical dosage to the mammal as a weekly dose for about 4 to about 8 weeks, optionally about 6 weeks, as compared to administration of an approximate equimolar dosage of semaglutide results in greater whole-body weight loss at about 1 week, about 2 weeks, about 3 weeks, about 4 weeks, about 5 weeks, about 6 weeks or about 7 weeks following administration to the mammal.

The method of any preceding aspect comprising administering to the mammal the agonist peptide product at less than about 0.25 mg/kg/dose, optionally greater than about 0.001 mg/kg/dose to less than about 0.15 mg/kg/dose.

The method of the preceding aspect wherein the mammal is administered less than about 0.25 mg/kg/dose.

The method of any preceding aspect configured to administer the agonist peptide product at between 0.001-0.15 mg/kg/dose, optionally about 0.03 mg/kg/dose or about 0.10 mg/kg/dose.

The method of any preceding aspect wherein each dose is administered about once per week or once every two weeks, optionally for at least one month; optionally wherein each dose comprises about the same about of agonist peptide product.

The method of any preceding aspect comprising administering about less than 0.25 mg/kg/dose once followed by one or more subsequent doses of from about 0.03 mg/kg/dose to about 0.10 mg/kg/dose.

The method of any preceding aspect comprising administering the agonist peptide product at between 0.001-0.15 mg/kg/dose.

The method of any preceding aspect wherein the pharmaceutical dosage formulation comprises about 0.025-0.15% (w/w) polysorbate 20 or polysorbate 80, about 0.2-0.5% (w/w) arginine, about 3-6% (w/w) mannitol in water (pH 7.7±1.0); optionally about 0.050% (w/w) polysorbate 20, about 0.35% (w/w) arginine, about 4.3% (w/w) mannitol in water (pH 7.7±1.0); optionally wherein the dual agonist peptide is SEQ ID NO: 1.

The method of any preceding aspect, wherein the formulation comprises about 0.2-0.5% (w/w) arginine, about 3-6% (w/w) mannitol and 0.6 to 1.0 mg of polysorbate 20 or 1.0 to 1.5 mg of polysorbate 80 per mg of ALT-801 (SEQ ID NO: 1) in water (pH 7.7±1.0) in water (pH 7.7±1.0).

The method of any preceding aspect wherein administering the pharmaceutical dosage formulation is configured to administer to a human between about 0.1 to about 15 mg per week; optionally about 1 to about 7 mg per week; or optionally about 1 to 5 mg per week.

The method of any preceding aspect wherein time to reach a therapeutic dose is about four weeks or less.

A pharmaceutical dosage formulation configured for subcutaneous administration comprising an agonist peptide product with affinity for glucagon-like peptide 1 receptor (GLP-1R) and glucagon receptor (GCGR) wherein the peptide product is represented as SEQ ID NO: 1; the dosage is configured to improve control of blood glucose with reduction of one or more adverse events as compared to an agonist with unbalanced affinity for GLP-1R and GCGR, the adverse events being selected from nausea, vomiting, diarrhea, abdominal pain and constipation, upon administration to a mammal.

A pharmaceutical dosage formulation configured for subcutaneous administration comprising an agonist peptide with affinity for glucagon-like peptide 1 receptor (GLP-1R) and glucagon receptor (GCGR) wherein the peptide product is represented as SEQ ID NO: 1; the dosage is configured to induce weight loss with reduction of one or more adverse events as compared to an agonist with unbalanced affinity for GLP-1R and GCGR, the adverse events being selected from nausea, vomiting, diarrhea, abdominal pain and constipation, upon administration to a mammal.

The pharmaceutical dosage formulation of the preceding aspect, wherein weight loss is at least 5%, at least 10%; or from about 1% to about 20%; or from about 5% to about 10% (w/w).

The pharmaceutical dosage formulation of any preceding aspect, wherein the dosage is configured as a weekly dosage form, optionally configured for administration from about 2 weeks to about 8 weeks.

The pharmaceutical dosage formulation according to any preceding aspect, wherein the formulation comprises about 0.2-0.5% (w/w) arginine, about 3-6% (w/w) mannitol and 0.6 to 1.0 mg of polysorbate 20 or 1.0 to 1.5 mg of polysorbate 80 per mg of ALT-801 (SEQ ID NO: 1) in water (pH 7.7±1.0) in water (pH 7.7±1.0).

The pharmaceutical dosage formulation of the preceding aspect, wherein administration to a mammal of a single dose, as compared to administration of an approximate equimolar dosage of semaglutide, exhibits a lower Cₘₐₓ at about 1 day, about 2 days, about 3 days, about 4 days, about 5 days, about 6 days or about 7 days following administration.

The pharmaceutical dosage formulation of any preceding aspect, wherein the dosage is configured to administer to a human between about 0.1 to about 15 mg per week; optionally about 1 to about 7 mg per week; or optionally about 1 to 5 mg per week.

The pharmaceutical dosage formulation of any preceding aspect configured to be administered to the mammal once weekly for at least, or up to six weeks.

The pharmaceutical dosage formulation of any preceding aspect, wherein the dosage is configured to reach a therapeutic dose in about four weeks or less following first weekly administration.

The pharmaceutical dosage formulation of the preceding aspect, wherein the therapeutic dose exhibits a Cₘₐₓ of from about 10 to about 300 ng/ml, optionally a Cₘₐₓ less than 200 ng/ml; a Tₘₐₓ of from about 10 to about 36 hours; and/or, an AUC₀₋₁₆₈ of from about 1,000 to 100,000 h*ng/mL.

A method for inducing weight loss in a mammal, the method comprising administering pharmaceutical dosage formulation of any one of claims 48-57 to a mammal, wherein the method reduces the incidence of one of more adverse events as compared to an agonist with unbalanced affinity for GLP-1R and GCGR, the adverse events being selected from nausea, vomiting, diarrhea, abdominal pain and constipation, upon administration to a mammal at a therapeutic dose.

The method of the preceding aspect, wherein the pharmaceutical dosage is administered about weekly wherein an initial dose is the therapeutic dose.

The method of the preceding aspects, wherein the pharmaceutical dosage is administered about weekly from about 2 weeks to about 8 weeks, or longer.

Other aspects of this disclosure are also contemplated as will be understood by those of ordinary skill in the art.

Unless defined otherwise or clearly indicated otherwise by their use herein, all technical and scientific terms used herein have the same meaning as commonly understood by those of ordinary skill in the art to which this application belongs. As used in the specification and the appended claims, the word “a” or “an” means one or more. As used herein, the word “another” means a second or more. The acronym “aka” means also known as. The term “exemplary” as used herein means “serving as an example, instance or illustration”. Any embodiment or feature characterized herein as “exemplary” is not necessarily to be construed as preferred or advantageous over other embodiments or features. In some embodiments, the term “about” or “approximately” means within ±10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Optional or optionally means that the subsequently described event or circumstance can or cannot occur, and that the description includes instances where the event or circumstance occurs and instances where it does not. All publications, patents, and patent applications mentioned in this specification are herein incorporated by reference in their entireties to the same extent as if each individual publication, patent, or patent application was specifically and individually indicated to be incorporated by reference.

Certain embodiments are further described in the following examples. These embodiments are provided as examples only and are not intended to limit the scope of the claims in any way.

### Examples

### Example 1. Peptide Synthesis

There are many standard protecting groups and coupling agents that can be successfully used for typical N-alpha-Fmoc based peptide synthesis. Typical examples are listed in U.S. Pat. No. 9,856,306 B2, which is incorporated by reference in its entirety into this disclosure. Further examples can be found in many reviews and protocols, for example those published and routinely updated online by Novabiochem and more specialist reviews (for example Behrendt, R., et al. (2015) J Peptide Sci 22: 4-27 and references therein). Typical commercial protocols used by many contract peptide synthesis houses were used for the synthesis herein. More specialized protocols are given below.

Preparation of C-Terminal Amide Analogs—SEQ. ID. NO. 1.

A sample of Boc-His(Trt)-Aib-Gln(Trt)-Gly-Thr(tBu)-Phe-Thr(tBu)-Ser(tBu)-Asp(tBu)-Tyr(tBu)-Ser(tBu)-Lys(Boc)-Tyr(tBu)-Leu-Asp(tBu)-Glu*-Lys(ivDDE)-Ala-Ala-Lys*-Glu(tBu) Phe-Ile-Gln(Trt)-Trp(Boc)-Leu-Leu-Gln(Trt)-Thr(tBu)-Rink amide resin (SEQ ID NO:1) was prepared by sequential addition of N-alpha-Fmoc protected amino acids using standard coupling protocols, e.g. diisopropylcarbodiimide (DIC)/hydroxybenztriazole (HBT) couplings, followed by standard deprotection with piperazine, next step coupling, etc. (Glu* and Lys* indicate a side chain cyclic lactam linkage, achieved through deprotection of the allyl-based side chain protection using Pd(PPh₃)₄/1,3-dimethylbarbituric acid catalysis, washing with DIPEA in NMP and with 0.5% sodium diethyldithiocarbamate trihydrate and DIPEA, then coupling with DIC/Oxyma). Deprotection of the ivDDE group on Lys-N-epsilon position at residue 17 by incubation with 2% or more hydrazine hydrate in DMF, followed by washing by DMF/CH₂Cl₂, the Lys side chain was acylated with tert-butyl 18-([beta-D-glucuron-1-yl]oxy)octadecanoate in DMF/CH₂Cl₂ through the use of DIC/HBt or other coupling agents. Completion of the coupling was checked by ninhydrin and the product was washed extensively with CH₂Cl₂.

The product resin is submitted to final deprotection and cleavage from the resin by treatment with the cleavage cocktail (94% TFA: 2% EDT; 2% H₂O; 2% TIS) for a period of 240 min at room temperature. The mixture was treated with Et₂O, to precipitate the product and washed extensively with Et₂O to yield the crude title peptide product after drying in vacuo.

Purification is carried out in batches by reversed phase (C18) hplc. The crude peptide was loaded on a 4.1×25 cm hplc column at a flow rate of about 15 mL/min (CH₃CN organic modifier in aqueous trifluoracetic acid 0.1%, buffer A; CH3CN with 0.1% TFA, Buffer B) and eluted with a gradient from 40-70% buffer B. The product fraction is lyophilized to yield the title product peptide (SEQ. ID NO: 1) with a purity >94% by analytical hplc (10.5 min; 40-70% CH₃CN in 0.1% TFA)/mass spectrometry (M+1 peak=1937.44; molecular weight found 3872.88). In a similar manner, using the glucuronic or melibiouronic acids prepared as indicated in the examples, were prepared the other analogs of the invention.

Analytical data is shown in Table A:

TABLE A

| SEQ ID NO: | Expected MW | Found (M*2) | k′ value; hplc gradient | Column |
|---|---|---|---|---|
| 1. | 3873.34 | 3872.94 | 3.0; 40-70% B in 20 min | Luna C-18 5μ |
| 2. | 3977.47 | 3977.67 | 3.8; 45-75% B in 20 min | Luna C-18 5μ |
| 3. | 3845.28 | 3845.16 | 3.1; 40-70% B in 20 min | Luna C-18 5μ |
| 4. | 3873.34 | 3873.46 | 6.5; 40-70% B in 20 min | PLRP-S 8μ |

Compounds are analysed by hplc/MS to provide purity data and identity data (molecular ion detection). The hplc technique utilizes analytical columns packed with the materials listed, of particle size listed and the data is reported here as k′ values (k′=(Tr−T₀)/T₀) which are expected to be largely independent of system configuration and dead volume, but dependent on gradient and packing material. All compounds were reported to be circa 95% pure.

The corresponding 1-methyl and 1-octyl analogs of the title compound are prepared in a similar manner, but using the reagents 1′-methyl β-D-glucuronic acid and 1′-octyl β-D-glucuronic acid (Carbosynth). The corresponding 1-decyl, 1-dodecyl, 1-tetradecyl, 1-hexadecyl, 1-octadecyl and 1-eicosyl and higher analogs are prepared using the corresponding monosaccharide and disaccharide uronic acids, prepared as described above. Alternatively, the 1-alkyl glucuronyl, or other uronic acylated analogs, may be prepared by initial purification of the deprotected or partially deprotected peptide followed by acylation by the desired uronic acid reagent. Alternatively, the 1-alkyl glucuronyl, or other uronic acylated analogs, may be prepared by initial purification of the recombinantly prepared peptide followed by selective acylation of the side chain amino function by the desired uronic acid reagent.

### A. 1-Alkyl β-d-glucuronic Acids. General Oxidation Method

To a solution of 1-dodecyl β-d-glucopyranoside [2.0 g, 5.74 mmol] in 20 mL of acetonitrile and 20 mL of deionized water is added (diacetoxyiodo)benzene [4.4 g, 13.7 mmol] and TEMPO [0.18 g, 1.15 mmol]. The resulting mixture was stirred at room temperature until reaction completion (by 20 h). The reaction mixture was diluted with water and lyophilized to dryness to give crude product as a white powder of sufficient purity for direct use in coupling to the peptide Lys side chain (1.52 g, 73%). In a like manner were prepared the other 1-alkyl β-d-glucuronic or melibiouronic acids used to acylate the other peptide products described herein. The corresponding 1-substituted glucosides or melibosides were prepared using the procedures in these examples but substituting the appropriate chain length dicarboxylic starting materials to yield the desired chain length from the synthetic procedures of the examples, for example hexadecanedioic acid, dodecanedioic acid and the like in place of octadecanedioic acid.

### B. 18-(tertbutoxy)-18-oxooctadecanoic acid

A suspension of octadecanedioic acid (40 g, 127 mmol) in toluene (500 ml) was heated at 95° C. under nitrogen. To the resulting solution, was added N,N-dimethylformamide di-tert-butylacetal (98 g, 434 mmol), dropwise over 3-4 hr. The reaction was stirred overnight at the same temperature, concentrated to dryness in vacuo and placed under high vacuum overnight. The resulting solid was suspended in CH2Cl2 (200 ml) with heat and sonication, and filtered at RT, washing with CH2Cl2. The filtrate (2) was concentrated to give the product as a solid (45 g, 86%) which was used without further purification.

### C. Tert-butyl 18-hydroxyoctadecanoate

A solution of 18-(tertbutoxy)-18-oxooctadecanoic acid (45 g, 121 mmol) in THF was cooled over an ice bath, under nitrogen and treated dropwise with borane dimethylsulfide complex (16 ml, 158 mmol). Vigorous gas evolution occurred over the first few milliliters of addition. After the addition, the mixture was slowly allowed to warm to RT and was stirred overnight. The reaction was chilled over an ice bath, quenched with saturated sodium carbonate solution, diluted with ethyl acetate and washed with saturated sodium carbonate solution. The organic layer was concentrated in vacuo and placed under high vacuum overnight. The residue was dissolved in warm toluene (200 ml) and let stand for several hours at room temperature. The precipitated diol was removed by filtration through Celite, cake washed with toluene. The toluene solution was applied directly to a silica gel column and eluted with 10% ethyl acetate/hexane then 20% ethyl acetate/hexane, then 30% ethyl acetate/hexane and concentrated to give the product (24 g, 51%) as an oil which solidifies on standing. ¹E1 NMR (500 MHz, d₄-MeOH): δ=3.64 (m, 2H), 2.21 (t, 2H, J=9), 1.44 (s, 9H) 1.50-1.62 (m, 4H), 1.20-1.40 (m, 27H)

### D. Tert-butyl 18-([1-beta-D-glucos-1-yl]oxy)octadecanoate

Tert-butyl 18-hydroxyoctadecanoate (46 g, 129 mmol) was dissolved in toluene (400 ml), concentrated in vacuo to circa 250 ml, and allowed to come to room temperature under nitrogen. To this solution was added HgO (yellow) (22.3 g, 103 mmol), HgBr₂ (37 g, 103 mmol), and acetobrom glucose with vigorous stirring. Stirring was continued overnight until alcohol was consumed and the mixture was filtered through Celite. The filtrate was treated with copper(II)triflate (1 g) and stirred for 1 hour until the orthoester (spot above product on TLC) was degraded. The reaction was then washed with water and the organic layer was concentrated in vacuo. The residue was dissolved in methanol (500 ml) and treated with sodium methoxide (5.4 M in methanol) in 0.5 ml portions to bring the pH to 9 (spotting directly onto pH paper). The pH was checked every 0.5 hour and more sodium methoxide was added as necessary to maintain the pH at 9. The reaction was complete in 4 hr. Acetic acid was added dropwise to bring the pH to 7, and the mixture was concentrated in vacuo. The residue was loaded onto silica gel and purified by silica gel chromatography eluting with 5% methanol/CH2Cl2 then 10% methanol/CH2C12 to yield the product as a white solid (55 g, 82%). ¹HNMR (400 MHz, d₄-MeOH): δ=4.30 (d, 1H, J=7.6), 3.84 (m, 1H), 3.77 (d, 1H, J=9.6), 3.45-3.60 (m, 2H), 3.36 (t, 1H, J=9.2), 3.21 (t, 1H, J=8.4), 2.20 (t, 2H, J=7.2), 1.50-1.67 (m, 4H), 1.43 (s, 9H), 1.43-1.33 (m, 2H), 1.28 (br s, 24H)

### E. Tert-butyl 18-([beta-D-glucuron-1-yl]oxy)octadecanoate

Tert-butyl 18-([1-beta-D-glucos-1-yl]oxy)octadecanoate (50 g, 96 mmol) was dissolved in dioxane (800 ml) in a 2000 ml 3-neck flask with mechanical stirring and cooled to 10° C. To the solution was added 2,2,6,6-tetramethyl-1-piperidinyloxy (TEMPO) (150 mg, 0.96 mmol) and KBr (1.14 g, 9.6 mmol). Dropping funnels containing saturated Na₂CO₃ solution (300 ml) and 13% NaOCl solution (120 ml) were fixed to the flask. The carbonate solution was started on a rapid drip and the NaOCl was added at a slow drip (ca. 1 drop/second). After 100 ml of carbonate had been added, the pH was checked and more was added as necessary to maintain ca. pH 10. The temperature was maintained at 10° C. to 15° C. throughout. After 3 hr. starting material remained so more NaOCl (10 ml) was added rapidly. After 0.5 hr. the reaction was quenched with methanol (10 ml). The mixture was poured into a 4000 ml Erlenmeyer flask, submerged in an ice bath and adjusted to pH 3 with 6N HCl. The mixture was diluted with ethyl acetate and washed with 1 N HCl and 2× with distilled water allowing the layers to separate on the final wash. The organic layer was concentrated in vacuo to give the product as a white foam (38 g, 74%).

Quantitative ¹HNMR (500 MHz, d4-MeOH) using 2,3,4,5-tetrachloronitrobenzene (TCNB) internal standard relative to anomeric CH gives 94.8% of expected weight. Purity by TLC >95% (20% MeOH/DCM/2 drops HOAc, stain using 20% H₂SO₄/EtOH+heat) ¹H NMR (500 MHz, d₄-MeOH): δ=4.30 (d, 1H, J=9.5), 3.85 (m, 1H), 3.77 (d, 1H, J=7.5), 3.48-3.56 (m, 2H), 3.37 (t, 1H, J=11.5), 3.21 (t, 1H, J=9.5), 2.20 (t, 2H, J=9.5), 1.52-1.66 (m, 4H), 1.44 (s, 9H), 1.34-1.42 (br, 2H), 1.28 (s, 25H).

### Example 2. Dual Agonist Peptides—In Vitro Assays

Cellular assays were carried out using standard cellular assays (DiscoveRx, LeadHunter assays) using readout of cAMP stimulation or arrestin activation. Compounds were weighed precisely in an amount of approximately 1 mg and shipped to DiscoverX (Fremont, Calif.) for dilution and assay. The assay used were for the glucagon (human, cloned into CHO cells) and CLP-1 (human, cloned into CHO cells) receptors in cellular assays. Assays were carried out in the presence of 0.1% ovalbumin. Historically such assays have been carried out in the presence of 0.1% BSA, but for these compounds which bind very tightly to serum albumin (>99%) it can distort the results and make the compounds seem much less potent. Use of 0.1% ovalbumin can avoid this problem. The improvement seen upon use of ovalbumin can be seen as an indicator of relative tightness of serum albumin binding for the peptide.

TABLE 5

| | EC₅₀ cAMP | EC₅₀ cAMP | EC₅₀ cAMP | EC₅₀ cAMP |
|---|---|---|---|---|
| | GLP-1 R (pM) | glucagon R (pM) | GLP-1 R (pM) | glucagon R (pM) |
| Compound | 0.1% Ovalb | 0.1% Ovalb | 0.1% BSA | 0.1% BSA |
| EU-A1588 | 124 | 250 | 23 | 43 |
| EU-A1871 | 39 | 66 | 162 | 461 |
| EU-A1872 | 43 | 86 | 1266 | 2624 |
| EU-A1873 | 39 | 44 | 1,116 | 1,680 |
| semaglutide | 14.9 | >0.01 | 181 | N/A |

EU-A1588=SEQ ID NO: 2; EU-A1871=SEQ ID NO: 3; EU-A1872=SEQ ID NO: 4; EU-A1873=SEQ ID NO: 1; semaglutide=SEQ ID NO: 11.

Assays were carried out in the presence of 0.1% ovalbumin. Historically such assays have been carried out in the presence of 0.1% BSA, but for these compounds which bind very tightly to serum albumin (>99%) it can distort the results and make the compounds seem much less potent. Use of 0.1% ovalbumin can avoid this problem. The improvement seen upon use of ovalbumin can be seen as an indicator of relative tightness of serum albumin binding for the peptide, see table below.

TABLE 6

| | | EC50 cAMP | EC50 cAMP | EC50 cAMP | EC50 cAMP | Ovalbumin vs. BSA - | |
|---|---|---|---|---|---|---|---|
| | | GLP-1 R (pM) | glucagon R (pM) | GLP-1 R (pM) | glucagon R (pM) | fold improv/worsen | |
| Compound | Side chain | 0.1% Ovalb | 0.1% Ovalb | 0.1% BSA | 0.1% BSA | GLP-1 R | GCG R |
| EU-A1588 | melibiouronyl C16 | 124 | 250 | 23 | 43 | 5 | 6 |
| EU-A1871 | glucuronyl 15CO2H | 38.8 | 65.5 | 162 | 461 | 4 | 7 |
| EU-A1872 | glucuronyl 17CO2H | 42.5 | 86.4 | 1266 | 2624 | 30 | 30 |
| EU-A1873 | glucuronyl 17CO2H | 38.7 | 43.5 | 1,116 | 1,680 | 29 | 39 |
| semaglutide | (PEG)2-17CO2H | 14.9 | >0.01 | 181 | N/A | 12 | |

Effect of Replacement of BSA with Ovalbumin in Cellular Assay for Tight BSA Binders

Here one can see that the very tight serum albumin binders (with CO2H containing substituents, mimicking a fatty acid head group) show a substantial fold improvement upon replacement of BSA by ovalbumin, which does not bind fatty acid mimics appreciably. The degree of fold improvement can give a reading on tightness of binding to the fatty acid binding sites on BSA. Thus, semaglutide improves 12-fold (tight binding) while EU-A1873 improves from 30 to 40×, implying substantially increased serum albumin binding. This degree of serum albumin binding can be expected to result in a suppressed Cmax and prolonged duration of action, as is seen in the bioassays for SEQ ID NO: 1.

The data presented in Tables 5 and 6 above demonstrate that the tested compounds are agonists of both GLP-1R and GCGR (“dual agonists”), unlike semaglutide which shows high affinity biased towards GLP-1. This data also shows that SEQ ID NO: 1 is a dual agonist peptide with about equal affinity for GLP-1R and GCGR.

### Example 3. In Vivo Effects on Glucose, Body Weight, and Fat Loss

A. In vivo assays using db/db mice. About seventy five (75) BKS.Cg-m+/+Leprdb/J (Jackson Labs stock number 000642) male (“db/db”) mice at the age of 7-9 weeks of age were used in these studies, and maintained using standard animal care procedures. Studies initiated after one-week acclimation to facility conditions. On the morning of study day 0, mice were weighed and fasted for 4 hrs. Blood glucose was measured by glucometer using standard procedures. At least fifty-four (54) mice were selected based on body weights and those with blood glucose levels ≥300 mg/dL (i.e., diabetic) were randomly assigned into 6 groups (n=9). Groups were as follows: group 1, vehicle; group 2, semaglutide 3 nmol/kg; group 3, semaglutide 10 nmol/kg; group 4 SEQ ID NO: 1, one (1) nmol/kg; group 5, SEQ ID NO: 1, three (3) nmol/kg; group 6, SEQ ID NO: 1, 10 nmol/kg. Clinical observations were conducted at receipt, prior to randomization, and daily from Days 1 to 5. Body weights were measured and recorded at receipt, prior to randomization, and daily from Days 1 to 5. Food consumption was measured and recorded daily from Days 1 to 5. Blood samples for glucose analysis were collected pretest (Day −3) and at 0, 1, 4, 8, 24, 48, 72, 96 and 120 hours following the single dose of the indicated compound (e.g., SEQ ID NO: 1) on Day 1.

B. In vivo assays using “DIO JAX” mice. Eighty-one (81) 18 week-old male C57BL/6J mice, fed a high fat diet (Research Diets D12492) from the age of 6 weeks, were transferred to Jackson in vivo research laboratory (Sacramento, Calif.). The mice were ear-notched for identification and housed in individually and positively ventilated polycarbonate cages with HEPA filtered air at a density of up to 3 mice per cage. Cages were changed every two weeks. The animal room was lighted entirely with artificial fluorescent lighting, with a controlled 12 h light/dark cycle (6 am to 6 pm light). The normal temperature and relative humidity ranges in the animal rooms were 22±4° C. and 50±15%, respectively. The animal rooms were set to have 15 air exchanges per hour. Before study initiation, all mice continued on the high fat diet (60% kcal; D12492) and were acclimated for four weeks. On the morning of Study Day −1, baseline body composition was determined for each mouse via NMR analysis. Sixty-three (63) mice were grouped into seven groups (n=9). Remaining ungrouped mice were euthanized. Subcutaneous dosing of compounds was done on alternate days. On the morning of Study Day 0, pre-dose blood glucose measurements were taken via glucometer and the mice were dosed according to Table 7 below, with dose time recorded. Blood glucose measurements were taken at 1, 2, 4, 8, 10, and 24 hours post-dose. After study day 1, pre-dose blood glucose was measured on days 4, 7, 9, 11, 13, 17, 21 and 25. Body weights and clinical observations were recorded every 2 days. Food intake in all groups was measured daily, following dosing. First food intake measurement was on Study Day −1. Group 4 was pair-fed to Group 3 and Group 7 was pair-fed to Group 6. The amount of food for Groups 4 and 7 was dictated by the average amount of food consumed in the previous 24-hour window by Groups 3 and 6, respectively. Food intake for Groups 1, 2, 3, 5 and 6 were provided ad lib and measured daily. On Study Day 27, the mice were fasted for 5 hours and a glucose tolerance test (GTT) was performed. All mice were IP dosed with a bolus of glucose (2 g/kg) and blood glucose was assessed pre-dose and 15, 30, 60, 90, and 120 minutes post-dose. All blood glucose values were entered in the GTT Blood Glucose Log.

TABLE 7

| | | | Dosing | |
|---|---|---|---|---|
| Group | Treatment | N | Route | Dosing Frequency |
| 1 | Vehicle | 9 | SC | Every 2 days; |
| | | | | 0, 2, 4, 6, 8, 10, 12, 14, |
| | | | | 16, 18, 20, 22, 24 and 26 |
| 2 | Semaglutide | 9 | SC | Every 2 days; |
| | (6 nmole/kg) | | | 0, 2, 4, 6, 8, 10, 12, 14, |
| | | | | 16, 18, 20, 22, 24 and 26 |
| 3 | Semaglutide | 9 | SC | Every 2 days; |
| | (12 nmole/kg) | | | 0, 2, 4, 6, 8, 10, 12, 14, |
| | | | | 16, 18, 20, 22, 24 and 26 |
| 4 | No dosing, | 9 | N/A | N/A |
| | pair-fed to Group 3 | | | |
| 5 | MD-1373 | 9 | SC | Every 2 days; |
| | (6* nmole/kg) | | | 0, 2, 4, 6, 8, 10, 12, 14, |
| | | | | 16, 18, 20, 22, 24 and 26 |
| 6 | MD-1373 | 9 | SC | Every 2 days; |
| | (12* nmole/kg) | | | 0, 2, 4, 6, 8, 10, 12, 14, |
| | | | | 16, 18, 20, 22, 24 and 26 |
| 7 | No dosing, | 9 | N/A | N/A |
| | pair-fed to Group 6 | | | |

Note 1*
Mice in groups 5 and 6 were dosed at 3 nmole/kg and 6 nmole/kg on days 0, 2 and 4. Starting from day 8, mice in these groups were dosed at 6 nmole/kg and 12 nmole/kg respectively as indicated in the Table 1.
Note 2:
SEQ ID NO: 1 is referred to as MD-1373 in Table 7.

C. Glucose Control and Tolerance

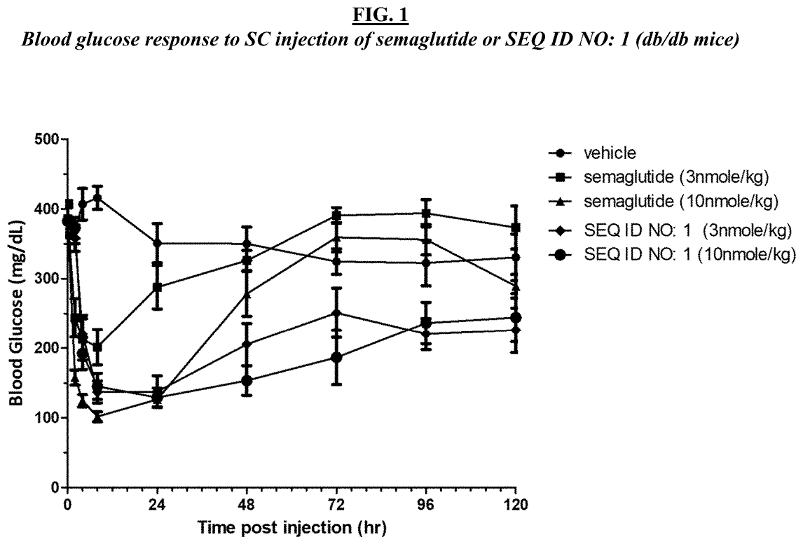

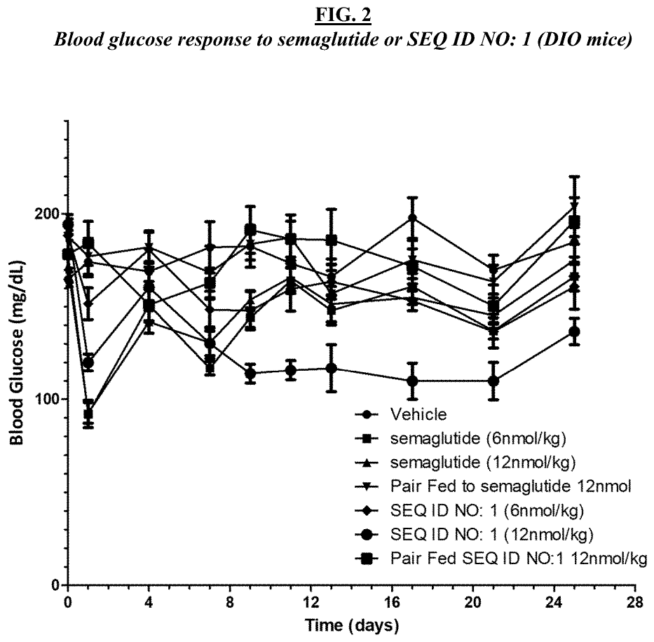

Using the db/db mouse model, glucose levels for semaglutide high dose were suppressed for 24 hrs, returning to pre-treatment levels by 48 hrs, while SEQ ID NO: 1 suppressing blood glucose beginning at 4 hours and extending to at least 96 hrs, and even up to 120 hours (FIG. 1). Thus, SEQ ID NO: 1 was found to exhibit an increased blood glucose response and a prolonged duration of action as compared to equimolar amounts of semaglutide in db/db mice. The onset-of-action of SEQ ID NO: 1 would be understood by those of ordinary skill in the art to be indicative of a likely reduction in acute gastrointestinal (GI) side effects observed as compared to using semaglutide. The onset-of-action of SEQ ID NO: 1 would also be understood by those of ordinary skill in the art to be indicative of a likely reduction in acute gastrointestinal (GI) side effects observed at a lower dose as compared to using semaglutide.

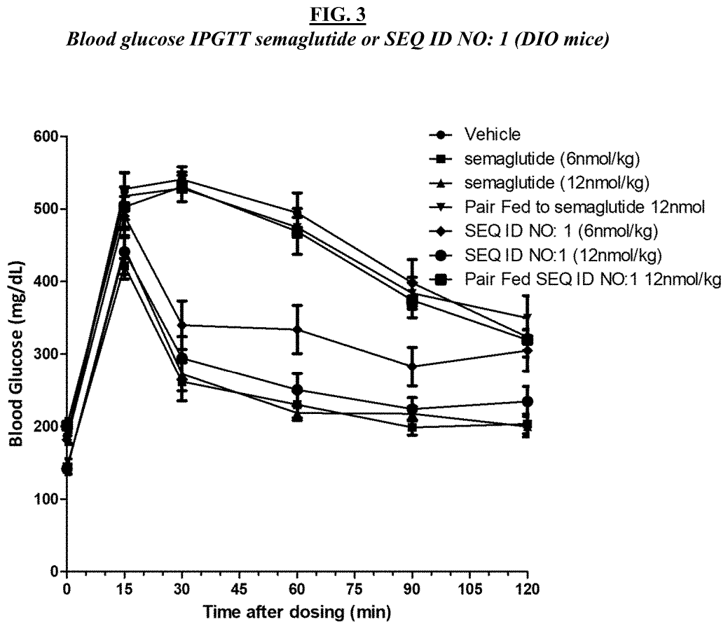

DIO JAX mouse studies also showed that blood glucose levels for the low (6 nmol/kg) and high (12 nmol/kg) doses of semaglutide were reduced to the normoglycemic range two hours post-dose, and remained suppressed in the normoglycemic range through day one (1) post-dose, but returned to hyperglycemic levels by day two (2) post-dose. Low- and high-dose (6 nmol/kg and 12 nmol/kg, respectively) SEQ ID NO:1 (“MD-1373”) suppressed blood glucose levels to the normoglycemic range by four (4) hours post-dose, the low dose remained suppressed through day 2 post-dose, and only returned to hyperglycemic range by day four (4) post-dose. Blood glucose levels in animals administered high-dose (12 nmol kg) SEQ ID NO: 1 were suppressed to the normoglycemic range from day seven (7) through the last measurement at day 26 (FIG. 2). For the other groups, there was a slight decrease, but blood glucose levels remained in the high hyperglycemic range throughout the remainder of the assay. This data indicates a lower dose of SEQ ID NO: 1 (As compared to an agonist with unbalanced affinity for GLP1R/GCGR) to achieve desired biological effects with a reduction in adverse events following administration to a mammal.

In addition, DIO JAX mice showed a large glucose excursion in response to a two (2) g/kg IP glucose challenge (intraperitoneal glucose tolerance test (IPGTT)). Both low- and high-dose SEQ ID NO: 1 groups exhibited a blunted glucose excursion, indicating good glucoregulatory effect. For instance, as shown as shown in FIG. 3, glucose tolerance was found to be similar between SEQ ID NO: 1 and semaglutide using the IPGTT in the DIO JAX mouse model. As shown therein, the IPGTT assay at day 27 showed similar results for high dose SEQ ID NO: 1 and semaglutide.

D. Body Weight and Fat Loss

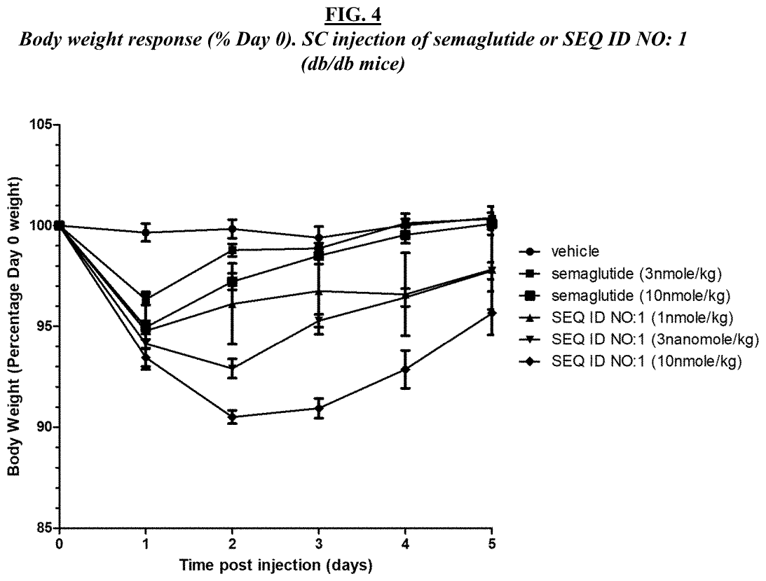

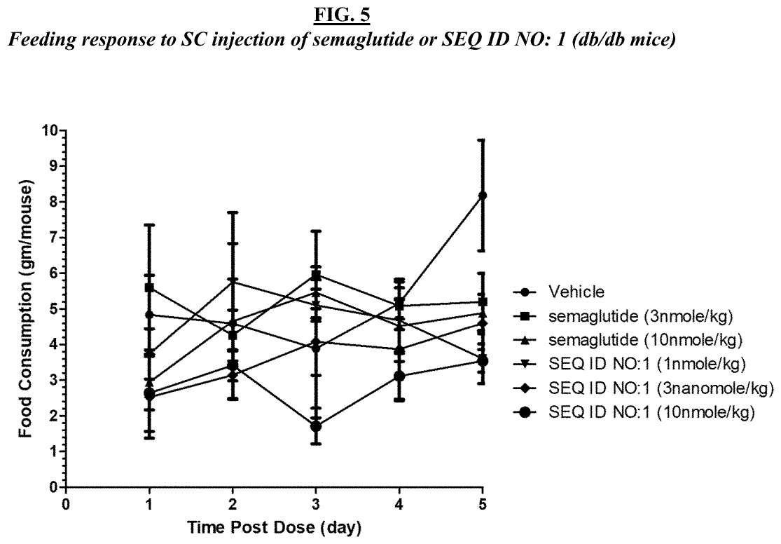

SEQ ID NO: 1 was found to result in greater weight loss as compared to semaglutide in BKS.Cg-m+/+Leprdb/J (Jackson Labs stock number 000642) (db/db) mice. Significant body weight changes were noted against vehicle for semaglutide and SEQ ID NO: 1 on day 1 post dosing and for mid and high dose of SEQ ID NO: 1 on Days 2 through 4 (FIG. 4). In the food consumption analysis, semaglutide high dose significantly suppressed feeding on day 1 post dose only, while SEQ ID NO: 1 was found to suppress feeding between days 1 through 4 (FIG. 5).

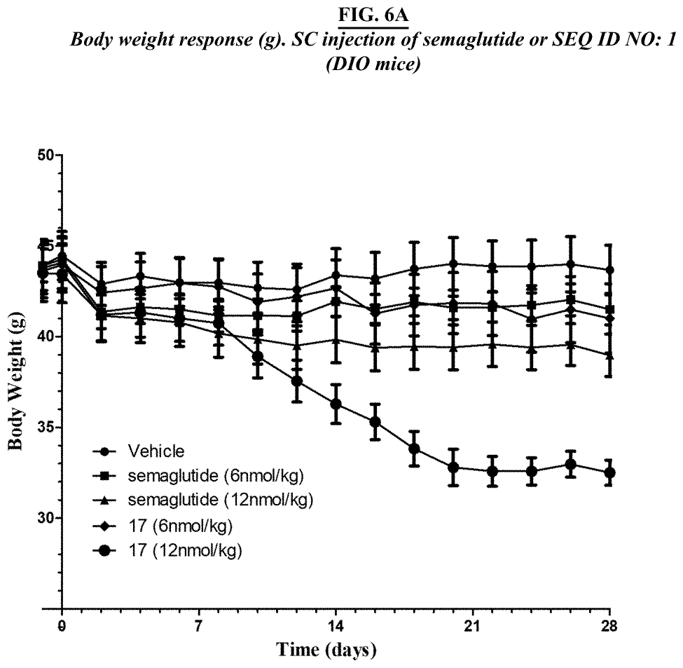

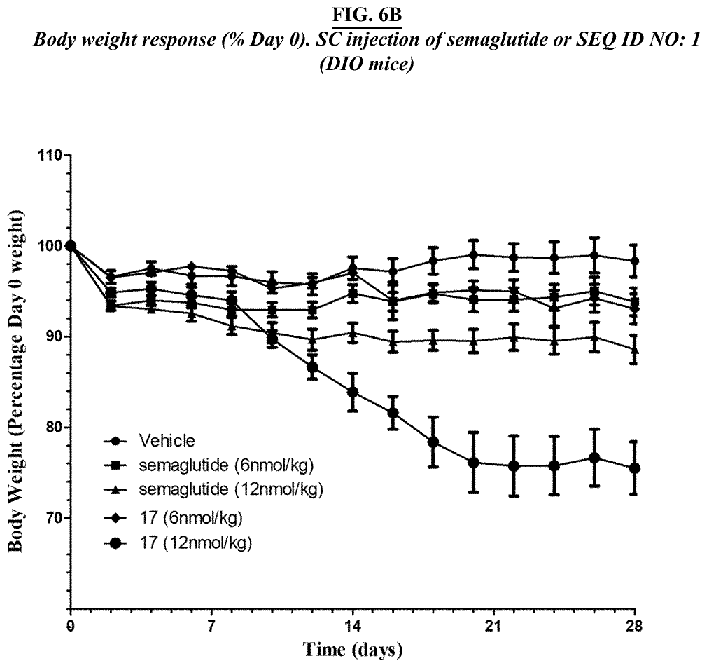

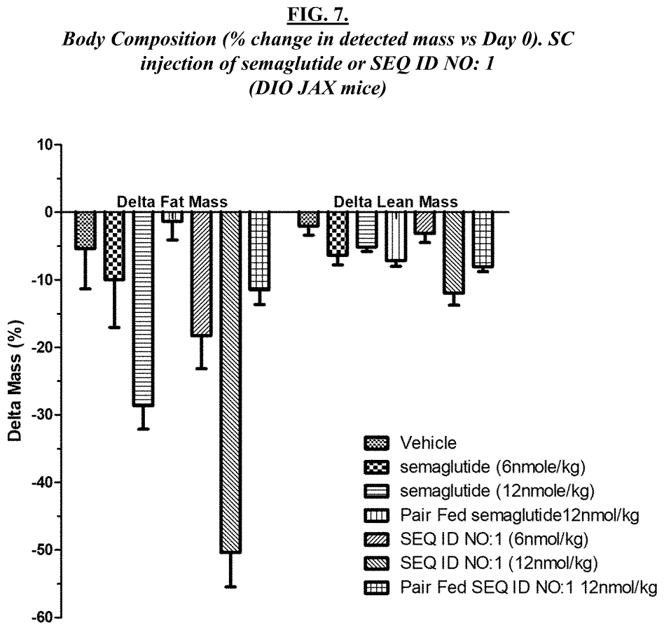

Glucagon co-agonism of SEQ ID NO: 1 was found to induce a very strong, stable weight loss of more than 25% (12 nmol/kg dose) in DIO JAX mice, more than twice that observed following semaglutide administration (e.g., 8-10%), despite the similarity in food intake between the groups (FIG. 6). Surprisingly, this data suggests SEQ ID NO: 1 operates by a second mechanism of action (e.g., acts on both sides of the “energy equation”, inducing both reduced food intake and increasing energy output). It is noted that on Day 8, SEQ ID NO: 1 groups of DIO JAX mice were switched from to a 6 to a 12 nmol/kg regimen to correct for pharmacodynamic (PD) differences between this DIO JAX mice population and db/db mice in which the earlier dose finding had been determined.

In addition, as shown in FIG. 7, SEQ ID NO: 1 nearly doubled the fat loss observed following semaglutide administration (51% vs. 28%, respectively (−6% for the vehicle control group)). Observed lean loss was about 12% for SEQ ID NO: 1 vs. 6% for semaglutide (−3% for the vehicle control group).

### Example 4. Pharmacokinetics

A. Mouse Studies

The in-life phase of the study was conducted at the Jackson Laboratory (Sacramento, Calif.) in sixty-seven C5713L6/J male mice (7-9 weeks of age) (diet-induced obese (DIO) JAX mice). The mice were ear notched for identification and housed in individually and positively ventilated polycarbonate cages with HEPA filtered air at a density of up to 4 mice per cage. The animal room was lighted entirely with artificial fluorescent lighting, with a controlled 12 h light/dark cycle (6 am to 6 pm light), The normal temperature and relative humidity ranges in the animal rooms were 22±4° C. and 50±15%, respectively. The animal rooms were set to have a minimum of 15 air exchanges per hour. Filtered tap water, acidified to a pH of 2.5 to 3.0, and standard rodent chow were provided ad libitum.

Both SEQ ID NO: 1 and semaglutide were formulated as 0.02 mg/mL in 50 mM phosphate buffer containing 0.05% tween 80 at pH ˜8. The dosing volume was 1.9365 and 5.8095 mL/kg for SEQ ID NO: 1 at 10 and 30 nmol/kg, respectively, and at 2.057 mL/kg for semaglutide at 10 nmol/kg. Three mice in unclosed Group 1 were bled at time zero only. In Group 2 (semaglutide; 10 nmol/kg SC), Group 3 (SEQ ID NO: 1; 10 nmol/kg SC), Group 4 (SEQ ID NO: 1; 10 nmol/leg IV), and Group 5 (SEQ H) NO: 1; 30 nmol/leg SC), blood samples were collected up to 120 hours post dose administration (n=4 per time point). Plasma concentrations of SEQ ID NO: 1 and semaglutide were determined using LC-MS/MS and the pharmacokinetic parameters were determined by non-compartmental analysis using WinNonlin.

Blood samples were collected at 1, 4, 8, 24, 48, 72, 96, and 120 hours post-dose administration. For Groups 2 to 5, four mice were bled at 2 time points with the second time point being terminal. At each time point, a minimum of −200 whole blood was collected via retro-orbital bleed or cardiocentesis. The blood samples were collected in K₂EDTA anticoagulant and centrifuged. The plasma (a minimum of 100 μL) was transferred to a tube and stored frozen until shipment to the bioanalytical lab for analysis by LC-MS/MS.

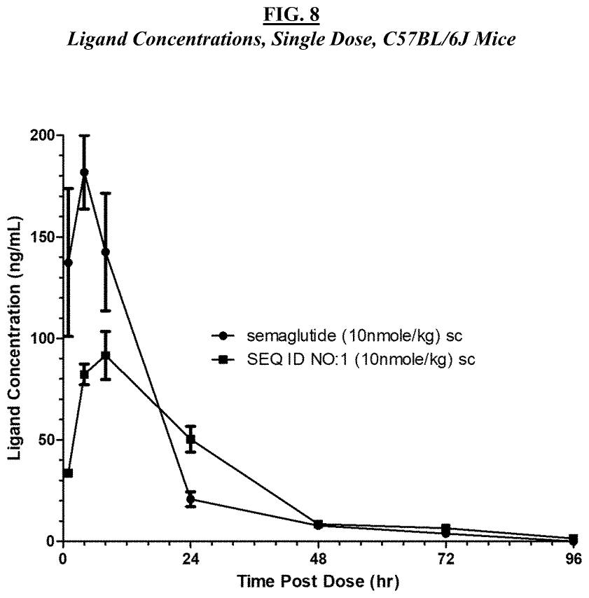

The determination of concentrations of SEQ ID NO: 1 and semaglutide in the plasma was conducted at the Climax Laboratories (San Jose, Calif.). A 100 μL aliquot of the plasma was mixed with 10 μL of internal standard (20 μg/mL standard in phosphate buffered saline) and then 300 μL of acetonitrile. The samples were vortexed and centrifuged. The supernatant was transferred to a clean 96-well plate for LC-MS/MS analysis. The data are presented in visual form in FIG. 8 with a tabular representation in Table 8.

TABLE 8
Non-Compartmental Pharmacokinetic Parameters of SEQ ID
NO: 1 and Semaglutide Following Subcutaneous or Intravenous
Administration to Male Mice (n = 4 per time point)

Compound

| | SEQ ID NO: 1 | | | Semaglutide |
|---|---|---|---|---|
| Dose (nmol/kg) | 10 | 10 | 30 | 10 |
| route | IV | SC | SC | SC |
| Cₘₐₓ | 79.5 | 23.7 | 76.9 | 44.2 |
| (nM) | | | | |
| Tₘₐₓ | 8 | 8 | 8 | 4 |
| (hr) | | | | |
| AUC₍₀₋ₜ₎ | 1500 | 687 | 1930 | 727 |
| (nM · hr) | | | | |
| AUC_{(0-∞)} | 1530 | 695 | 1950 | 755 |
| (nM · hr) | | | | |
| T_{1/2} | 14.7 | 15.4 | 10.0 | 20.0 |
| (hr) | | | | |
| MRT | 14.8 | 22.2 | 18.3 | 15.5 |
| (hr) | | | | |

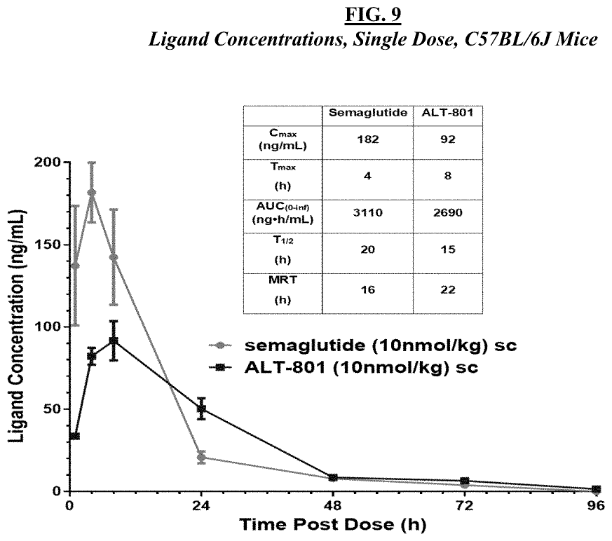

Following SC administration, as shown in FIG. 9, plasma levels of SEQ ID NO: 1 peaked later than semaglutide with a Tₘₐₓ of 8 and 4 hours, respectively. At 10 nmol/kg, the AUC of SEQ ID NO: 1 was comparable to that of semaglutide while the Cₘₐₓ of SEQ ID NO: 1 was 54% of that of semaglutide. The lowered Cmax with a similar AIX exhibited by SEQ H) NO: 1 considered a more favorable profile since it suggests a potential for lowered side effects since higher than therapeutic blood levels and peak to trough concentration ratios are minimized.

Overall, SEQ ID NO: 1 had a slightly longer MRT than semaglutide, 18.3 to 22.2 hours and 15.5 hours, respectively. Following SC dosing, the plasma concentrations of SEQ ID NO: 1 increased approximately dose-proportionally with a 3-fold increase in dose resulting in a 3.2- and 2.8-fold increase in Cₘₐₓ and AUC, respectively. Following IV administration, the plasma concentrations of SEQ ID NO: 1 increased with time with a Tₘₐₓ of 8 hours post dose. Since the plasma concentration-time profile suggested the IV dose may have been delivered perivascularly, instead of the intended intravascular injection, the bioavailability of SEQ ID NO: 1 following SC injection was not calculated.

A similar test was carried out using male C57BL6/J mice at The Jackson Laboratory-JAX West (Sacramento, Calif.). The pharmacokinetic (PK) parameters following a single subcutaneous (s.c). administration of ALT-801 (comprising SEQ ID NO: 1) or semaglutide (both 10 nmol/kg) were evaluated. Both compounds were formulated at 0.02 mg/mL in 50 mM phosphate buffer, 0.05% Tween 80 at pH ˜8. The dosing volume was approximately 2 mL/kg. Blood samples (˜200 μL) were collected at 1, 4, 8, 24, 48, 72, 96, and 120 hours post-dosing (n=4 per time point). Each mouse was bled at two time points and the second time point was a terminal bleed. Plasma concentrations of ALT-801 and semaglutide were determined using liquid chromatography coupled with tandem mass spectrometry (LC-MS/MS) with a limit of quantitation of 1.00 and 2.00 ng/mL for semaglutide and ALT-801, respectively. Non-compartmental PK analysis using WinNonlin was performed by using the mean concentrations at each sampling time point to report the maximum concentration (Cₘₐₓ), the time Cₘₐₓ was observed (Tₘₐₓ), the area under the plasma concentration curve from time zero to the last time point with measurable concentration (AUC₀₋ₜ), the plasma concentration-time curve from time zero to infinity (AUC₀₋₍ₓ₎), the terminal elimination half-life (T_{1/2}), and the mean residence time (MRT). The observed PK parameters for ALT-801 and semaglutide administered via the s.c. route at a dose of 10 nmol/kg are indicated in FIG. 9 (Tₘₐₓ=8 and 4 h, Cₘₐₓ=92 and 182 ng/mL, MRT=22 and 16 hr; respectively) and suggest a more measured and delayed approach to Cₘₐₓ in mice treated with ALT-801 relative to semaglutide. ALT-801 had a Cₘₐₓ 50% of, but AUC >86% of, the literature standard semaglutide's values. Elafibranor PK parameters were not assessed as it required the oral route of administration and was therefore not comparable to ALT-801 or semaglutide given by the s.c. route.

B. Miniature Swine Studies

The test animals were a total of four non-naïve male Yucatan miniature swine (Sus scrofa), housed singly. Body weights were from 73 to 75 kg. The housing room(s) were set to maintain a room temperature of 16 to 27° C. (61 to 81″F). Relative humidity was recorded. A 12-hr light/12-hr dark photoperiod was maintained. Room lights may have been turned on during the dark cycle to facilitate sample collection and/or other in-life activities. Animals were fed a maintenance amount of Purina S-9 swine diet. Clean, fresh water from an on-site deep water well was available ad libitum. General, in-cage observations were made at least twice daily (morning and evening) during the study period to assess general health, moribundity or mortality.

Following an acclimation period of twenty-two days each minipig was treated subcutaneously (behind cheek jowl) with SEQ ID NO: 1 at 20 nmol/kg, and pharmacokinetic blood samples were collected at −0.25, 2, 4, 6, 8, 12, 24, 48, 72, 96, 120, 168, 192, 216, 264, 312 and 360 hours post-dose. Following a two-week washout period, the same animals were administered SEQ ID NO: 1 intravenously and pharmacokinetic blood samples were collected at −0.25, 0.25, 0.5, 1, 2, 4, 8, 12, 24, 48, 72, 96, 120, 168, 192, 216, 264, 312 and 360 hour post-dose. Dose concentration was 5.5 mg/mL (dose volume 0.015 mL/kg) for both treatments.

Whole blood samples for pharmacokinetic analysis ˜3 ml/time point) in tubes containing K₂EDTA were collected via vascular access ports (VAP). Samples were maintained on wet ice until processing, ˜30 minutes or less post-collection. All samples were centrifuged for ˜15 minutes at ˜3000 rpm and ˜4° C. Resulting plasma was transferred evenly into two cryovials (primary and backup) and placed on dry ice. Plasma samples were stored frozen at ˜70° C., until primary samples were shipped for analysis.

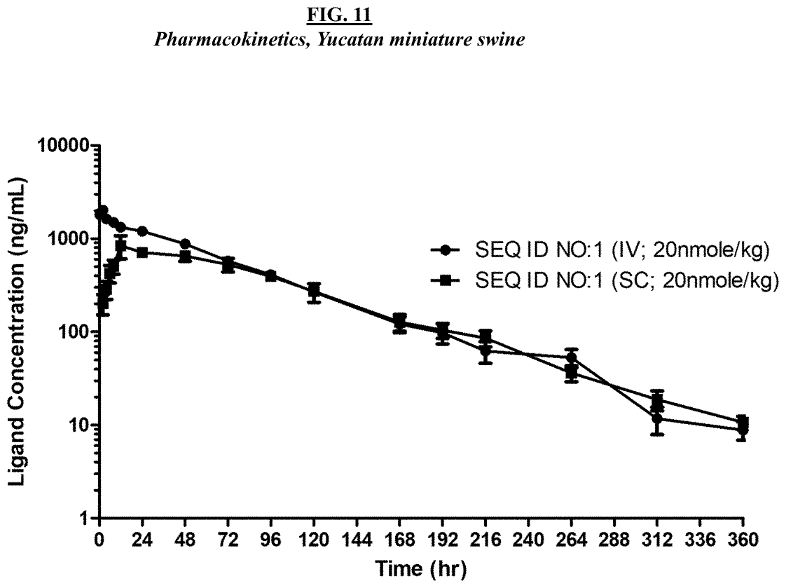

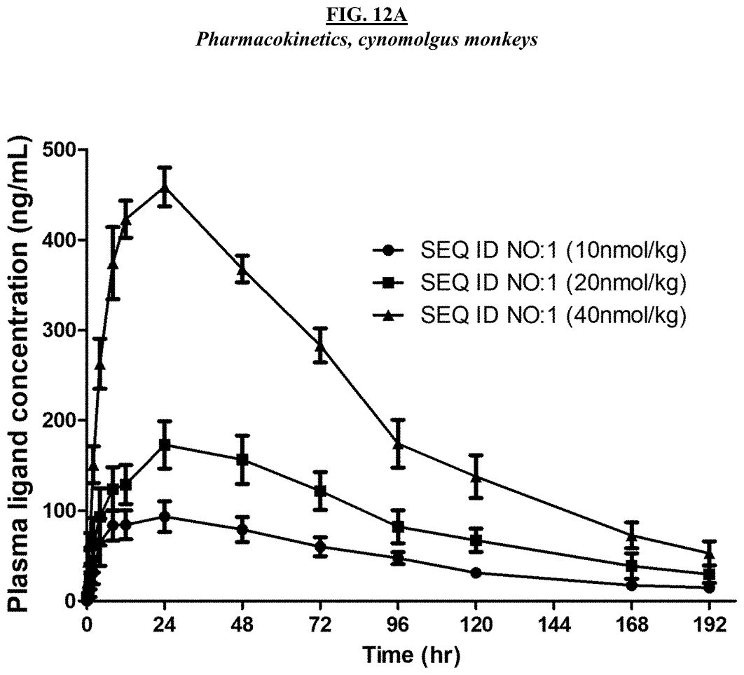

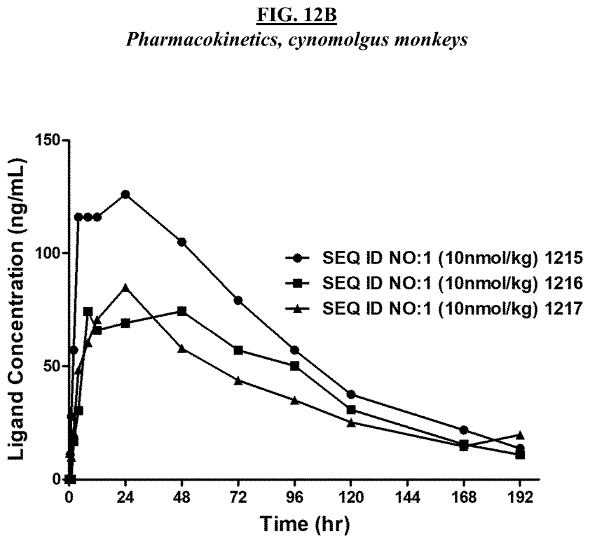

No abnormal clinical observations were observed during study conduct. The concentrations of the test articles are shown in FIG. 11.

It was also observed that, following SC administration of SEQ ID NO: 1, plasma levels of SEQ ID NO: 1 rose to a Cmax of 887 ng/mL at Tmax=52 hr., with a MRT of 86 hr. By contrast, semaglutide has a reported MRT of 64 hr. (Lau, J., et al. (2015) J Med Chem 58: 7370-80) in minipigs. This low Cmax and extended MRT again illustrates a prolonged duration of action relative to semaglutide, indicating a longer PD profile for SEQ ID NO: 1.

C. Rat Studies

1. Single Dose Protocol

Sixteen (+2 spares) male CRL:CD(SD) rats, approximately 250-300 g upon study-initiation, were received from the standing colony maintained at Charles River Labs. Animals were maintained on standard diet (Lab Diet C504). Food consumption was monitored on Study Days 1 through 7 by weighing the food and hopper together. Food and drinking water were provided ad libitum throughout the study with the exception of the overnight fasting periods occurring prior to dosing on Study Day −1. All animals were assigned into groups upon receipt.

On Study Day 1, all animals were administered a bolus dose of group dependent test article (TA) via subcutaneous inter(mid)-scapular injection. Individual animal body weights were recorded beginning on Day −1. Throughout dosing and at all sample collection time points, the animals were observed for any clinically relevant abnormalities. This study activity is described in more detail in Table 9.

TABLE 9
Study Activity

| Study Activity | | Timeline |
|---|---|---|
| Body Weights | | Pre Dose & Days 2, 3, 4, 5, 6, 7 |
| Food Intake | | Daily (Days, −1, 1, 2, 3, 4, 5, 6, 7) |
| Blood Collection | | Day 1 (2, 4, 8 hrs) |
| (Whole blood processed to plasma) | | Day 2 (24 hrs) |
| Anti-coagulant | K2EDTA | Day 3 (48 hrs) |
| Volume/Time | 300 ul | Day 4 (72 hrs) |
| point | | Day 6 (120 hrs) |
| | | Day 7 (144 hrs) →Max |
| | | Obtainable |
| | | *hrs = post dose |

Following administration of TA on Study Day 1, a 300 uL sample of whole blood was collected into K₂EDTA tubes via the indwelling jugular vein catheter (JVC) at the timepoints listed A maximum obtainable volume of blood was collected via cardiac puncture for the final timepoint (144-hrs post dose) following CO₂ euthanasia. Whole blood samples were stored on wet ice for no longer than 30 minutes prior to centrifugation at 2200×g for 10 minutes at 5° C.±3° C. The resulting plasma was then pipetted into polypropylene tubes and stored nominally in a freezer set to maintain a temperature of −80° C. until transfer to Climax Laboratories (San Jose, Calif.) for pharmacokinetic analysis. SEQ ID NO: 1 was administered in formulation buffer (0.050% (w/w) polysorbate 20, 0.300% (w/w) methylparaben, 0.348% (w/w) Arginine, 4.260% (w/w) Mannitol in DI water) at target dose levels of 0.03 mg/kg, 0.1 mg/kg, or 0.2 mg/kg.

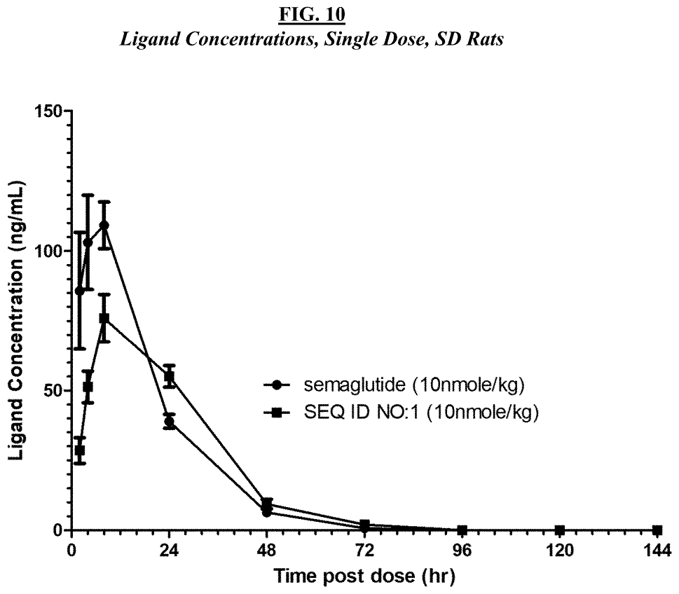

Following SC administration, as shown in FIG. 10, plasma levels of SEQ ID NO: 1 and semaglutide rose rapidly in the rats. Semaglutide peaked with a Tₘₐₓ near 8 hrs. while the concentration of SEQ ID NO: 1 was still rising at 8 hrs., suggesting the true Tmax would be at a later time point. By the next timepoint, 24 hr, it had peaked and is declining somewhat but is still higher than semaglutide. At 10 nmol/kg, the AUC of SEQ ID NO: 1 (2350 ng·hr/mL) was comparable (93%) to that of semaglutide (2530 ng·hr/mL) while the Cₘₐₓ of SEQ ID NO: 1 was 54% of that of semaglutide. The lowered Cmax with a similar AUC exhibited by SEQ ID NO: 1 is considered a very favorable profile since it suggests a potential for lowered side effects, since higher than therapeutic blood levels and peak to trough concentration ratios are minimized. Overall, SEQ ID NO: 1 had a longer MRT than semaglutide, 20.6 hours vs. 15.4 hours for semaglutide, respectively.

2. Repeat Dose Protocol in Rats

The purpose of this study was to evaluate the toxicity and toxicokinetics of the test article, ALT-801, when administered daily via subcutaneous injection to rats for at least 6 weeks and to assess the reversibility, persistence, or delayed occurrence of any effects after a 4-week recovery phase. Animal receiving 0.03 mg/kg/day ALT-801 were treated for the entire study duration without any issues. In contrast, animals treated at doses ≥0.09 mg/kg/dose were placed on significant dosing holidays during the first 3 weeks of the study because of significant ALT-801 dose-related food consumption and associated body weight suppression during that time period. Dose formulation analyses revealed that significant out of specification results for all ALT-801 dose formulations were plausibly the root cause for the exaggerated effects observed during the first 3 weeks of the study in Group 3 and 4. Dose formulation analysis issues were resolved by end of Week 3, and treatment was resumed starting on Day 22 for animals in Groups 3 and 4, and the study duration subsequently extended for an additional 2 weeks of treatment (terminal necropsy on Day 57). Group 3 animals were treated with 0.03 mg/kg/day ALT-801 on Days 22 and 23 and then received their targeted dose of 0.09 mg/kg/dose, once every other day (Q2D) for the remainder of the study. Group 4 animals were treated with 0.09 mg/kg/day on Days 22 and 23 and then received their targeted dose of 0.15 mg/kg/dose as 3 days on/4 days off for the remainder of the study. Accordingly, ALT-801 was overall administered at 0.03 mg/kg/day daily for 8 consecutive weeks (Group 2), at 0.09 mg/kg/dose once every other day (Q2D) for 5 consecutive weeks (Group 3), or at 0.15 mg/kg/dose administered as 3 days on/4 days off for 5 consecutive weeks.

TABLE 10

| | No. of TK Animals | | Dose Level |
|---|---|---|---|
| Group | Male | Female | (mg/kg/dose) |
| 1 (Control)^{a} | 3 | 3 | 0 |
| 2 (Low) | 6 | 6 | 0.03 |
| 3 (Mid) | 6 | 6 | 0.03^{c}/0.09^{c, e} |
| 4 (High) | 6 | 6 | 0.09^{b}/0.15^{b, d} |

^{a}Group 1 was administered vehicle control article only.
^{b}Group 4 animals were administered 0.15 mg/kg/dose. Starting on Day 14, Group 4 animals were administered 0.09 mg/kg/dose. Starting on Day 16, Group 4 animals were dose-escalated to 0.15 mg/kg/dose. Starting on Day 22 of the dosing phase, Group 4 animals were administered 0.09 mg/kg/dose. Starting on Day 24 of the dosing phase, Group 4 animals were dose-escalated to 0.15 mg/kg/dose until the end of the dosing phase.
^{c}Group 3 animals were administered 0.09 mg/kg/dose. Starting on Day 22 of the dosing phase, Group 3 animals were administered 0.03 mg/kg/dose. Starting on Day 24 of the dosing phase, Group 3 animals were dose-escalated to 0.09 mg/kg/dose until Day 35 of the dosing phase. Group 3 animals were not dosed on Day 36 of the dosing phase.
^{d}Starting on Day 32 of the dosing phase, Group 4 animals were dosed for three days (doses on Days 32-34), and then placed on dosing holiday for four days. This dosing regimen continued through the remainder of the dosing phase (doses on Days 39-41, 46-48, 53-55).
^{e}Starting on Day 37 of the dosing phase, Group 3 animals were administered 0.09 mg/kg/dose once every other day (Days 37, 39, 41, 43, 45, 47, 49, 51, 53, and 55) throughout the dosing phase.

Blood samples were collected from three toxicokinetic animals/sex/group/time point in Groups 2 through 4 on Day 1, Groups 3 and 4 on Day 55, and Group 2 on Day 56 predose and at approximately 1.5, 3, 6, 12, 24, 48 (Days 55 and 56 only), and 72 (Days 55 and 56 only) hours postdose. Blood samples were also collected from three toxicokinetic animals/sex/group/time point in the vehicle control group on Days 1 and 56 predose and at approximately 3, 12, 24 (Day 1 only), and 48 (Day 56 only) hours postdose. Blood samples were processed to plasma and were analyzed for ALT-801 at Covance-Madison and the results were used for the generation of this toxicokinetic report.

TABLE 11
Summary of the ALT-801 Toxicokinetic Parameters in Rat Plasma

| | Dose | Dose Level^{a} | | Cₘₐₓ | Tₘₐₓ | AUC₀₋₂₄ | AUC₀₋₇₂ | AUC₀₋₁₆₈ | t_{1/2} |
|---|---|---|---|---|---|---|---|---|---|
| Day | Group | (mg/kg/dose) | Sex | (ng/mL) | (h) | (h*ng/mL) | (h*ng/mL) | (h*ng/mL) | (h) |
| 1 | 2 | 0.03 | M | 47.2 | 24.0 | 734 | NR^{b} | NR^{c} | NR |
| | | | F | 50.1 | 24.0 | 828 | NR^{b} | NR^{c} | NR |
| | | | MF | 48.7 | 24.0 | 781 | NR^{b} | NR^{c} | NR |
| | 3 | 0.09 | M | 134 | 12.0 | 2430 | NR^{b} | NR^{c} | NR |
| | | | F | 79.5 | 12.0 | 1430 | NR^{b} | NR^{c} | NR |
| | | | MF | 107 | 12.0 | 1930 | NR^{b} | NR^{c} | NR |
| | 4 | 0.15 | M | 250 | 12.0 | 4400 | NR^{b} | NR^{c} | NR |
| | | | F | 275 | 12.0 | 4920 | NR^{b} | NR^{c} | NR |
| | | | MF | 263 | 12.0 | 4660 | NR^{b} | NR^{c} | NR |
| 55 | 3 | 0.09 | M | 182 | 24.0 | 3100 | 6820 | NR^{c} | NR |
| | | | F | 115 | 24.0 | 2130 | 4400 | NR^{c} | NR |
| | | | MF | 148 | 24.0 | 2620 | 5610 | NR^{c} | NR |
| | 4 | 0.15 | M | 496 | 24.0 | 10400 | 21500 | NR^{c} | NR |
| | | | F | 490 | 24.0 | 10500 | 22200 | NR^{c} | NR |
| | | | MF | 493 | 24.0 | 10400 | 21800 | NR^{c} | NR |
| 56 | 2 | 0.03 | M | 86.7 | 12.0 | 1770 | 3180 | 3330 | 15.1 |
| | | | F | 108 | 12.0 | 2090 | 3540 | 3600 | 11.2 |
| | | | MF | 97.1 | 12.0 | 1930 | 3360 | 3460 | 13.1 |

NR Not reported due to an inability to characterize the elimination phase.
NR^{b} Not reported due to the lack of a measurable concentration at 72 hours postdose.
NR^{C} Not reported due to the lack of a measurable concentration at 168 hours postdose.
Notes:
AUC₀₋₁₆₈ was calculated using extrapolation and should be interpreted with caution. Combined male and female (MF) parameters were calculated by combining concentration data for all animals (male and female) at each dose level on each interval and using these data as a separate composite profile for TK analysis. These parameters are not an average of the values calculated for males and females separately.
^{a}Animals were dosed once daily for at least 8 weeks (dosing phase). Group 3 animals were not dosed on Day 36. Starting on Day 37, Group 3 animals were dosed every other day (doses on Days 37, 39, 41, 43, 45, 47, 49, 51, 53, and 55) throughout the dosing phase. Starting on Day 32 of the dosing phase, Group 4 animals were dosed for three days (doses on Days 32-34), and then placed on dosing holiday for four days. This dosing regimen continued through the remainder of the dosing phase (doses on Days 39-41, 46-48, 53-55).

Sex differences in ALT-801 Cₘₐₓ, AUC₀₋₂₄, AUC₀₋₇₂, or AUC₀₋₁₆₈ values were less than 2-fold. Exposure, as assessed by ALT-801 Cₘₐₓ and AUC₀₋₂₄ values, increased with the increase in dose level from 0.03 to 0.15 mg/kg/dose on Day 1. The increases in ALT-801 Cₘₐₓ and AUC₀₋₂₄ values were generally dose proportional on Day 1. Potential accumulation of ALT-801 was observed after multiple doses in rats.

D. Single Dose Cynomolgus Monkey Studies

The purpose of this study was to determine the pharmacokinetics of SEQ ID NO: 1 after a single subcutaneous dose to cynomolgus monkeys (three (3) monkeys per dose group). No serious adverse events were noted in the animals during the study duration.

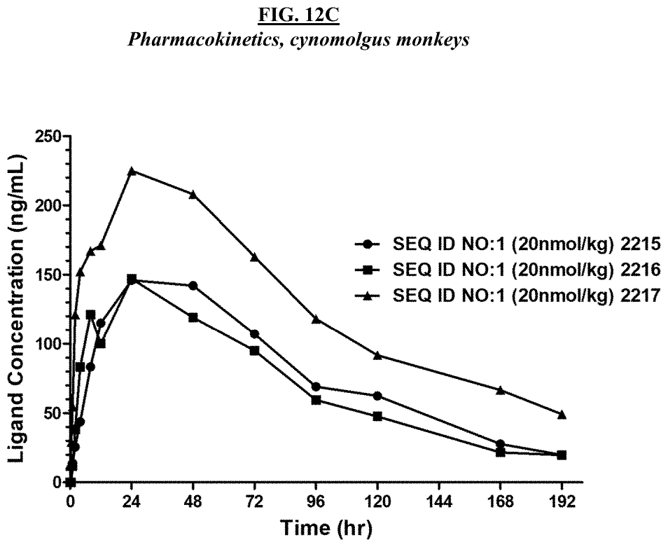

As shown in Table 13 below and FIG. 12, escalating doses of SEQ ID NO: 1 in formulation buffer (0.050% (w/w) polysorbate 20, 0.300% (w/w) methylparaben, 0.348% (w/w) Arginine, 4.260% (w/w) Mannitol in DI water) exhibit the pharmacokinetic parameters shown in Table 10 when tested in using Cynomolgus monkey model (SC administration) as measured over a time period of 192 hours post-dose.

TABLE 13

| | SEQ ID NO: 1 dose (mg/kg) | | |
|---|---|---|---|
| | 0.039 | 0.078 | 0.154 |
| Cₘₐₓ | 95.1 | 173 | 467 |
| (ng/mL) | | | |
| Tₘₐₓ | 32 | 24 | 20 |
| (hr) | | | |
| AUC₍₀₋₁₉₂₎ | 9340 | 17800 | 42200 |
| hr*ng/mL | | | |
| T_{1/2} (hr) | 59.1 | 55.6 | 52.3 |

FIG. 12B illustrates the plasma concentration of SEQ ID NO: 1 on day 9 following administration of ALT-801 in animals (labeled 1215, 1216 and 1217 in FIG. 12B) administered 10 nmol/kg SEQ ID NO: 1 (as ALT-801). Animal 1215 was found to have slightly unformed stool on day 9 after treatment (thus unlikely to be ALT-801 related), and to exhibit Cmax of 126 ng/mL (33 nM) as compared to the 80 ng/mL average of the other two animals (1216 and 1217) in this study. This data indicates that the biologically effective level of ALT-801 is probably <5 nM SEQ ID NO: 1. This low dose group (10 nmol/kg) shows no evidence for vomiting (0/3); unclear whether “unformed stool, scant” is compound related. The Cmax for the animal with the unformed stool is 158% of the average for the other two animals. All animals show blood levels >5 nM throughout 120 hr.

FIG. 12C illustrates concentration of SEQ ID NO: 1 on day 9 following administration of ALT-801 in animals (labeled 2215, 2216 and 2217 in FIG. 12C) administered 20 nmol/kg SEQ ID NO: 1 (as ALT-801). Animal 2217 exhibited some vomiting on day 2 following administration, and to exhibit Cmax of 225 ng/mL (58 nM) as compared to the 147 ng/mL average of the other two animals in this study. This data also indicates that the biologically effective level of ALT-801 is probably <5 nM SEQ ID NO: 1. This mid dose group (20 nmol/kg) shows slight evidence (⅓) for vomiting. The Cmax for the animal vomiting is 153% of the average for the other two animals. All animals show blood levels >5 nM throughout 192 hr.

FIG. 12D illustrates concentration of SEQ ID NO: 1 on day 9 following administration of ALT-801 in animals (labeled 3215, 3216 and 3217 in FIG. 12D) administered 40 nmol/kg SEQ ID NO: 1 (as ALT-801). All three animals exhibited some vomiting that may be ALT-801 and Cmax-related. The average Cmax for this group was of 467 ng/mL (121 nM). This data also indicates that the biologically effective level of ALT-801 is probably <5 nM SEQ ID NO: 1. This high dose group (40 nmol/kg) shows strong evidence (3/3) for vomiting. The Cmax for this relatively homogeneous group is 467 ng/mL (121 nM). All animals show blood levels >10 nM throughout the assay (192 hr).

The evidence for GI side effects supports our suggestion that it is Cₘₐₓ related, at least in NHPs (non-human primates). If the biologically effective blood level is <5 nM, 10 nmol/kg may be a higher dose than needed. Dose accumulation is anticipated for treatment with ALT-801. In embodiments, provided is a pharmaceutical formulation comprising ALT-801 as the API configured for subcutaneous administration providing a Cmax of 150-200 ng/ml wherein adverse GI side effects are reduced or eliminated but ALT-801 is effective at reducing blood glucose levels and/or for treating obesity.

E. Multiple Dose Cynomolgus Monkey Studies

1. 6-Week Repeat Dosing Studies in Cynomolgous Monkeys

Study Objectives

The objective of this study was to evaluate the toxicity and toxicokinetics of ALT-801 (comprising SEQ ID NO: 1) when administered once weekly for at least 6 weeks (total of six doses) via subcutaneous injection to cynomolgus monkeys and to assess the reversibility, persistence, or delayed occurrence of any effects after a 4-week recovery phase. The study was conducted by Covance.

Animals

Male and female cynomolgus monkeys (28 animals/sex; Macaca fascicularis) of Asian origin were received from Envigo Global Services Inc. (previously Covance Research Products) in Alice, Tex. Animals were acclimated to the test facility for at least 30 days prior to initiation.

At initiation of dosing, animals were 31 to 54 months old. On the day prior to initiation of dosing, body weights ranged from 2.2 to 4.2 kg for males and from 2.2 to 3.2 kg for females.

Study Design

Male and female cynomolgus monkeys were assigned to five groups, and doses were administered as indicated in the following table. Animals were dosed via subcutaneous injection into the dorsal region on Days 1, 8, 15, 22, 29, and 36 of the dosing phase at a volume of 2.0 mL/kg. The vehicle control article was F58 Formulation Buffer, which consisted of 0.050% (w/w) polysorbate 20, 0.348% (w/w) arginine, 4.260% (w/w) mannitol in deionized water (pH 7.7±0.1).

| | No. of | | Dose | Pre-dosing | Dosing phase | | | | | | | Recovery |
|---|---|---|---|---|---|---|---|---|---|---|---|---|
| | Animals (b) | | Level | ≥30 days | Day | Day | Day | Day | Day | Day | Day | phase (b) |
| Group | Male | Female | (mg/kg) | acclimation | 1 | 8 | 15 | 22 | 29 | 36 | 48 | 28 days |
| 1 (Control) (a) | 5 | 5 | 0 | E | D | D | D | D | D | D | E | E |
| 2 (ALT-801 | 5 | 5 | 0.03 | E | D | D | D | D | D | D | E | E |
| 0.03 mg/kg) | | | | | | | | | | | | |
| 3 (ALT-801 | 5 | 5 | 0.06 | E | D | D | D | D | D | D | E | E |
| 0.06 mg/kg) | | | | | | | | | | | | |
| 4 (ALT-801 | 5 | 5 | 0.18 | E | D | D | D | D | D | D | E | E |
| 0.18 mg/kg) | | | | | | | | | | | | |
| 5 (ALT-801 | 5 | 5 | 0.25 | E | D | D | D | D | D | D | E | E |
| 0.25 mg/kg) | | | | | | | | | | | | |

(a) Control = vehicle control article only.
(b) Two animals designated for recovery evaluation underwent 4 weeks of recovery following the completion of the dosing phase.
D = Dosing;
E = Evaluation

Assessment of toxicity was based on mortality, clinical observations, body weights, qualitative food consumption, ophthalmic observations, electrocardiographic (ECG) measurements, neurological examinations, qualitative respiration rates, and clinical and anatomic pathology. Blood samples were collected for toxicokinetic evaluations.

Description of the Test Article

| Test Article | Storage | Lot | Retest Date^{b} | Purity^{a} |
|---|---|---|---|---|
| ALT-801 | Frozen (−10 to −30° C.) protected from | S548 | 19 Nov. 2020 | 95.22% |
| | light with dessicant | | | |

^{a}Purity was determined by high-performance liquid chromatography on an anhydrous basis. A correction factor of 1.192 was assigned.
^{b}Assigned per Covance SOP as 365 days from receipt

Description of the Vehicle Control Article

The vehicle control article was F58 Formulation Buffer, which was comprised of 0.050% (w/w) polysorbate 20, 0.348% (w/w) arginine, 4.260% (w/w) mannitol in deionized water (pH 7.7±0.1).

Formulation of the Test Article

Test article formulations were prepared in vehicle control article at least once weekly according to the mixing procedure and were apportioned for use. Dose concentrations were corrected for lot-specific purity using a correction factor of 1.192. The pH of each test article formulation was adjusted, as necessary, to pH 7.7±0.1 using dilute hydrochloric acid or sodium hydroxide. The prepared test article formulations were sterile filtered using 0.2 μm polyvinylidene difluoride filters (PVDFs); post filtration handling was performed using aseptic technique.

Formulation of the Vehicle Control Article

Vehicle control article formulations were prepared at least once weekly by Covance according to the mixing procedure and were apportioned for use. The prepared vehicle control article formulations were sterile filtered using a 0.2-μm PVDF; post filtration handling was performed using aseptic technique, and the filtered solution was dispensed into dosing aliquots for Group 1. All concentration values of ALT-801 in the vehicle control group were below the lower limit of quantitation (<4.00 ng/mL).

Dosing

The dose sites were in the dorsal scapular region of each animal. Doses were rotated between the sites. The dose sites were as follows: Dose Site A: Upper left scapular region, Dose Site B: Upper right scapular region, Dose Site C: Lower left scapular region, Dose Site D: Lower right scapular region The following animals were not dosed due to body weight loss, body condition score, and veterinary recommendation on the days listed in the following table.

| Animal | Group/Sex | Day(s) not Dosed |
|---|---|---|
| P0302 | 4/M | 8 |
| P0605 | 2/F | 22 |
| P0701 | 3/F | 8 |
| P0702 | 3/F | 8 and 22 |
| P0703 | 3/F | 8 |
| P0704 | 3/F | 8 |
| P0705 | 3/F | 8, 15, and 22 |
| P0801 | 4/F | 8 |
| P0802 | 4/F | 8 |
| P0803 | 4/F | 8 |
| P0804 | 4/F | 8 and 22 |
| P0805 | 4/F | 8 |
| P0901 | 5/F | 8 |
| P0902 | 5/F | 8 |
| P0903 | 5/F | 8 |
| P0904 | 5/F | 8 |
| P0905 | 5/F | 8 |

F = Female; M = Male.

Toxicokinetic Analysis

The toxicokinetic analysis included parameters listed in the following table.

| Parameter | Description |
|---|---|
| Cₘₐₓ | Maximum observed concentration |
| Tₘₐₓ | Time of maximum observed concentration |
| AUC₀₋ₜ | Area under the curve from time 0 to the time of the last measurable concentration, |
| | calculated using the linear trapezoidal rule |
| AUC₀₋₁₆₈ | Area under the curve from time 0 to 168 hours, calculated using the linear trapezoidal rule |
| AUC₀₋₃₁₂ | Area under the curve from time 0 to 312 hours, calculated using the linear trapezoidal rule |
| | (recovery animals only) |
| t_{1/2} | Elimination half-life, calculated as ln(2)/λ_{z} |

A summary of the Mean ALT-801 Toxicokinetic Parameters in Monkey Plasma are presented in the table below. All concentration values of ALT-801 in the vehicle control group were below the lower limit of quantitation (<4.00 ng/mL).

| | Dose | Dose Level | | Cₘₐₓ | Tₘₐₓ | AUC₀₋ₜ^{a} | AUC₀₋₁₆₈ | AUC₀₋₃₁₂ | t_{1/2} |
|---|---|---|---|---|---|---|---|---|---|
| Day | Group | (mg/kg) | Sex | (ng/mL) | (h) | (h*ng/mL) | (h*ng/mL) | (h*ng/mL) | (h) |
| 1 | 2 | 0.03 | M | 59.9 | 48.0 | 6070 | 6070 | NA | 56.9 |
| | | | F | 61.8 | 48.0 | 6140 | 6140 | NA | 56.1 |
| | | | MF | 60.9 | 48.0 | 6100 | 6100 | NA | 56.5 |
| | 3 | 0.06 | M | 91.9 | 48.0 | 8990 | 8990 | NA | 51.0 |
| | | | F | 107 | 48.0 | 11600 | 11600 | NA | 55.9 |
| | | | MF | 99.5 | 48.0 | 10300 | 10300 | NA | 53.7 |
| | 4 | 0.18 | M | 302 | 48.0 | 32300 | 32300 | NA | 60.4 |
| | | | F | 323 | 48.0 | 32300 | 32300 | NA | 62.3 |
| | | | MF | 312 | 48.0 | 32300 | 32300 | NA | 61.6 |
| | 5 | 0.25 | M | 290 | 24.0 | 32000 | 32000 | NA | 69.0 |
| | | | F | 342 | 48.0 | 32800 | 32800 | NA | 59.4 |
| | | | MF | 316 | 36.0 | 32400 | 32400 | NA | 63.0 |
| 36 | 2 | 0.03 | M | 64.1 | 48.0 | 7890 | 6520 | 8080 | 50.9 |
| | | | F | 65.9 | 30.0 | 7110 | 5970 | 7210 | 60.4 |
| | | | MF | 64.9 | 48.0 | 7500 | 6280 | 7640 | 54.5 |
| | 3 | 0.06 | M | 137 | 24.0 | 14500 | 12600 | 14700 | 58.4 |
| | | | F | 143 | 48.0 | 16100 | 14200 | 16100 | 62.2 |
| | | | MF | 140 | 36.0 | 15300 | 13400 | 15400 | 60.3 |
| | 4 | 0.18 | M | 401 | 24.0 | 39500 | 38900 | 39500 | 63.4 |
| | | | F | 383 | 48.0 | 34700 | 34900 | 34700 | 61.2 |
| | | | MF | 392 | 36.0 | 37100 | 36900 | 37100 | 62.3 |
| | 5 | 0.25 | M | 505 | 48.0 | 59300 | 47400 | 59300 | 60.7 |
| | | | F | 619 | 48.0 | 65400 | 61400 | 65400 | 63.2 |
| | | | MF | 562 | 48.0 | 62300 | 54400 | 62300 | 61.8 |

M = Males,
F = Females,
MF = Males and Females

Veterinary Treatments and Examinations

No ALT-801 related veterinary health issues were noted. No remarkable ophthalmic observations were noted during the dosing phase. Based on these results, no ophthalmic examinations were performed during the recovery phase. No remarkable neurological observations were noted during the dosing or recovery phase. Electrocardiographic examinations show no ALT-801-related changes in PR interval, QRS duration, QT interval, QTc interval, or heart rate were observed approximately 24 hours postdose on Day 1 or 36 of the dosing phase. No abnormal ECG waveforms or arrhythmias were observed during the qualitative assessment of ECGs.

Clinical Laboratory Evaluations

No ALT-801-related findings were observed in hematology, coagulation, clinical chemistry, or urinalysis test results. No ALT-801-related changes in organ weights were noted at the terminal or recovery necropsies. No ALT-801-related macroscopic findings were observed at the terminal or recovery sacrifices. No ALT-801-related microscopic findings were observed in animals at the terminal or recovery sacrifices.

Change in Body Weights

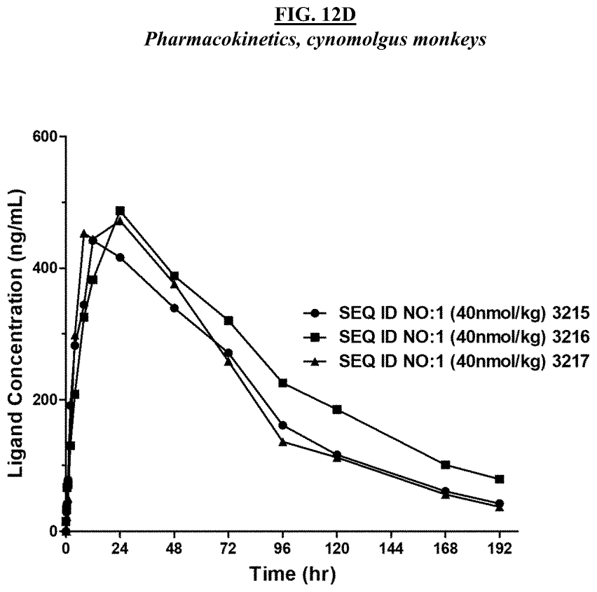

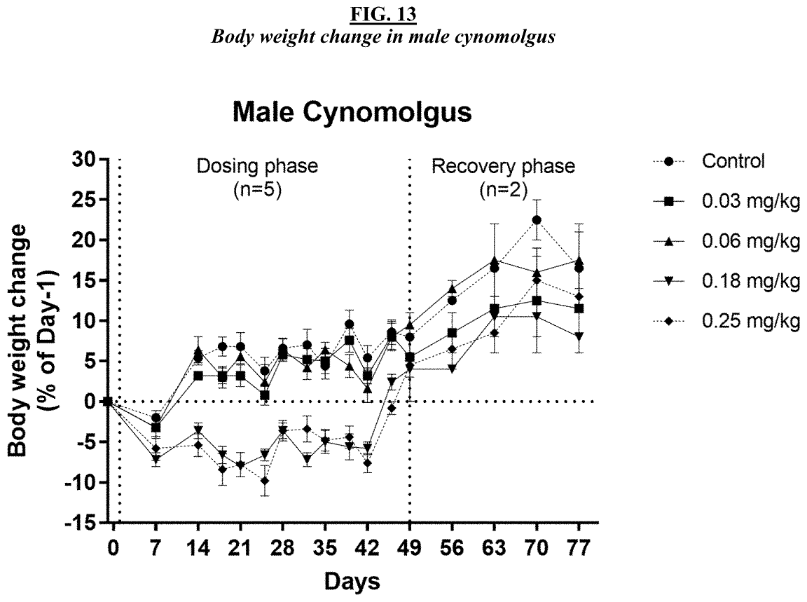

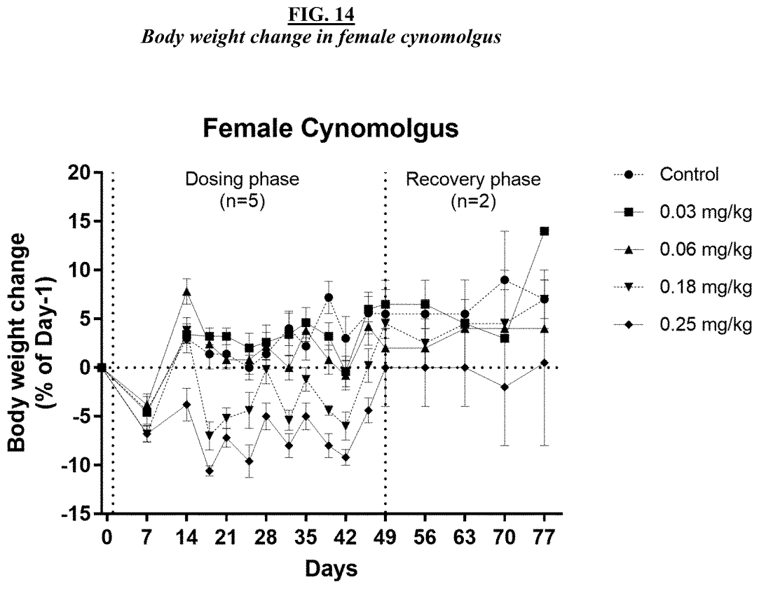

Body weights were recorded for animals four times during the predose phase, on Day −1 of the dosing phase (day prior to dose initiation), and weekly thereafter (based on Day −1) to Day 14 of the dosing phase. Starting on Day 14 of the dosing phase, body weights were collected twice weekly (based on Day 14) through to the end of the dosing phase. Body weights were collected on Days 1, 8, 15, 22, and 28 of the recovery phase. Data presented in FIG. 13 and FIG. 14 represent body weight change as a % of Day-1 in males and females respectively. At the two highest dose of ALT-801 (0.18 mg/kg and 0.25 mg/kg), significant weight loss up to 10% was observed during the dosing period in both males and/or females.

Clinical Observations

No ALT-801-related mortality or effects on neurological observations, ECGs, clinical pathology, organ weight, or macroscopic or microscopic examinations occurred during the dosing or recovery phase.

ALT-801-related clinical observations for females administered ≥0.03 mg/kg/dose included low food consumption. No ALT-801-related clinical observations were noted for males administered ≥0.03 mg/kg/dose. ALT-801-related, lower food consumption was observed for females administered ≥0.03 mg/kg/dose. No ALT-801-related changes in food consumption were observed for males administered ≥0.03 mg/kg/dose. Lower food consumption was observed on Days 19 and 36 of the dosing phase for females administered ≥0.03 mg/kg/dose ALT-801, with a dose-responsive increase in incidence.

Vomitus was observed once on Day 2 of the dosing phase in one female (Animals P0701) administered 0.18 mg/kg/dose and one female (Animal P0901) administered 0.25 mg/kg/dose. This observation did not persist and did not display a dose-responsive increase in incidence. As there was no dose responsive increase incidence for vomitus and these observations did not persist, therefore this was not considered an ALT-801-related clinical observation.

No ALT-801-related clinical observations were noted during the recovery phase.

One female (Animal P0604) administered 0.03 mg/kg was sacrificed at an unscheduled interval on Day 26 of the recovery phase. Clinical observations noted for this animal included hypoactive and hunched, with pale mucous membranes; rough haircoat; thin appearance; and dark dried feces on the tail, with liquid feces in the pan while not being commingled. This unscheduled sacrifice had no relationship to ALT-801, as Animal P0604 was in the recovery phase, and the clinical observations observed for this animal were not observed for other animals administered ALT-801.

Other clinical observations included swollen tail, scabs, abnormal skin color, liquid/non-formed feces, abnormal color pelage, thinning pelage, and red discharge from the vulva. These appeared rather infrequently, were transient, or were with comparable incidences as controls; therefore, they were not considered ALT-801 related.

### Conclusion

In conclusion, male and female monkeys were administered vehicle control article or 0.03, 0.06, 0.18, or 0.25 mg/kg/dose ALT-801 via subcutaneous injection once weekly.

As shown in FIGS. 13 and 14, the two highest dose of ALT-801 tested in the study (0.18 mg/kg and 0.25 mg/kg) lead to significant weight loss up to 10% during the dosing period in both males and/or females. This effect was not associated with any mortality or gastrointestinal events deemed to be related to the treatment at all doses tested.

No adverse, ALT-801 related findings occurred during the dosing or recovery period and the no observed adverse effect level (NOAEL) is 0.25 mg/kg/dose. This dose level corresponded to mean peak concentration (Cmax) and area under the concentration time curve (AUC) values of 562 ng/mL and 62300 h*ng/mL, respectively.

F. Summary of Example 4 rat and monkey data: These multidose studies showed no significant adverse events (AEs) in rats or cynomolgus monkeys. Reduced food consumption and weight loss, which were expected pharmacologic properties of ALT-801, were noted at the mid and high doses, but no ALT-801 related vomitus was observed. The high doses of 0.45 mg/kg/week and 0.25 mg/kg/week were established as the no adverse effect levels (NOAEL) in rats and monkeys, respectively. Safety pharmacology assessments, which were embedded in the general toxicology studies, were devoid of neurological, cardiac, or respiratory findings. As noted, reduced food consumption and weight loss observations were expected on-target effects of GLP-1 and glucagon agonism. These effects were more pronounced in rats compared to monkeys, possibly related to the more frequent dose cycle (QD initially) corresponding to the shorter t_{1/2} in rats. On an exposure basis, both Cₘₐₓ and area under the plasma concentration-time curve (AUC₀₋₁₆₈ₕ) (ie, over the dosing interval) were remarkably similar in rats dosed at 0.15 mg/kg for 3 days on and 4 days off each week (weekly dose of 0.45 mg/kg/week) and monkeys dosed at 0.25 mg/kg/week once weekly. In rats, Cₘₐₓ and AUC₀₋₁₆₈ achieved approximately 500 ng/mL and 42,600 ng*h/mL, respectively. Likewise, exposures in monkeys were 5560 ng/mL and 54,400 ng*h/mL, respectively.

### Example 5. Murine Non-Alcoholic Steatohepatitis (NASH)

In the DIO-NASH mouse study a total of 5 DIO-NASH groups (n=12) of male C57BL/6J mice were fed the Amylin High Fat Diet with 40% fat (including trans-fat), 18% fructose, 2% cholesterol diet for 29+ weeks. All mice entering the experiment have been pre-biopsied, stratified based on liver biopsy (only animals with fibrosis 1 or above and steatosis 2 or above are included) animals stratified into groups based on Col1a1 immunostaining. For a total of 12 weeks of QD dosing animal groups were: 1) Vehicle, 2) SEQ ID NO: 1, 5 nmol/kg (SC, QD), 3) SEQ ID NO: 1, 10 nmol/kg (SC, QD), 4) Elafibranor, 78 μmol/kg (PO, QD), 5) semaglutide, 10 nmol/kg (SC, QD). Body weight (BW) was measured daily for the entire study period, food intake daily for the first 14 days then weekly until study end. Terminal plasma was measured for ALT/AST/TG/TC levels. Terminal liver removal and sampling was carried out for pre to post NAFLD Activity Score (NAS; HE staining) including Fibrosis Stage (Picrosirius red, PSR). Terminal histology was carried out for steatosis, Col1a1 and galectin-3 quantitation. Terminal liver workup included TG+TC (extraction and measurement). Terminal liver biopsies were set up in: 1) 4% PFA for histology, 2) fresh frozen liver for biochemistry, 3) fresh frozen liver for RNA extraction and RNAseq.

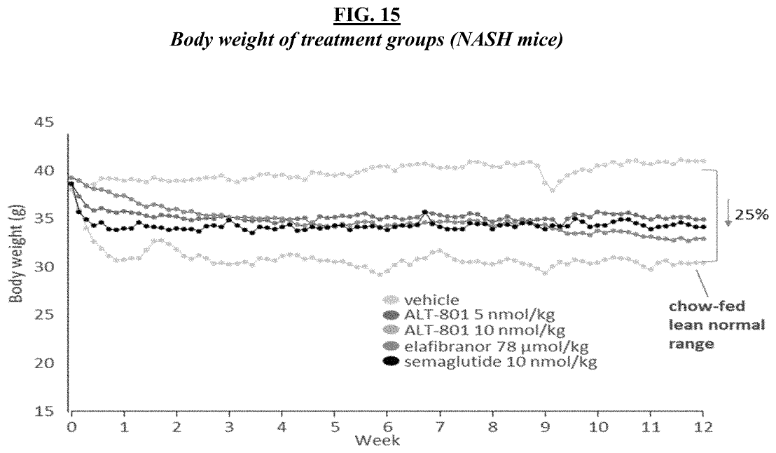

Treatment with ALT-801 (pharmaceutical formulation comprising SEQ ID NO: 1) was shown to decrease body weight in the NASH mouse model, treatment with ALT-801 and semaglutide caused body weights to rapidly and dose-responsively decrease, which stabilized for the remainder of the study (FIG. 15). Treatment with ALT-801 (5 nmol/kg and 10 nmol/kg), as well as elafibranor (78 μmol/kg) and semaglutide (10 nmol/kg), resulted in statistically significantly decreased body weight compared to NASH control (p≤0.001). The weight loss achieved in animals treated with ALT-801 was dose-dependent and reached −25% within 4 weeks of administration, approximately twice the weight loss induced by semaglutide, at an equimolar dose. Importantly, ALT-801 (10 nmol/kg) decreased the body weight for the group to the lean normal body weight range for this mouse strain (˜30 g), then maintained this range. On Day 63 (Week 9 of treatment), the vehicle group was inadvertently given a single dose of 10 nmol/kg ALT-801, resulting in a rapid decline in weight and subsequent recovery to vehicle trend line over a period of ˜10 days.

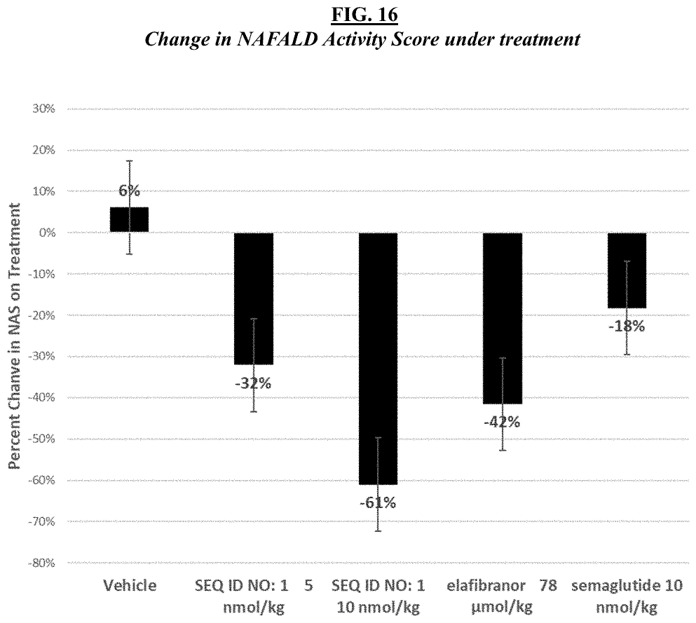

SEQ ID NO: 1 was also shown to exhibit a superior NAFLD activity score (NAS) reduction as compared to elafibranor and semaglutide. See FIG. 16. As shown therein, 5 nmol/kg SEQ ID NO: 1 exhibited a 32% reduction and 10 nmol/kg SEQ ID NO: 1 exhibited a 61% reduction, as compared to 42% for elafibranor and 18% for semaglutide, compared to the start of treatment (Day 0). The control group experienced a 6% increase. The NAS score improved in all treatment groups at the end of the treatment period (FIG. 15). The percent change in NAS score achieved by the elafibranor and semaglutide treatment groups were significantly less than the percent change achieved in the ALT-801 10 nmol/kg group (both p<0.0001). All animals in the ALT-801 10 nmol/kg group achieved NAS scores ≤3.

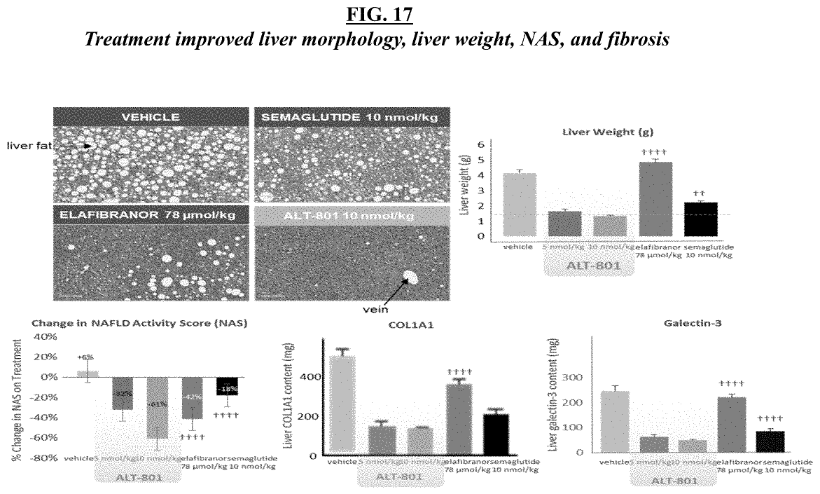

As shown herein, then, at the end of the treatment period, there was a reduction in the fat content of the livers of mice treated with low and high dose ALT-801 to that of the lean normal range (FIG. 17). Low and high dose treatment with ALT-801 resulted in significantly decreased liver weight as compared to NASH vehicle control, semaglutide, and elafibranor (p<0.01; FIG. 17). The mean liver weights of mice treated with elafibranor and semaglutide were statistically significantly higher than the liver weight in high dose (10 nmol/kg) ALT-801 treated mice (p<0.0001 and p<0.01, respectively). The liver weight of both groups treated with ALT-801 was similar to that of chow-fed lean normal mice.

Treatment with ALT-801 (pharmaceutical formulation comprising SEQ ID NO:1) was also found to lead to greater beneficial effects on fibrosis, as measured by liver Col1A1 and Galectin-3 content, compared to elafibranor, semaglutide, or NASH vehicle control. Low and high dose treatment with ALT-801 resulted in significantly lower terminal liver Col1A1 and Galectin-3 levels as compared to NASH vehicle control, elafibranor, and semaglutide (p<0.0001; FIG. 17). The mean liver Col1A1 level of mice treated with elafibranor was statistically significantly higher than the liver Col1A1 in high dose (10 nmol/kg) ALT-801 treated mice (p<0.0001). The mean liver Galectin-3 levels of mice treated with elafibranor and semaglutide were statistically significantly higher than the liver Galectin-3 in high dose (10 nmol/kg) ALT-801 treated mice (both p<0.0001).

Treatment with ALT-801 (pharmaceutical formulation comprising SEQ ID NO: 1) was also found to normalize liver triglycerides (TG), total cholesterol (TC), and plasma ALT. Low and high dose treatment with ALT-801 resulted in significantly lower liver TG (p<0.01) and TC (p<0.0001) levels as compared to NASH vehicle control, semaglutide, and elafibranor (FIG. 18). The mean liver TG levels of mice treated with elafibranor and semaglutide were statistically significantly higher than the liver TG in high dose (10 nmol/kg) ALT-801 treated mice (p≤0.01 and p≤0.0001 respectively; one-way ANOVA with Dunnett's adjustment for multiplicity). Similarly, the mean liver TC levels of mice treated with elafibranor and semaglutide were statistically significantly higher than the liver TC in high dose (10 nmol/kg) ALT-801 treated mice (both p<0.0001).

Low and high dose treatment with ALT-801 resulted in significantly lower terminal plasma AST levels compared to NASH vehicle control (p<0.001) as well as significantly lower terminal plasma ALT levels as compared to NASH vehicle control, elafibranor, and semaglutide (p<0.01; FIG. 18). The mean liver ALT level of mice treated with elafibranor and semaglutide was statistically significantly higher than the plasma ALT in high dose (10 nmol/kg) ALT-801 treated mice (p<0.0001 and p<0.01, respectively), which was within the normal range for this strain.

RNA sequencing showed that treatment with SEQ ID NO:1 was superior to treatment with elafibranor or semaglutide, resulting in the profound suppression of inflammatory and profibrotic gene expression, particularly in the stellate cells pathway responsible for fibrotic lesion development.

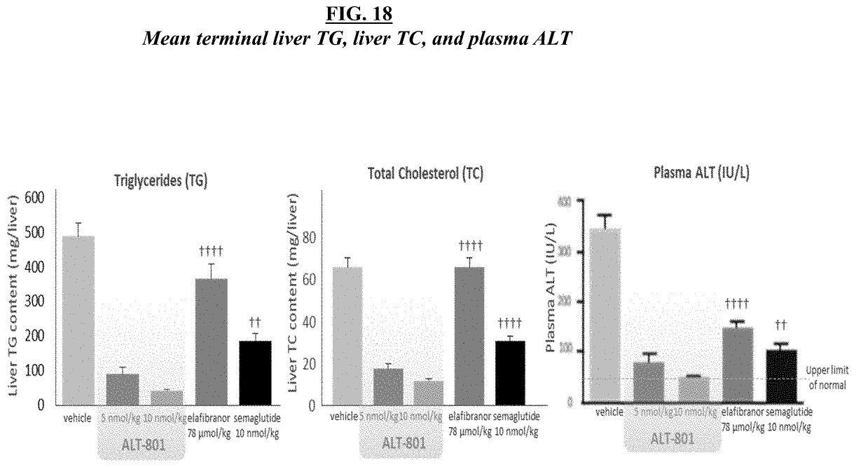

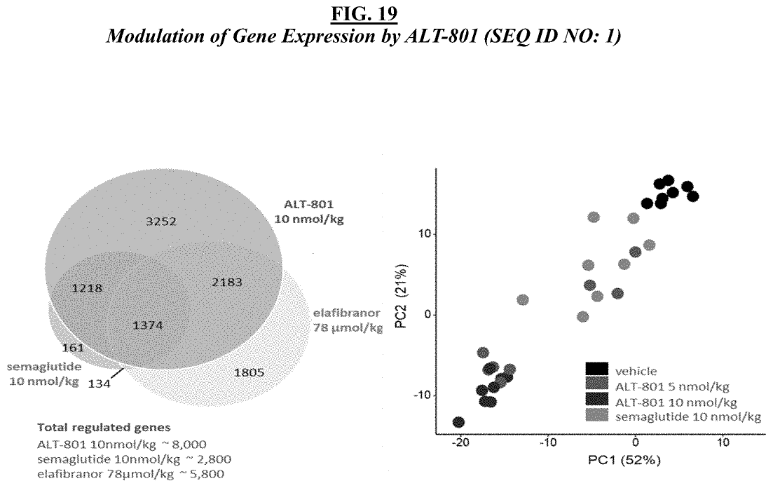

The high dose ALT-801 (pharmaceutical formulation comprising SEQ ID NO: 1) treatment group displayed the highest number of differentially expressed genes (˜8000) compared to either elafibranor (˜5800) or semaglutide (˜2800) (FIG. 19). Principal component analysis of the 500 most variable liver genes was performed which resulted in clear treatment-related clustering of the samples (FIG. 19). PC1 explained 52% of the variability and PC2 explained 21% of the variability.

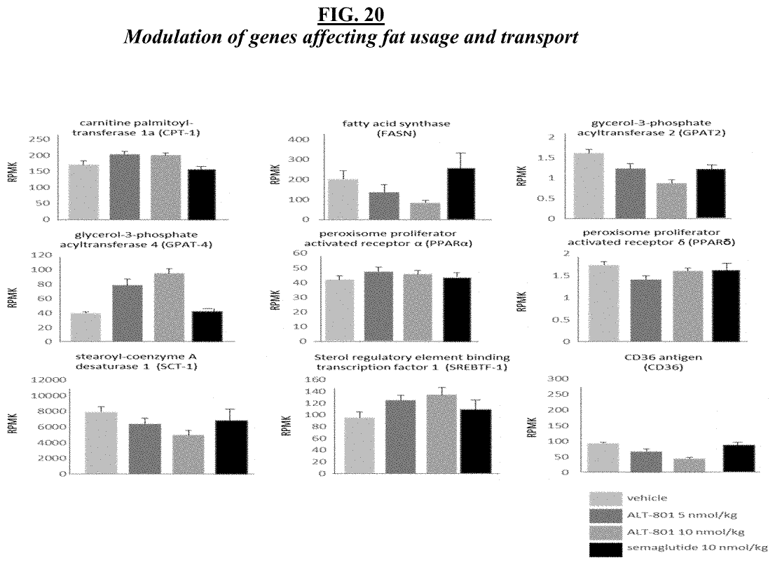

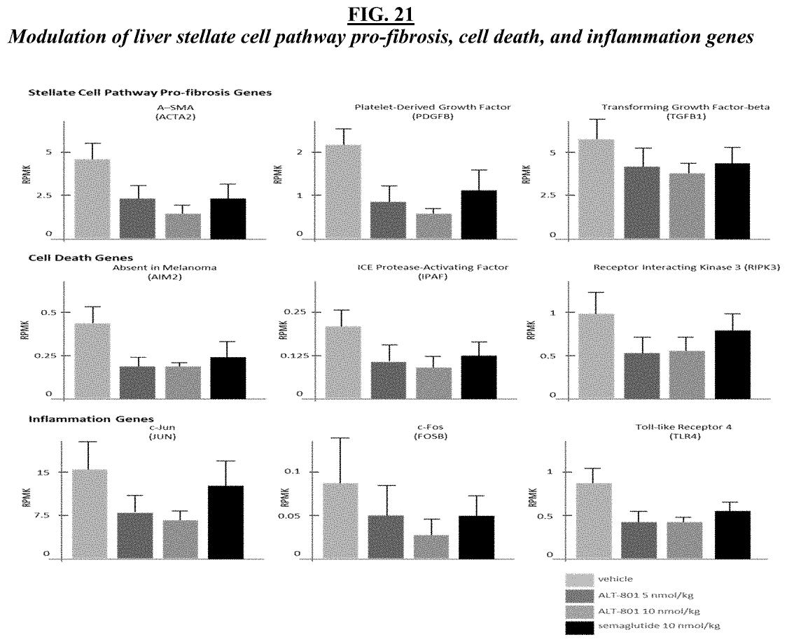

Treatment of NASH mice with 10 nmol/kg ALT-801 resulted in modulation of genes affecting fat usage and transport, including statistically significantly increased expression level of carnitine palmitoyl-transferase 1a (CPT-1) (p<0.05), glycerol-3-phosphate acyltransferase 4 (GPAT-4) (p<0.001), and sterol regulatory element binding transcription factor 1 (SREBTF-1) (p<0.05) compared to NASH vehicle control after correction for gene-wise multiple testing (FIG. 20). Treatment of NASH mice with the lower dose of ALT-801 (5 nmol/kg) also resulted in increased expression of CPT-1 (p<0.05) and GPAT-4 (p<0.001) (FIG. 18). Expression of fatty acid synthase (FASN) (p<0.05), glycerol-3-phosphate acyltransferase 2 (GPAT2) (p<0.001), stearoyl-coenzyme A desaturase 1 (SCT-1) (p<0.05), and CD36 antigen (CD36) (p<0.001) was decreased in mice treated with ALT-801 10 nmol/kg compared to NASH vehicle control after correction for gene-wise multiple testing (FIG. 20). CD36 expression was also significantly lower in mice treated with ALT-801 5 nmol/kg (p<0.05) (FIG. 20). The gene expression changes observed in mice following semaglutide treatment were not statistically significant; however, the elafibranor group had significantly lower GPAT2 (p<0.001) and GPAT4 (p<0.001) relative to NASH vehicle controls.

Treatment of NASH mice with ALT-801 resulted in suppression of stellate cell pathway pro-fibrosis genes. The myofibroblast proliferation and stellate cell markers A-SMA (ACTA2), platelet-derived growth factor (PDGFB), and transforming growth factor-beta (TGFB1) (FIG. 20) were statistically significantly decreased in the treatment groups given ALT-801 low or high dose compared to NASH vehicle control (all p<0.01, after correction for gene-wise multiple testing). Expression of A-SMA (p<0.001) and TGFB1 (p<0.05) also statistically significantly decreased in NASH mice treated with semaglutide, while expression of PDGF (p<0.01) statistically significantly decreased in NASH mice treated with elafibranor.

Treatment of NASH mice with ALT-801 resulted in suppression of cell death genes. The hepatocellular cell death and pyroptosis markers absent in melanoma (AIM2), ICE protease-activating factor (IPAF), and receptor interacting kinase 3 (RIPK3) (FIG. 20) were statistically significantly decreased in the treatment groups given ALT-801 low or high dose compared to NASH vehicle control (all p<0.01, after correction for gene-wise multiple testing). Expression of AIM2 (p<0.01) also statistically significantly decreased in NASH mice treated with semaglutide. No statistical differences in cell death genes were noted in treated with elafibranor.

Treatment of NASH mice with ALT-801 resulted in suppression of liver inflammation genes. The pro-inflammatory signaling markers c-Jun (JUN), c-FOS (FOSB), and toll-like receptor 4 (TLR4) (FIG. 20) were statistically significantly decreased in the treatment groups given ALT-801 low or high dose when compared to NASH vehicle control, with the exception of c-FOS in the ALT-801 low dose group (all p<0.01, after correction for gene-wise multiple testing). Expression of TLR4 (p<0.01) also statistically significantly decreased in NASH mice treated with semaglutide. No statistically significant changes in FOSB, JUN, or TLR4 genes were noted in NASH mice treated with elafibranor.

### Example 6. Pharmacodynamic (PD) and Pharmacokinetic (PK) Profiles and Weekly Dosing

This example relates to a series of peptide analogues with varying balance of receptor agonistic activity at the human GLP-1R and GCGR, and analogues having a duration of action suggesting suitability for once weekly (QW) administration in patients, including but not limited to SEQ ID NO. 1 as in ALT-801. A comparison of certain peptide analogues of this disclosure to GLP-1 and Glucagon is shown below:

| | | | | | | | | | | | | | | | | | | | | | | | | | | | | | | | | SEQ |
|---|---|---|---|---|---|---|---|---|---|---|---|---|---|---|---|---|---|---|---|---|---|---|---|---|---|---|---|---|---|---|---|---|
| | | | | | | | | | | | | | | | | | | | | | | | | | | | | | | | | ID |
| Peptide | | | | | | | | | | | | | | | | | | | | | | | | | | | | | | | | No. |
| | 1 | | | | 5 | | | | | 10 | | | | | 15 | | | | | 20 | | | | | 25 | | | | | 30 | | |
| GLP-1 | H | A | E | G | T | F | T | S | D | V | S | S | Y | L | E | G | Q | A | A | K | E | F | I | A | W | L | V | K | G | R | G | 30 |
| Glucagon | H | S | Q | G | T | F | T | S | D | Y | S | K | Y | L | D | S | R | R | A | Q | D | F | V | Q | W | L | M | N | T | | | 31 |
| Analogues | H | Aib | Q | G | T | F | T | S | D | Y | S | K | Y | L | D | E* | Z1 | A | A | K* | E | F | I | Z2 | W | L | L | Q | T | NH₂ | | 33 |

Unnatural amino acids in the analogues are underlined and in italics; E* and K* indicate a side chain lactam linkage between Glu¹⁶ and Lys²⁰ for all analogs; and, Z1 and Z2 represent a Lys residue conjugated by acylation to various glycolipid surfactant-derived, duration of action modifiers (i.e., the surfactants discussed below). When either Z1 or Z2 is not present in the analogues, it is replaced by Q (Gln). The peptide analogues studied in this Example are shown in Table 14 below:

TABLE 14

| | | | | | | | | | | | | | | | | | | | | | | | | | | | | | | | | SEQ ID |
|---|---|---|---|---|---|---|---|---|---|---|---|---|---|---|---|---|---|---|---|---|---|---|---|---|---|---|---|---|---|---|---|---|
| cmpd # | 1 | | | | 5 | | | | | 10 | | | | | 15 | | | | | 20 | | | | | 25 | | | | | 30 | | NO. |
| Ref 27, sema | H | Aib | E | G | T | F | T | S | D | V | S | S | Y | L | E | G | Q | A | A | X | E | F | I | A | W | L | V | R | G | R | G | 11 |
| GLP-17-37 | H | A | E | G | T | F | T | S | D | V | S | S | Y | L | E | G | Q | A | A | X | E | F | I | A | W | L | V | K | G | R | G | 30 |
| Glucagon | H | S | Q | G | T | F | T | S | D | Y | S | K | Y | L | D | S | R | R | A | Q | D | F | V | Q | W | L | M | N | T | | | 31 |
| Ref 8, #32 | H | Aib | Q | G | T | F | T | S | D | Y | S | K | Y | L | D | E* | Q | A | A | K* | E | F | I | C | W | L | M | N | T | NH₂ | | 32 |
| 1 | H | Aib | Q | G | T | F | T | S | D | Y | S | K | Y | L | D | E* | Q | A | A | K* | E | F | I | Lys(GC8) | W | L | L | Q | T | NH₂ | | 12 |
| 2 | H | Aib | Q | G | T | F | T | S | D | Y | S | K | Y | L | D | E* | Q | A | A | K* | E | F | I | Lys(GC10) | W | L | L | Q | T | NH₂ | | 13 |
| 3 | H | Aib | Q | G | T | F | T | S | D | Y | S | K | Y | L | D | E* | Q | A | A | K* | E | F | I | Lys(GC12) | W | L | L | Q | T | NH₂ | | 14 |
| 4 | H | Aib | Q | G | T | F | T | S | D | Y | S | K | Y | L | D | E* | Q | A | A | K* | E | F | I | Lys(GC14) | W | L | L | Q | T | NH₂ | | 15 |
| 5 | H | Aib | Q | G | T | F | T | S | D | Y | S | K | Y | L | D | E* | Q | A | A | K* | E | F | I | Lys(GC16) | W | L | L | Q | T | NH₂ | | 16 |
| 6 | H | Aib | Q | G | T | F | T | S | D | Y | S | K | Y | L | D | E* | Q | A | A | K* | E | F | I | Lys(GC18) | W | L | L | Q | T | NH₂ | | 17 |
| 7 | H | Aib | Q | G | T | F | T | S | D | Y | S | K | Y | L | D | E* | Q | A | A | K* | E | F | I | Lys(MC12) | W | L | L | Q | T | NH₂ | | 18 |
| 8 | H | Aib | Q | G | T | F | T | S | D | Y | S | K | Y | L | D | E* | Q | A | A | K* | E | F | I | Lys(MeC12) | W | L | L | Q | T | NH₂ | | 19 |
| 9 | H | Aib | Q | G | T | F | T | S | D | Y | S | K | Y | L | D | E* | Q | A | A | K* | E | F | I | Lys(MeC14) | W | L | L | Q | T | NH₂ | | 20 |
| 10 | H | Aib | Q | G | T | F | T | S | D | Y | S | K | Y | L | D | E* | Q | A | A | K* | E | F | I | Lys(MeC16) | W | L | L | Q | T | NH₂ | | 21 |
| 11 | H | Aib | Q | G | T | F | T | S | D | Y | S | K | Y | L | D | E* | Q | A | A | K* | E | F | I | Lys(MeC18) | W | L | L | Q | T | NH₂ | | 22 |
| 12 | H | Aib | Q | G | T | F | T | S | D | Y | S | K | Y | L | D | E* | Lys(MeC14) | A | A | K* | E | F | I | Q | W | L | L | Q | T | NH₂ | | 23 |
| 13 | H | Aib | Q | G | T | F | T | S | D | Y | S | K | Y | L | D | E* | Q | A | A | K* | E | F | I | Lys(S₁GC14) | W | L | L | Q | T | NH₂ | | 24 |
| 14 | H | Aib | Q | G | T | F | T | S | D | Y | S | K | Y | L | D | E* | Q | A | A | K* | E | F | I | Lys(S₂GC14) | W | L | L | Q | T | NH₂ | | 25 |
| 15 | H | Aib | Q | G | T | F | T | S | D | Y | S | K | Y | L | D | E* | Q | A | A | K* | E | F | I | Lys(GC16c) | W | L | L | Q | T | NH₂ | | 26 |
| 16 | H | Aib | Q | G | T | F | T | S | D | Y | S | K | Y | L | D | E* | Q | A | A | K* | E | F | I | Lys(GC18c) | W | L | L | Q | T | NH₂ | | 27 |
| 17 | H | Aib | Q | G | T | F | T | S | D | Y | S | K | Y | L | D | E* | Lys(GC18c) | A | A | K* | E | F | I | Q | W | L | L | Q | T | NH₂ | | 1 |

Starred analogs have a Glu15 to Lys20 side chain lactam; G, M, Me in parentheses means D-glucoside, D-maltoside, D-melibioside linkages, respectively.
S₁ and S₂ mean a spacer of α-Lys or γ-Glu residue, respectively.
Cn means methylene chain of n carbons; c means carboxylate at end of chain.
X in semaglutide means a Lys residue acylated with γGlu-2xOEG (see ref 27) prolongation modifier comprising octadecandioic acid on a γGlu/short-PEG spacer.
Cmpd #33 in reference 8 refers to Cmpd #32 alkylated on Cys 24 with a 40 kDa PEG through a maleimide linker.

In Table 1, “Cmpd #” indicates analogues 1-17.

Structures of examples of glycolipid surfactant-based reagents used herein: 1-O-alkyl β-D-glucopyranosiduronic acid, 1′-O-alkyl [β-(α-D-galactopyranosiduronic acid-(1→6′)]-D-glucoside, or 1-O-alkyl β-[β-D-glucopyranosiduronic acid-(1→4)]-D-glucopyranosiduronic acid, respectively, are shown below:

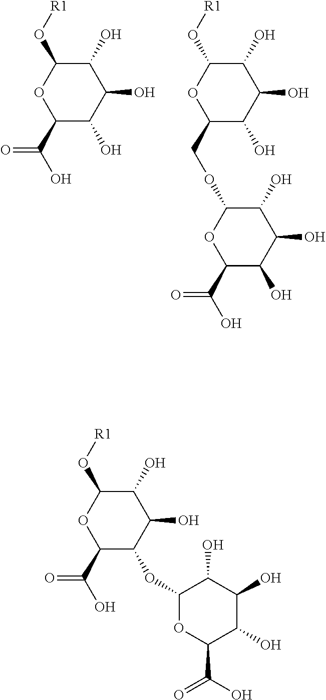

Reagents are prepared from the corresponding 1-O-alkyl β-D-glucoside, 1-O-alkyl β-D-melibioside, or 1-O-alkyl β-D-mannoside by chemoselective oxidation of the primary OH group(s). R1 alkyl groups may be straight, branched, saturated, unsaturated, normal or modified with functional groups. Physical properties and micellar character of surfactants are known to be dependent on specific head and tail group combinations. In this work, alkyl chain length of R₁ varies from C8 to C18. Linkage of the glycolipid modifier is through the 6- or 6′-(distal) carboxylic acid, typically by amide formation with the ε-amino function of a Lys residue in the peptide. For instance, such surfactant reagents are typically derived from commercially available non-ionic surfactants (Anatrace, Maumee, Ohio) by chemoselective oxidation of the primary alcohol group(s) on such surfactants using 2,2,6,6-tetramethylpiperidinyloxy (TEMPO)-mediated oxidation in the presence of water with [bis(acetoxy)-iodo]benzene (BAIB) as the oxidant at CS Bio Co (Menlo Park, Calif.). This reaction goes to completion with high chemoselectivity, producing virtually quantitative yield of the desired primary carboxylic acid with HOAc and Ph-I as the volatile, sole byproducts. Simple lyophilization of a pH 3 aqueous solution yields the desired free carboxylic acid ready for activation and coupling on a free amino group as the penultimate solid phase synthesis step prior to cleavage. Additional purification by trituration with Et₂O to remove traces of TEMPO can be applied, if desired, but is not necessary for the solid phase synthesis procedures used here. For larger scale oxidation, alternative stoichiometric oxidants (e.g. sodium hypochlorite) may be used. Coupling of the EuPort reagents proceeds more slowly than a normal amino acid coupling, typically requiring ≥8 hours to completion when at low molar excess. Additional glycolipid surfactants are prepared by Konigs-Knorr/Helferich glycosylation reaction on the appropriate alkyl alcohol and protected glycosyl bromide.

Solid phase peptide synthesis used in producing the peptide analogues of this Example used standard Na-Fmoc protocols (t-butyloxycarbonyl and N-trityl side chain protection; plus Arg(Pbf); Nα-Boc-His(Trt)) on Rink amide resin at CS Bio Co (Menlo Park, Calif.), orthogonal protection on the Glu¹⁶ and Lys' positions (allyl ester and Nε-allyloxycarbonyl, respectively). The Lys position to be modified by EuPort conjugation was protected with Nε-1-(4,4-dimethyl-2,6-dioxocyclohex-1-ylidene)-3-methylbutyl (iv-Dde), selectively deprotected as the penultimate step with 4% hydrazine in DMF and coupled with the appropriate EuPort reagent (as carboxylic acid) using DIC and HBT (or other coupling additive, as desired). Final peptides were cleaved and deprotected using trifluoroacetic acid (TFA)/water/triisopropylsilane (95:2.5:2.5), precipitated with ether, washed with ether, dried and purified by appropriate reversed-phase (C-18) HPLC chromatography using acetonitrile in TFA (0.1%) buffer gradients. Compounds were characterized by analytical HPLC/mass spectrometry using similar buffers on analytical columns and all tested analogs had purity ≥95% (Table 15).

TABLE 15

| | | Expected | Actual | | ^{[b]}HPLC | |
|---|---|---|---|---|---|---|
| Analogue # | | Molecular | Molecular | ^{[a]}Purity | retention | |
| (SEQ ID NO.) | | Weight (Da) | Weight (Da) | (%) | k′ (method) | |
| 1 | (12) | 3703.15 | 3702.42 | 98 | 3.6 | (a) |
| 2 | (13) | 3731.15 | 3731.42 | 99 | 2.7 | (b) |
| 3 | (14) | 3759.22 | 3769.04 | 96 | 2.6 | (c) |
| 4 | (15) | 3787.22 | 3786.88 | 95 | 2.8 | (c) |
| 5 | (16) | 3815.22 | 3815.52 | 96 | 4.8 | (d) |
| 6 | (17) | 3843.22 | 3843.12 | 96 | 3.9 | (d) |
| 7 | (18) | 3935.35 | 3934.66 | 96 | 4.1 | (b) |
| 8 | (19) | 3921.35 | 3921.15 | 95 | 3.4 | (b) |
| 9 | (20) | 3949.42 | 3950.22 | 98 | 15 | (d) |
| 10 | (21) | 3977.47 | 3976.84 | 99 | 3.0 | (d) |
| 11 | (22) | 4005.55 | 4004.64 | 96 | 4.1 | (d) |
| 12 | (23) | 3977.47 | 3977.67 | 95 | 3.8 | (c) |
| 13 | (24) | 3915.39 | 3915.72 | 99 | 3.9 | (b) |
| 14 | (25) | 3916.33 | 3916.98 | 95 | 4.2 | (e) |
| 15 | (26) | 3845.28 | 3845.16 | 95 | 2.9 | (b) |
| 16 | (27) | 3873.34 | 3873.46 | 95 | 5.9 | (b)* |
| 17 | (1) | 3873.34 | 3872.94 | 95 | 3.4 | (b) |

^{[a]}Purity is estimated by integration of the post injection-anomaly peaks.
^{[b]}The k′ is an HPLC system-independent measure of retention, k′ = (tᵣ − t₀)/t₀. Analyses were run on Phenomenex Luna 5μ C-18 250 × 4.6 mm columns at 1 mL/min;
*16 was similarly run on a Polymer Labs PLRP-S 100A 8μ 250 × 4.6 mm column. Elution gradients are from low % B to high % B (B is % CH₃CN in 0.1% TFA) in min: a = 35 to 65% in 20; b = 40 to 70% in 20; c = 45 to 75% in 20; d = 50 to 80 in 20; e = 30 to 90 in 20.

A. In Vitro Receptor activation Assay

Receptor activation assays were performed at DiscoverX laboratories (Fremont, Calif.) using human GLP-1R and GCGR cloned into Chinese Hamster Ovary (CHO) cells (LeadHunter Discovery Services; assay product 86-0007D cAMP Hunter™ using huGLP1R and huGCGR; whole cell cAMP accumulation assays; cell lines used were cAMP Hunter™ CHO-K1 GCGR Gs Cell Line, catalog 95-0042C2 and cAMP Hunter™ CHO-K1 GLP1 Gs Cell Line, catalog 95-0062C2; readout of accumulated cAMP was made using readout HitHunter cAMP XS+ assay). Cell lines are maintained at DiscoverX and were incubated with test agents for 30 m at 37° C. for accumulation of cAMP. Results are evaluated at DiscoverX using in house parameters and the performance of literature standards (exendin-4 and glucagon, for GLP-1R and GCGR respectively) in parallel in order to be reportable. Results described herein are from single assays performed on cells in duplicate and data were replotted in Prism 5 to provide pEC₅₀ (SE) data. There were cytotoxicity observations reported for any of the assays. Most assays were carried out in the presence of 0.1% BSA to minimize non-specific binding, but assays on 15-17 were also tested in the presence of 0.1% chicken ovalbumin (OVA). For these compounds, which bind very tightly to BSA (>99%; data not shown), its presence can distort the results to make the compounds seem much less potent.

B. In Vitro Stability Plasma

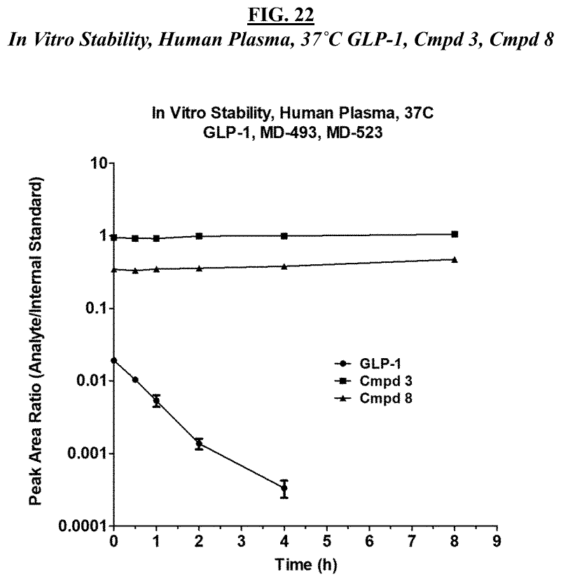

The stability test was carried out at Climax Laboratories, Inc. (San Jose, Calif.). Samples of the test articles (circa 0.5 mg, GLP-1 7-36 amide, Bachem; analog 3; analog 5) were dissolved in pooled human plasma (Bioreclamation LLC, lot—BRH 392992) at a concentration of 1 to 10 μM and compound levels remaining at time points given were quantitated as described in Bioanalytical Method (2.54). The time/concentration course (FIG. 22) indicated that GLP-1 7-36 amide was rapidly destroyed to below quantitation limit (BQL; circa 2 ng/mL) after 4 h of incubation while the amounts of analogs 3 and 5 were unchanged at 8 h, indicating their excellent intact stability in the presence of pooled human plasma.

The stability of analogue 17 (SEQ ID NO: 1, as in ALT-801) in plasma, specifically its binding to the plasma protein albumin, was also studied. Such non-covalent binding to albumin is anticipated to slow down the degradation of peptide in plasma and results in decreased renal clearance. Binding of ALT-801 (15000 ng/mL) to plasma proteins of rat, dog, monkey, and human was assessed by ultracentrifugation for six hours. Pooled plasma was obtained from at least three Sprague Dawley rats, beagle dogs, and cynomolgus monkeys. Pooled human plasma was obtained from three human males (that reportedly had not taken any medication in the previous 7 days before collection). K₂EDTA was used as the anticoagulant. The pH of each pool of plasma was adjusted, if needed, to approximately pH 7.4 with hydrochloric acid or sodium hydroxide. Ultracentrifugation was performed using polycarbonate ultracentrifugation tube placed in a S80 AT2 rotor at 37° C. at 357000×g for 6 hours to achieve separation of PUC (supernatant) from plasma proteins. After centrifugation, the PUC was analyzed by LC MS for the calculation of protein binding. Protein Binding was evaluated as Percent unbound=(Cu/Co)×100 and Percent bound=100-percent unbound where Co is the concentration of test article in plasma prior to ultracentrifugation (ng/mL) and Cu is the concentration of test article in plasma in the ultracentrifugate (ng/mL). The results are presented in Table 16.

TABLE 16
Percentages of bound and unbound ALT-801 (15000 ng/mL) in rat, dog, monkey,
and human plasma after ultracentrifugation at 37° C. for 6 hours

| | Initial Conc | | | Ultracentrifugate | | | | | | | |
|---|---|---|---|---|---|---|---|---|---|---|---|
| | (ng/mL) | | Percent | (ng/mL) | | | Percentage of ALT-801 | | | | |
| Species | Rep | Mean | of Theoretical | Rep | Mean | SD | Unbound | Mean | Bound | Mean | SD^{a} |
| Rat | 6000 | 5980 | 39.9 | 11.0 | 14.4 | 4.89 | 0.184 | 0.241 | 99.8 | 99.8 | 0.0817 |
| | 5960 | | | 20.0 | | | 0.334 | | 99.7 | | |
| | 6200 | | | 12.2 | | | 0.204 | | 99.8 | | |
| Dog | 5950 | 6070 | 40.4 | 16.1 | 12.4 | 4.34 | 0.265 | 0.205 | 99.7 | 99.8 | 0.0716 |
| | 6180 | | | 13.5 | | | 0.223 | | 99.8 | | |
| | 7270 | | | 7.62^{b} | | | 0.126 | | 99.9 | | |
| Monkey | 15600 | 14500 | 96.4 | 7.39 | 7.05 | 0.257 | 0.0511 | 0.0487 | 100 | 100 | 0.00178 |
| | 14500 | | | 6.86 | | | 0.0474 | | 100 | | |
| | 13300 | | | 7.10 | | | 0.0491 | | 100 | | |
| | | | | 6.84 | | | 0.0473 | | 100 | | |
| Human | 7580 | 7610 | 50.7 | BLQ | 16.2 | NA | NA | 0.213 | NA | 99.8 | NA |
| | 7630 | | | 8.87^{b} | | | 0.117 | | 99.9 | | |
| | 7780 | | | 23.5 | | | 0.309 | | 99.7 | | |

BLQ Below the limit of quantitation.
Conc Concentration.
Rep Replicate.
SD Standard deviation.
^{a}Standard deviation applies to both bound and unbound percentages.
^{b}Value BLQ, extrapolated value shown.

The mean percent protein binding of ALT-801 was 99.8% in rat plasma, 99.8% in dog plasma, 100% in monkey plasma, and 99.8% in human plasma. These results indicated that ALT-801 has extensive protein binding (≥99.8%) in plasma of rat, dog, monkey, and human.

C. Pharmacokinetics

PK and PD assays were carried out following standard protocols in rats at Charles River Laboratories (Shrewsbury, Mass.), and in db/db mice at JAX Laboratories (Sacramento, Calif.). PK studies were also carried out in Gottingen mini pigs at MPI Research (Mattawan, Mich.) or Yucatan mini-swine. There were no observations of compound-related injection site reactions for any compounds tested. Bioanalytical analysis by LC/MS/MS was carried out at Climax Laboratories, Inc. (San Jose, Calif.) or, for the Yucatan mini-swine study, at Frontage Laboratories, Inc. (Exton, Pa.).

D. Pharmacokinetics in Rats

The PK behavior of 17 (as ALT-801) and of semaglutide, following a single sc dose of 10 nmol/kg were evaluated at Charles River Laboratories in male CRL:CD(SD) rats (250-300 g). Both ALT-801 and semaglutide were formulated at 0.1 mg/mL in 50 mM phosphate buffer containing 0.05% tween 80 at pH ˜8. Blood samples (˜300 μL) were collected at 2, 4, 8, 24, 48, 72, 96, 120 and 144 h post-dose (n=4 per time point) into ice cooled K₂EDTA tubes and stored on ice until processing to plasma by centrifugation at 2200 rpm for 10 min at 5° C. Plasma conc of ALT-801 and semaglutide were determined as outlined in Bioanalytical Method (2.5.3) below.

1. Pharmacokinetics in Göttingen Minipigs

This study uses a cassette style dosing, to minimize large animal usage, but with injection subcutaneously at separate sites to preclude each compound's influencing uptake of another compound. A total of two male Gottingen minipigs were assigned to study. The animals were pair housed in pens on raised floor caging. The animals weighed between approximately 11-15 kg at transfer and approximately 5-8 months of age. The same animals were to be used for multiple phases, following a minimum 1-week washout period. To facilitate dosing and to ensure animal safety during the dosing procedure, animals were sedated with Telazol (IM, 4-6 mg/kg) prior to dosing. Dosing was subcutaneous via bolus injection between the skin and underlying layers of tissue in the ventral region of the animal. A total of 3-4 sites were used for each phase with different compounds dosed at each of the 4 sites. Compounds are formulated in saline containing 0.2% BSA (circa 0.4 mg/mL) at pH 3.5. Each stock solution was diluted with normal saline (pH 7.4) to the required final conc and sterile filtered. Dosing is at 20 nmol/kg. Blood samples were collected pre-dose and at 2, 4, 6, 8, 12, 24, 36, 48, 72 and 96-hours post-dose. At each blood collection time point a 1 mL sample is taken from the jugular vein into K₂EDTA tubes on ice before processing to plasma by centrifugation. The plasma samples containing the 4 test compounds were sent to Climax Labs for separation and quantitation by LC-MS/MS as noted below (2.5.3).

2. Pharmacokinetics in Yucatan Mini-Swine

The test animals were a total of four non-naïve male Yucatan mini-swine (Sus scrofa; body weight 73-81 kg), housed singly. Animals were fed a maintenance amount of Purina S-9 swine diet. General, in-cage observations were made at least twice daily (morning and evening) during the study period to assess general health, moribundity or mortality.

Following an acclimation period of twenty-two days each mini-swine was dosed subcutaneously (behind cheek jowl) with 17 at 20 nmol/kg (0.2 mL/kg), and PK blood samples were collected at −0.25, 2, 4, 6, 8, 12, 24, 48, 72, 96, 120, 168, 192, 216, 264, 312 and 360 h post-dose. Following a two-week washout period, the same animals were administered 17 iv and PK blood samples were collected at −0.25, 0.25, 0.5, 1, 2, 4, 8, 12, 24, 48, 72, 96, 120, 168, 192, 216, 264, 312 and 360 hours post-dose. Dose concentration was 5.5 mg/mL (dose volume 0.015 mL/kg) for both treatments. Whole blood samples for pharmacokinetic analysis (˜3 mL/time point) were collected via vascular access ports into tubes containing K₂EDTA. Samples were maintained on wet ice until processing, ˜30 minutes or less post-collection. All samples were centrifuged for ˜15 minutes at 3000 rpm and ˜4° C. Plasma samples were stored frozen at −70° C., until primary samples were shipped to Frontage Laboratories (Exton, Pa.) for bioanalysis by LC-MS/MS similarly to as outlined below. No abnormal clinical observations were noted during study conduct.

E. Pharmacodynamics.

1. Effect on Blood Glucose—db/db Mice

About seventy-five (75) BKS·Cg-m+/+Leprdb/J (Jackson Labs stock number 000642) male (“db/db”) mice at the age of 7-9 weeks of age were used in these studies and maintained using standard animal care procedures. Studies initiated after one-week acclimation to facility conditions. On the morning of study day 0, mice were weighed and fasted for 4 h Blood glucose was measured by glucometer using standard procedures. At least fifty-four (54) mice were selected based on body wt and those with blood glucose levels >300 mg/dL (i.e., diabetic) were randomly assigned into 6 groups (n=9). Groups were as follows: group 1, vehicle; group 2, semaglutide 3 nmol/kg; group 3, semaglutide 10 nmol/kg; group 4, 17, 1 nmol/kg; group 5, 17, 3 nmol/kg; group 6, 17, 10 nmol/kg. Body weights were measured and recorded at receipt, prior to randomization, and daily from Days 1 to 5. Food consumption was measured and recorded daily from Days 1 to 5. Blood samples for glucose analysis were collected pre-test (Day −3) and at 0, 2, 4, 8, 24, 48, 72, 96 and 120 hours following the single dose of the indicated compound.

2. Body Weight—“DIO CRL:CD(SD)” Rats.

Fifty-four male DIO CRL:CD rats, approximately 14-15 weeks of age upon study initiation, were enrolled in the study at Charles River Laboratories (Shrewsbury, Mass.). Animals were maintained on a high fat diet (Research Diets 12492, 60% kcal % fat) for a period of 11 weeks prior to arrival at the testing facility. Upon arrival, animals were maintained on high fat diet for a period of 7 d during acclimation, and throughout the duration of the study. Food consumption was monitored on Study Days −1 through Study Days 27 (Main Study) or Day 41 (Recovery) by weighing the food and hopper together. The mean value of food consumption for Group 2 determined the amount of food made available to Group 3 in the subsequent feeding session. Similarly, the mean value of food consumed for Group 5 determined the quantity of food available for Group 6 in the subsequent feeding session. Food and drinking water were provided ad libitum throughout the study with the exception of the 5-h fasting periods occurring on Study Days 1, 28, and 42. Animals were randomized into groups based on body weight and non-fasted blood glucose (BG) data collected on Study Day −1. On Study Days 1 through 27 (Main Study) or 42 (Recovery) all animals were administered a bolus dose of vehicle, semaglutide standard (12 nmol/kg), or 17 (6, 12 nmol/kg) via sc interscapular injection. The total group dependent dose volume (mL/kg) was based on the most recently recorded body wt. Individual animal body weights were recorded beginning on Day −1. Throughout dosing and at all sample collection time points, the animals were observed for any clinically relevant abnormalities. On Study Days −1, 1, 3-27, 29, and 36 a 3 μL drop of whole blood was collected via tail snip for assessment of blood glucose using a handheld glucometer (Alpha Trak 2, Abbot). With the exception of Day 1 during which blood glucose readings were taken pre-dose, 2, 4, 8, and 24 hours post dose, readings were taken at approximately the same time daily. Additionally, on Study Day 28 following a 5-hour fast, animals were administered a 10 mL/kg dose of glucose (2 g/kg) via intraperitoneal injection. A 3 μL sample of blood was collected via tail snip and analyzed for glucose levels at the following timepoints (relative to glucose administration): 0, 15, 30, 60, 90, 120, and 180 minutes post dose. Samples for glucose were read using a handheld glucometer.

F. Bioanalytical Method

Analysis was carried out at Climax Laboratories (San Jose, Calif.) on an API-4000 Mass Spectrometer, ESI positive, MRM scan. Samples were loaded on a Shimadzu HPLC/CTC Autosampler with an ACE C4 column (2.1×50 mm, 5 μm). Elution was by gradient from aqueous 0.5% formic acid, 5 mM NH₄OAc to 0.5% formic acid in CH₃CN/H₂O (9:1). Plasma samples (100 μL) were plated (96 well) and 30 μL of internal peptide standard was added (10 m/mL in PBS). A 300 μL aliquot of CH3CN was added and the sample was vortexed and centrifuged to precipitate plasma proteins. After transfer to a 96 well plate, a 40 μL sample was injected and the individual compound peaks were quantitated with standard curves. Non-compartmental pharmacokinetic analysis using WinNonlin was performed by using the mean conc at each sampling time point to report the maximum conc (Cₘₐₓ), the time Cₘₐₓ was observed (Tₘₐₓ), the area under the plasma conc curve from time zero to the last time point with measurable conc (AUC₀₋₄), the plasma conc-time curve from time zero to infinity (AUC_{0-∞}), the terminal elimination half-life (t_{1/2}), and the MRT. The quantitation limit is 1-2 ng/mL, depending on analog structure.

G. Statistical Analysis

In vitro data are presented as pEC₅₀ (SE) determined in Prism 5 by nonlinear regression analysis of the raw fluorescence data normalized by corresponding response to internal standards (see Supporting Information for data plots). For assays where statistical significance is cited GraphPad Prism software (version 5) was used to conduct the statistical data analysis, performing Analysis of Variance (ANOVA type 2 with multiple measures) followed by Bonferroni tests with p<0.05 as the minimal level of significance.

H. Peptide Prolongation

Our approach toward increasing the serum half-life of peptide GLP-1R/GCGR dual agonists focused on a new approach, the use of covalently linked glycolipid surfactant-derived modifiers. The reagents were derived predominantly from those of commercial type, non-ionic surfactants that are used widely in the cosmetic and pharmaceutical industries and are Generally Recognized As Safe, e.g., 1-octyl β-D-glucose and 1-dodecyl β-D-maltose (Anatrace, Maumee Ohio). Additional surfactant structures are available by Koenigs-Knorr/Helferich glycosylation (e.g. HgO (yellow)/HgBr₂ catalysis) of acetobrom glucose (or similar activated carbohydrates) with the appropriate alcohol and deprotection with NaOMe/MeOH to yield the free surfactant. The desired reagents are readily available through a chemoselective TEMPO-mediated oxidation, in the presence of water, of the primary alcohol group(s) on such surfactants. The typical structure therefore comprises 1-O-alkyl β-D-glucopyranosiduronic acids (also known as 1-O-alkyl β-D-glucuronic acid adducts), a type of structure frequently formed in the liver (Phase II metabolism) for solubilization/detoxification of hydrophobic molecules, here acylated to a Lys residue. Solid phase peptide synthesis of the desired peptides used standard Fmoc protocols with orthogonal protection on the Glu¹⁶ and Lys²⁰ positions (allyl ester and Alloc, respectively) used to allow side chain lactam formation and N-ε-ivDde on the Lys position to be modified by glycolipid surfactant conjugation. The peptides were obtained in high purity (>95%, analytical rp-hplc) and with good yields.

I. Pharmacokinetic Behavior

A major objective of these studies was the investigation of the effects of the novel, glycolipid surfactant-conjugation approach for increased duration of action, stability, potency and bioavailability for a peptide. A preliminary in vitro stability study in pooled human plasma demonstrated the rapid destruction of GLP-1 7-36 amide (at 4 hr), while concentrations of analogs 3 and 8 were completely unchanged at 8 h, indicating excellent stability for these representative surfactant-conjugated analogs in the presence of plasma.

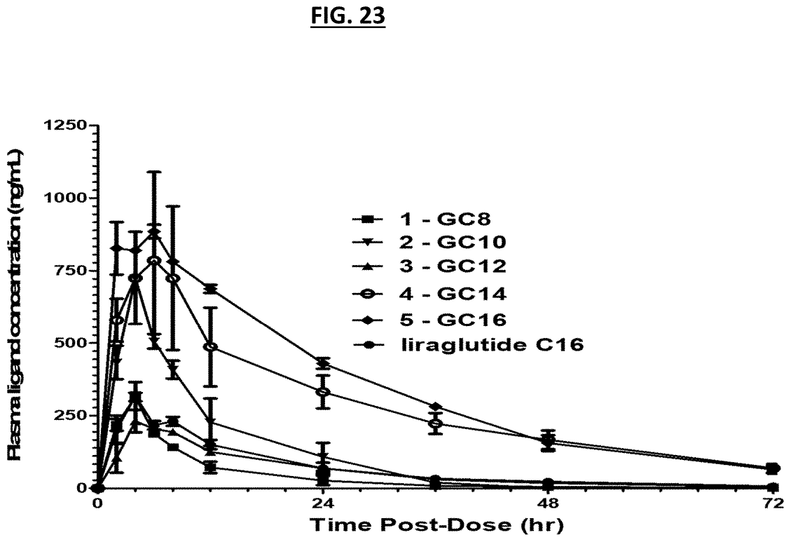

The duration of action of analogs was evaluated in rodent and mini-pig models. The compound series (analogues) 1 through 6 (Table 15) was designed to examine the effect on potency and duration of action for a homologous increase in length and hydrophobicity (from octyl to hexadecyl) of the alkyl chain in position 1 of the 1-O-alkyl β-D-glucopyranosiduronic acid modifier. As seen in FIG. 23, the relationship between chain length and duration of action in a PK study in Gottingen minipigs was not strictly proportional. One can envision multiple variables potentially affecting the measured PK and PD as chain length increases. For example, for chain length increase: depot formation (increase), solubility (decrease), affinity for SA (increase), hormone receptor affinity (increase then decrease), potency for receptor activation (increase then decrease). For this group, the Cₘₐₓ and PK profiles appear optimal for 4 (C14) and 5 (C16), possibly due to optimal solubility and SA association yielding good distribution. The behavior of liraglutide as a standard in this assay (acylated with palmitic acid, C16, on a γGlu spacer) most closely matched that of analogue 3 (C12), containing a shorter side chain. In vivo pharmacokinetic behavior of compounds following subcutaneous administration to Gottingen minipigs at 20 nmol/kg for each analogue. All data from a single assay except for 4 and 5, which were from parallel assays in Gottingen minipigs, also profiled against liraglutide as literature standard. Significantly higher plasma levels are measured for analogue 2 at 4 h (**); for analogue 4 at 2 and 4 h (**), 6 and 8 h (***) and 12 h (*); for analogue 5 at 2 and 12 h (***), and at 24 h (*), all compared to liraglutide: *, P<0.05; ** P<0.01; *** P<0.001.

J. In Vitro Structure Activity Analysis

We sought highly potent analogues with evenly balanced agonistic activity at both GLP-1R and GCGR, coupled with good in vivo bioavailability and very prolonged duration of action. Another goal was the understanding of the effects of the novel glycolipid surfactant modification on potency and duration of action. Accordingly, the peptide structure is identical for most of the analogs studied. Initial SAR studies were directed at evaluation of the analogues' potency for activation of the cloned human receptors in vitro (Table 17). EC₅₀ values for compounds 1-4 (side chain modifications from 1-β-octyl β-D-glucopyranosiduronyl to 1-O-tetradecyl β-D-glucopyranosiduronyl) showed highly potent and variably balanced activation, with EC50 values in the 10-30 pM range and a Selectivity Ratio (SR=GCGR EC50/GLP-1 EC₅₀) from 2 to 3. The GCGR appears to be more sensitive to steric effects in that 5 to 6 (C16, C18) show rapidly elevating EC₅₀ values for it (163 to 884 pM), with an increasing bias toward GLP-1R activation (SR=4× and 17×, respectively). Detailed optimization of the assay for such hydrophobic analogs was not carried out but the EC₅₀ values for the GLP-1R did not rise as rapidly.

TABLE 17
Analogue structures and in vitro evaluation of biological
activity on relevant cloned human receptors

| Analog | | Surfactant | pEC₅₀ (SE)^{[b]} | | EC50 (pM) | | Selectivity Ratio^{[c]} |
|---|---|---|---|---|---|---|---|
| (SEQ ID No.) | | Modification^{[a]} | huGCGR | hu GLP-1 | hGCGR | hGLP-1R | GLP-1R vs GCGR |
| 1 | (12) | Lys²⁴(GC8) | 10.70 (0.03) | 11.07 (0.03) | 20 | 8 | 3 |
| 2 | (13) | Lys²⁴(GC10) | 10.70 (0.03) | 10.90 (0.03) | 20 | 13 | 2 |
| 3 | (14) | Lys²⁴(GC12) | 10.54 (0.02) | 10.74 (0.03) | 29 | 18 | 2 |
| 4 | (15) | Lys²⁴(GC14) | 10.52 (0.05) | 10.80 (0.03) | 30 | 16 | 2 |
| 5 | (16) | Lys²⁴(GC16) | 9.79 (0.03) | 10.40 (0.04) | 163 | 40 | 4 |
| 6 | (17) | Lys²⁴(GC18) | 9.05 (0.02) | 10.28 (0.02) | 884 | 52 | 17 |
| 7 | (18) | Lys²⁴(MC12) | 10.83 (0.12) | 10.30 (0.02) | 15 | 50 | 0.3 |
| 8 | (19) | Lys²⁴(MeC12) | 10.42 (0.02) | 10.49 (0.03) | 32 | 32 | 1 |
| 9 | (20) | Lys²⁴(MeC14) | 10.45 (0.01) | 11.31 (0.04) | 35 | 5 | 7 |
| 10 | (21) | Lys²⁴(MeC16) | 9.65 (0.03) | 11.10 (0.02) | 225 | 8 | 28 |
| 11 | (22) | Lys²⁴(MeC18) | 9.31 (0.02) | 10.47 (0.04) | 486 | 34 | 14 |
| 12 | (23) | Lys¹⁷(MeC14) | 10.35 (0.02) | 10.64 (0.03) | 45 | 23 | 2 |
| 13 | (24) | Lys²⁴(S1GC14) | 9.76 (0.02) | 10.43 (0.02) | 174 | 37 | 5 |
| 14 | (25) | Lys²⁴(S₂GC14) | 10.01 (0.02) | 10.37 (0.05) | 97 | 43 | 2 |
| 15 | (26) | Lys²⁴(GC16c) | 10.19 (0.02) | 10.42 (0.02) | 65^{[d]} | 38^{[d]} | 2 |
| 16 | (27) | Lys²⁴(GC18c) | 10.06 (0.02) | 10.36 (0.02) | 86^{[d]} | 44^{[d]} | 2 |
| 17 | (1) | Lys¹⁷(GC18c) | 10.38 (0.02) | 10.41 (0.02) | 42^{[d]} | 39^{[d]} | 1 |

^{[a]}All structures have Glu¹⁶ to Lys²⁰ side chain lactam; G, M, Me means D-glucoside, D-maltoside, D-melibioside linkages, respectively. S1 and S2 mean a spacer of α-Lys or γ-Glu residue, respectively, between Lys and surfactant. Cn means methylene chain of n carbons; c means carboxylate at end of chain.
^{[b]}All screening data generated at DiscoverX from accumulated cAMP response in CHO cells (in duplicate) expressing hCCGR and hGLP-1R, using non-linear regression analysis with an R² typically >>90%. Data were replotted and analyzed in Prism 5 to report pEC₅₀ (SE) values and curves are displayed in Supporting Information.
^{[c]}Selectivity Ratio generated from EC₅₀ data in pM (SR = GCGR EC₅₀/GLP-1 EC₅₀).
^{[d]}Data for compounds 15, 16, 17 were obtained in the presence of 0.1% OVA containing buffers. For all others, 0.1% BSA containing buffers were used.

The physical properties of such surfactant-modified peptides can be expected to be widely varied and tunable by the use of various alkyl chains (varied hydrophobicity, solubility, SA affinity, CMC, micelle size) and also by use of different carbohydrate head groups, such as disaccharides (varied solubility, micelle size, Hydrophile-Lipophile Balance), in the glycolipid surfactant precursors. Thus “dodecyl maltoside” is a widely used commercial surfactant and its use here yields 7, a highly potent but GCGR favoring dual agonist. This surfactant is less convenient than glucose in that it has two primary OH groups and therefore yields two carboxyl functions upon oxidation, albeit one more sterically hindered than the other.

As a disaccharide head group, melibiose is more useful, with only one glycosylation site and a single primary OH function for oxidation to uronic acid. Use of melibiose yields 1′-O-alkyl [β-(α-D-galactopyranosiduronic acid-(1→6′))]-D-glucoside intermediates (MeCl2-MeCl8) and results in analogues 8-12. This disaccharide series contains very potent (7) and well-balanced (8) dual agonists, while also showing evidence suggesting steric hindrance to activation of the GCGR (9-11).

While the 1-O-dodecyl β-D-maltoside-derived modification favored GCGR activation (7; SR 0.3), the 1-dodecyl β-D-melibioside-derived analog, 8, had nearly balanced selectivity receptor potency (SR ˜1). Further increases in the size of the melibioside-based modification (C14, C16, C18; 9-11) rapidly decreased the GCGR potency (SR of 7, 28, 14, respectively). Increased ligand side chain size (or hydrophobicity) again appears to disfavor GCGR activation.

All the forgoing modifications were placed at residue 24, toward the C-terminal side of the side chain lactam linkage (Glu¹⁶ to Lys²⁰). Side chain modification within the lactam ring was also studied by placement of a Lys(Me14) residue at position 17 (compound 12) and again found high potency with only a modest bias toward GLP-1R activation (SR 2). In contrast the same modification in position 24 had shown strong bias toward GLP-1R (SR 7). Perhaps the conformation in the region of attachment for 12, within the lactam ring, is disfavoring the GLP-1R activation for this combination of head group and chain length.

Mid-length glycolipid surfactant modifications yielded highly potent and relatively balanced analogs so we next examined the effect of a spacer linkage to the hydrophobic side chain modification, as seen with liraglutide, semaglutide, and other similar compounds. Such linker attachment was found to be critical for potency in the semaglutide drug design story, with 15 linkers being investigated with wide variations in potency before settling on a γGlu-short PEG sequence linker. Thus compound 14 has a Glu(γCO) linked to the Lys²⁴ position with a 1-O-tetradecyl β-D-glucopyranosiduronyl modification linked to the Glu(α-NH₂) function (S₂GC14), and this modification has significantly weakened GCGR activation potency (vs 4). Use of a Lys(α-CO) linkage to the Lys²⁴ as spacer and linkage of the 1-O-tetradecyl β-D-glucopyranosiduronyl modification to the spacer's ε-amino function yielded 13, a molecule very unfavorable for GCGR interaction (SR 5). In addition to added bulk, the Glu(γCO) linker adds a negative charge to the linkage position while the Lys(α-CO) linkage adds a positive charge to this side chain linker. Importantly, our glycolipid surfactant modification appears not to require any spacers or spacer-receptor interactions, as seen for other side chain modifiers, in order to yield highly potent molecules.

While we previously studied primarily substitution of peptide sequences with hydrophobic amino acids as a route to high HSA binding, here structures 15-17 are analogues designed to test the effect of mimicking the head group of fatty acids by incorporation of a carboxylic acid function at the terminus of the surfactant alkyl chain, similar to that used in semaglutide. Accordingly, 15 incorporates 1-O-[(15-carboxypentadecyl)oxy] β-D-glucopyranosiduronic acid in amide linkage to the ε-NH group on Lys²⁴ (Lys²⁴GC16c) while 16 contains 1-O-[(17-carboxyheptadecyl)oxy] β-D-glucopyranosiduronic acid similarly attached on Lys²⁴ (Lys²⁴GC18c). Similarly, 17 contains 1-O-[(17-carboxyheptadecyl)oxy] β-D-glucopyranosiduronic acid, but the glycolipid surfactant conjugation is to Lys¹⁷ (Lys¹⁷GC18c), as for 12, thus linked within the lactam ring formed between the side chains of Glu¹⁶ and Lys²⁰. Analogue 17 exhibited high potency, strong evidence for very high serum albumin (SA) binding and evenly balanced dual receptor activation potency (SR=˜1; Table 16). Accordingly, analogue 17 was selected for more detailed characterization studies.

It is well known that strong SA binding can result in diminished potency in vitro and in vivo, and this is documented with respect to the binding of semaglutide to GLP-1R, wherein the ratio of binding in the presence of 2% HSA resulted in a remarkable 940× decrease in measured affinity as compared to binding in the absence of HSA. Nonetheless manipulation of peptides in solution without the presence of some protein to block non-specific binding also can cause decreased apparent potency through loss of ligand. Use of OVA, which has not evolved to be a fatty acid carrier protein and has minimal fatty acid binding properties, is a useful alternative. A comparison of EC₅₀ data for activation of human GLP-1R and GCGR cloned into Chinese Hamster Ovary (CHO) cells (DiscoverX) by compounds 15-17 and semaglutide. The ratio of EC₅₀ measured in the presence of BSA vs OVA can be taken as a qualitative measure of the BSA affinity. Here one can see that the improvement on replacing even a low conc of BSA (0.1%) with OVA (0.1%) was negligible for the assay standards exendin-4 and glucagon, while the effect for the C16 side chain of analogue 15 was modest (fold improvement of 4-9×). In contrast, for 16 or 17, which have C18 alkyl chains, the effect of replacement of BSA by OVA was profound (fold improvement of 29-47×). The improvement for 16 and 17 is even greater than that seen for semaglutide (13×), suggesting that one may expect to see even tighter-binding and even longer duration of action for 17 than for semaglutide. This data is presented in Table 18.

TABLE 18
Benefit of replacing BSA by OVA in
receptor activation assay buffer

| Analogue | |
|---|---|
| pEC₅₀ (SE) | pEC₅₀ (SE) |
| 0.1% BSA | 0.1% OVA |

| Receptor | GLP-1 | GCG | GLP-1 | GCG |
|---|---|---|---|---|
| 15 | 9.71 | 9.25 | 10.42 | 10.19 |
| | (0.05) | (0.15) | (0.02) | (0.02) |
| 16 | 8.90 | 8.53 | 10.36 | 10.06 |
| | (0.06) | (0.08) | (0.02) | (0.02) |
| 17 | 8.95 | 8.71 | 10.41 | 10.38 |
| | (0.04) | (0.12) | (0.02) | (0.02) |
| semaglutide | 9.72 | N/A | 10.82 | N/A |
| | (0.09) | | (0.03) | |

| | Analogue | | | | | |
|---|---|---|---|---|---|---|
| | EC₅₀ (pM) | | EC₅₀ (pM) | | Fold | |
| | 0.1% BSA | | 0.1% OVA | | Improvement^{[a]} | |
| Receptor | GLP-1R | GCGR | GLP-1R | GCGR | GLP-1R | GCGR |
| Glucagon | | 49 | | 68 | | 0.7 |
| Exendin-4 | 18 | | 15 | | 1 | |
| 15 | 194 | 564 | 38 | 65 | 5 | 9 |
| 16 | 1,268 | 2929 | 44 | 86 | 29 | 34 |
| 17 | 1,117 | 1957 | 39 | 42 | 29 | 47 |
| semaglutide | 192 | N/A | 15 | | 13 | |

^{[a]}Fold Improvement = (EC₅₀ in presence of BSA/EC₅₀ in presence of OVA) and is hypothesized to indicate degree of binding to BSA, since its replacement with non-binding OVA increases observed potency (lowered EC₅₀). As discussed herein, exceptionally tight BSA binding distorts the actual receptor activation potency for semaglutide and these analogues.

K. In Vivo Characterization

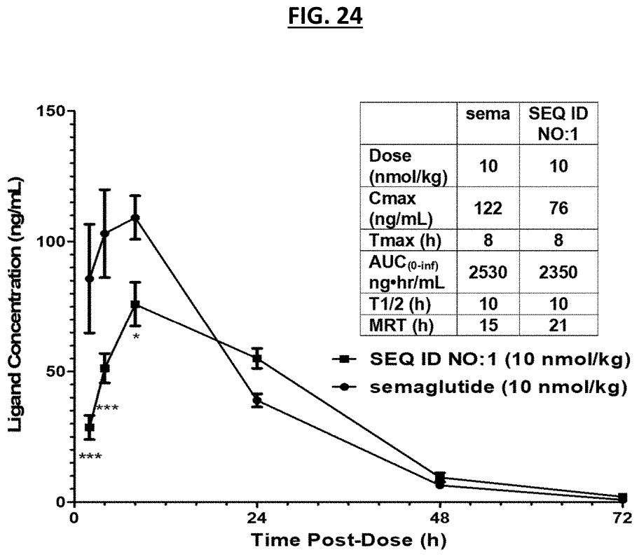

The PK profile for compound 17 was determined initially in comparison to semaglutide in rats following sc administration at 10 nmol/kg. The Tₘₐₓ measured for 17 and semaglutide is 8 h (FIG. 24), although plasma levels of 17 appear to be still rising sharply, indicative of the true Tₘₐₓ of >8 h. The Cₘₐₓ of 17 was 62% of that of semaglutide (76 vs 122 ng/mL) but the AUC was comparable (2,350 vs 2,530 ng·h/mL, respectively). Overall, 17 had a somewhat longer MRT than semaglutide, 21 h and 15 h, respectively. Following sc dosing, the plasma conc of 17 increased dose-proportionally with a 3-fold dose (30 nmol/kg) increase resulting in a 2.8- and 3-fold increase in Cₘₐₓ and AUC, respectively (data not shown). Such a profile, with lower and later Cmax, also was observed in mice (data not shown) and would be expected to provide a lower peak to trough ratio than semaglutide, with the potential for reduced side effects. An iv dose of 10 nmol/kg (data not shown) had a t_{1/2} of 10 h and indicated a bioavailability of 29% for the same dose given subcutaneously, albeit with the limitation of the apparent inaccuracy of the Tₘₐₓ and AUC (see F % for minipigs). FIG. 24 illustrates the in vivo PK behavior of 17 and literature standard semaglutide following subcutaneous administration to CRL: CD (SD) rats at 10 nmol/kg. Analogue 17 shows significantly lower plasma concentrations (*** at t=2 and 4 h; * at t=8 h) and later PK profile in this and other assays, which may translate to a decreased peak/trough ratio. Compared to semaglutide: *, P<0.05; ***, P<0.001.

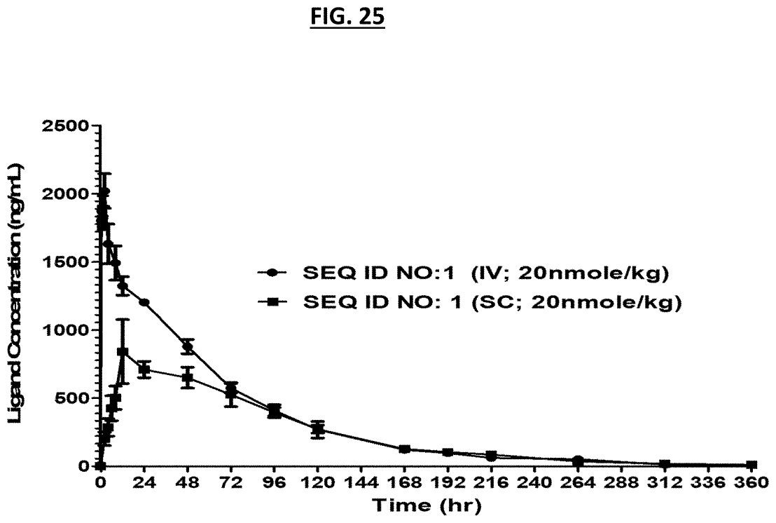

The PK behavior of 17 in a larger animal was examined by iv and sc injection of a single dose of 20 nmol/kg in Yucatan mini-swine (FIG. 25). A very prolonged PK curve was observed (SC, t_{1/2}=52 h; MRT=84 h) with a low Cₘₐₓ (890 ng/mL). The bioavailability of subcutaneous 17 vs intravenous (iv) administration was 73%. The PK behavior of 17 is similar to published reports for semaglutide (sc, MRT=64 h) in Göttingen mini-pigs and 17 is anticipated, similarly, to be suitable for QW (once weekly) administration to patients. There were no clinical observations reported (e.g. evidence for nausea, emesis, or decreased feeding) during this study in adult mini-swine. FIG. 25 illustrates the in vivo pharmacokinetic behavior of 17 following single subcutaneous and intravenous (iv) administration to male mini-swine (n=4; wt circa 75 kg) at 20 nmol/kg. Analogue 17 shows a very prolonged pk profile, somewhat longer than that reported for semaglutide (MRT 86 h vs 64 h, respectively), indicating that 17 is suitable for QW administration in patients.

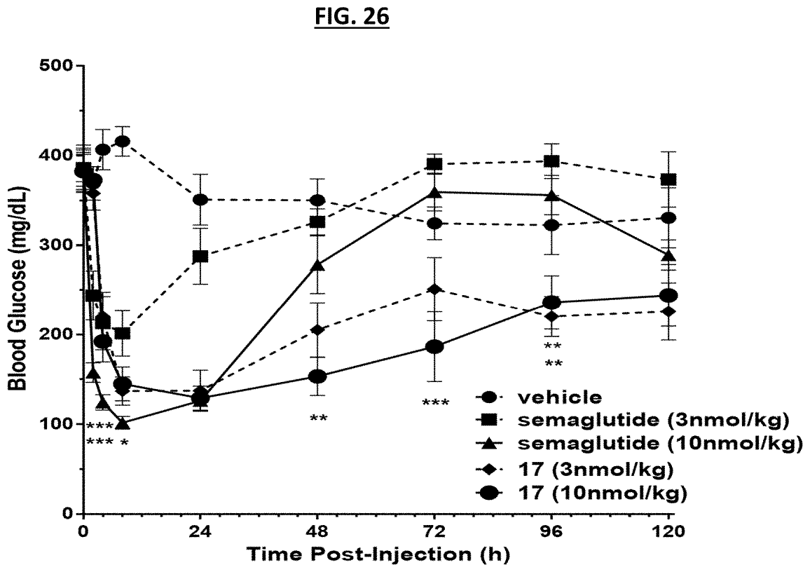

The glucose lowering potency of 17 was initially examined in a dose-finding study in db/db mice vs the literature standard semaglutide (FIG. 26). Semaglutide was not fully effective at 3 nmol/kg while at 10 nmol/kg it caused a precipitous drop in blood glucose, to somewhat below (105 mg/dL) the reference level for a normal C57BL/6J mouse (126 mg/dL) at the 8 h timepoint. Blood glucose was maintained in a near normalized range for high dose semaglutide at 24 h and returned to an elevated level (280 mg/dL) by 48 h. Thus 10 nmol/kg appears to be a fully effective dose for QD semaglutide in this mouse model. The effects of 3 and 10 nmol/kg of 17 were similar to each other acutely, reducing the blood glucose to 129 mg/dL, close to the normal mouse range, with the maximal effect seen at 24 h. The higher dose of 17 (10 nmol/kg) maintains blood glucose in a reduced range (153 and 187 mg/dL) at 48 and 72 h post dose. Blood glucose levels were significantly above those of semaglutide at 2 and 4 h (p<0.0001 and <0.02, respectively) while below those of semaglutide at 48, 72, and 96 h (p<0.01 at each). Thus, in this dose-finding assay in db/db mice, 17 appears to be more potent and longer-acting than semaglutide for glucoregulatory effects, while approaching maximum glucose-lowering in a more gradual way. FIG. 25 illustrates the in vivo dose response behavior of 17 and literature standard semaglutide following subcutaneous administration of single dose, in male db/db mice (n=9). Analogue 17 appears to be more potent, more measured and more prolonged in its PD effect compared to semaglutide, which causes an acute blood glucose decrease to a level below that seen in normal C57BL/6J mice. For equimolar doses of 17 (10 nmol/kg) vs semaglutide (10 nmol/kg), blood glucose levels are significantly different at t=2, 48, 72, and 96 h; *=p<0.05, **=p<0.01, ***=p<0.001.

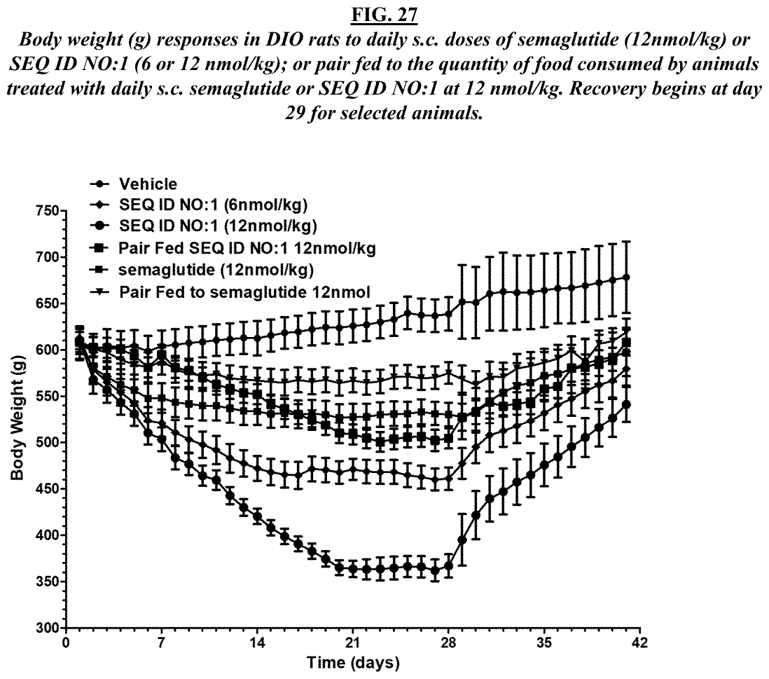

The pharmacodynamic profile of 17 was examined in a 28-day, diet-induced obese (DIO) rat model vs semaglutide as literature standard (FIG. 27). Groups of DIO CD: SD (Sprague-Dawley) rats (n=9) were treated QD subcutaneous with vehicle, 12 nmol/kg semaglutide, 6 or 12 nmol/kg 17, and groups also were pair-fed to the amount of food consumed by the 12 nmol/kg semaglutide or 17 groups. Groups treated with either compound rapidly reached a reduced weight that was stable throughout the assay. Importantly, analogue 17 treatment dose-dependently returned animals to the lean weight range typically observed with moderate to marked dietary restriction (circa 350 to 500 g, indicative of longer survivability) and then maintained that weight. SD rats fed ad libitum are known to develop diabesity, with a shortened life span not suitable for prolonged studies (spontaneous tumors, degenerative diseases), while restricted diets lead to decreased total weight with consistently longer survival. No hyperglycemia was noted and all animals survived to termination. During a 2-week recovery phase, animals (4 per group) in all treated groups rapidly regained weight lost during treatment. FIG. 27 illustrates the body weight of male DIO rats (n=9) during 28 day treatment (followed by recovery) with vehicle, literature standard semaglutide (12 nmol/kg), analogue 17 (6 and 12 nmol/kg), and groups pair-fed to the amount of food consumed by the animals in the 12 nmol/kg semaglutide and 17 groups. Treated groups rapidly reach and maintain stable body weights, then rapidly regain weight during recovery (n=4). Analogue 17 treatment achieved greater body weight loss (−24% and −40%; 6 and 12 nmol/kg, respectively) than semaglutide treatment (−13%). Low dose (6 nmol/kg) 17-treated animals showed significantly lower body weight compared to semaglutide (12 nmol/kg) on days 14-17 (*); days 23-25 (*), days 26-28 (**). Equimolar (12 nmol/kg) 17-treated animals showed significantly decreased weight compared to semaglutide on days 9 (*), 10 (**) and 11-28 (***); *=p<0.05, **=p<0.01, ***=p<0.001.

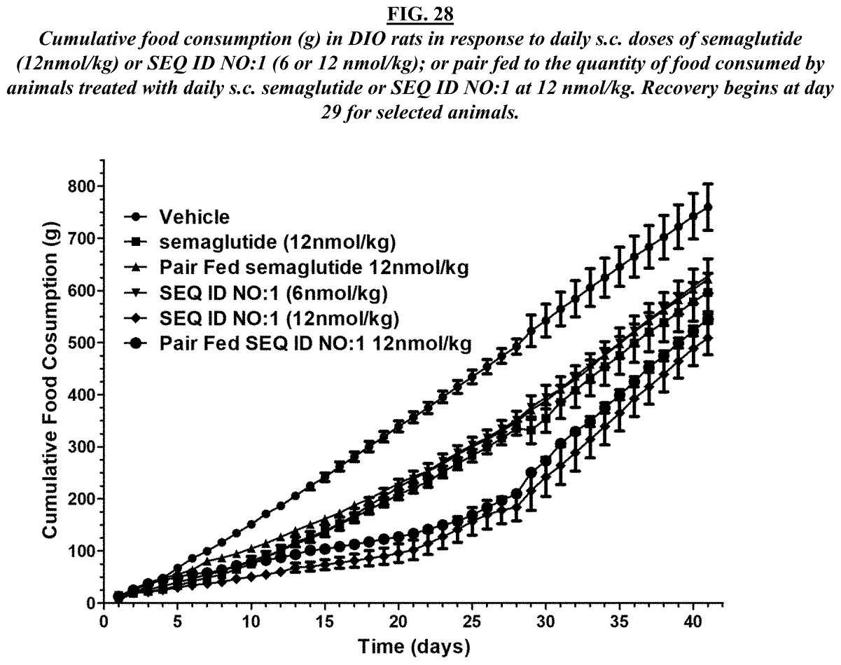

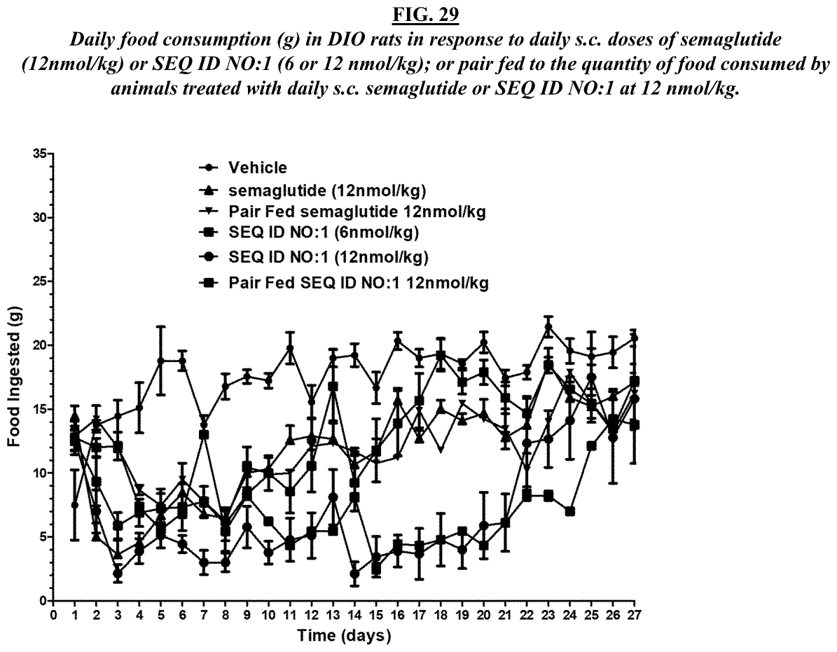

As can be seen in FIG. 28, animals treated with low dose 17 (6 nmol/kg) showed very similar feeding suppression as those treated with semaglutide at twice the molar equivalent dose, but showed circa double the weight loss (−24% vs −13%, 17 vs semaglutide, respectively). This difference is indicative of the second mechanism of action to promote weight loss, GCGR activation. While animals treated with semaglutide showed significant, but transient feeding suppression, equimolar 17-treated animals showed more sustained feeding suppression throughout most of the assay (FIG. 29) and significantly greater body weight loss (−40% vs −13%, 17 vs semaglutide, respectively). FIG. 28 illustrates cumulative food consumption by DIO rats during 27 day treatment (followed by recovery) with vehicle, literature standard semaglutide (12 nmol/kg), analogue 17 (6 and 12 nmol/kg), and groups pair-fed to the amount of food consumed by the animals in the 12 nmol/kg semaglutide or 17 groups. Note low dose 17 and semaglutide achieve a similar degree of feeding suppression early, while 17 at equimolar dose to semaglutide (12 nmol/kg) shows feeding suppression throughout most of the assay. Both 17-treated groups achieve substantially greater body weight loss compared to semaglutide (FIG. 27). Compared to Vehicle, all treated groups showed significantly reduced food consumption beyond day 8, with significance for semaglutide (12 nmol/kg) beginning day 7 and for 17 (12 nmol/kg) on day 6. Treatment with equimolar 17 and semaglutide (12 nmol/kg) showed that 17 caused decreased food consumption vs semaglutide at day 14 (p<0.05), 15 (p<0.01) and 16-28 (p<0.001). The groups pair-fed to semaglutide and to 17 closely matched their intended diminished food consumption but the corresponding treated groups showed greater weight loss (−6% vs −13% and −18% vs −40%; pair-fed vs treated, semaglutide vs 17, respectively), again confirming additional mechanisms of action for both semaglutide and analogue 17. The additional effect on weight loss was modest for the GLP-1 analog semaglutide and very substantial for the GLP-1R/GCGR dual agonist, analogue 17. Studies with other GLP-1/GCGR analogs have implicated increased metabolic rate, white adipose tissue browning and thermogenesis in the increased weight loss seen with such analogs but such studies have had variable results and have not been carried out for 17.

An important aspect in evaluating DIO rat models is the effect on liver weight, since obesity is thought to drive the liver enlargement, steatosis and inflammation of the NAFLD/NASH disease spectrum. In this study the liver weights (and as % of body weight) at 28 days were vehicle (18.6 g, 2.9%), semaglutide (14.9 g; 2.8%), pair-fed to semaglutide (16.5 g, 2.9%), low dose 17 (11.5 g, 2.5%), high dose 17 (8.9 g, 2.4%), pair-fed to high dose 17 (14.3 g, 2.8%). The decreased liver weight in the 12 nmol/kg 17 group was statistically different (p<0.01) from both the vehicle and the equimolar semaglutide groups. In view of the significantly greater decrease in liver weight with 17 it is interesting that while studies with carefully validated antibodies demonstrate the presence of GCGR in the liver, GLP-1R are not seen. While beneficial effects of GCGR agonists in the liver are likely direct, those of GLP-1R agonists on liver weight and histology presumably are due to indirect effects on body weight and lipid levels.

L. Conclusions from Example 6

The rapidly increasing worldwide obesity epidemic is driving a spectrum of metabolic syndrome-associated diseases exemplified by type 2 diabetes and NASH. Existing drugs, including GLP-1 analogs and previously studied GLP-1/GCGR dual agonists, do not adequately address the need for very substantial weight loss (>10%) at approved doses and we sought a substantially more effective and well-tolerated agent with the potential for QW delivery in humans. Based on earlier studies showing substantial prolongation of duration of action for peptides through transient binding to human serum albumin (HSA), we studied the modification of a relatively evenly-balanced GLP-1R/GCGR dual agonist peptide framework with a novel approach, conjugation with functionalized non-ionic glycolipid surfactants, termed EuPort reagents. It is interesting to compare the potency and selectivity of 17 vs that of the peptide framework chosen for investigating the structure activity behavior of this new class of peptide modifier. That peptide sequence (compound 32 in Day, et al. A new glucagon and GLP-1 co-agonist eliminates obesity in rodents. Nat Chem Biol 2009, 5, 749-757) was modified by the widely used polyethylene glycol (40 kDa; PEGylation) approach to yield the long-acting PEGylated molecule focused on therein (compound 33). However, PEGylation typically causes very substantial losses in potency (for 33, a 12 fold loss in GCGR potency and 5 fold loss in GLP-1 potency relative to 32) resulting in that case in loss of selectivity balance (ratio of potencies therein decreased from 0.45 to 0.17, thus favoring GLP-1R and no longer balanced). The studies presented herein also identified a sensitivity of GCGR activation to steric bulk (analogs 9-11). PEGylation also brings issues with respect to characterization (an envelope of molecules with varied molecular weight) as well as concerns with respect to PEG immunogenicity and slowed clearance. In contrast, conjugation with glycolipid surfactants, here, and in a PTH series, yielded prolonged, tunable duration of action with high potency and selectivity, without needing additional linkers. Thus, the relatively rigid presentation to solvent of the lipid tail on a carbohydrate ring system appears to be a favorable new approach in at least two hormone analog series. Detailed evaluation of the physical properties of similarly glycolipid-surfactant modified peptides is of substantial interest.

Seeking a duration of action suitable for QW delivery to patients, we are developing analogue 17, which has demonstrated the desired very high and evenly-balanced potency for activation of cloned human GLP-1R and GCGRs in vitro, return of DIO rodent models to diet-restricted, chow-fed, lean body weight and very high SA binding. The latter aspect results in very prolonged duration of action in rodents and in mini-swine (t_{1/2}=52 h; MRT=84 h), a profile suggesting suitability for QW administration in humans. Benchmarking against the literature standard, QW GLP-1R agonist semaglutide, indicates that dual agonist 17 is more potent, longer-acting and more effective in causing body weight loss in DIO rodent models, returning them to a lean body phenotype. Accordingly, 17 (formulated as ALT-801, formerly known as SP-1373) is currently completing studies to assess its therapeutic potential in treating metabolic diseases such as obesity and NASH.

### Example 7. Clinical Trial to Determine the Safety and Tolerability of Single and Repeated SC Doses of ALT-801 in Healthy Overweight and Obese Subjects, and to Characterize the Effective Dose Range Based on PK-PD Relationships

This study is designed to assess the safety and tolerability of single and repeated SC doses of ALT-801 in healthy overweight and obese subjects (BMI 25.0-40.0 kg/m²) and to characterize the effective dose range based on pharmacokinetic-pharmacodynamic (PK-PD) relationships. Overweight and obese healthy volunteers are studied as the PK in such subjects may be different from that in normal weight individuals. In addition, these subjects are able to better tolerate the predicted PD effect of weight loss and could even benefit from treatment. Appropriate contraceptive measures have been put in place to minimize the chances of pregnancy, and precautions have been taken to exclude pre-existing conditions that would make subjects at risk for treatment with GLP-1 or glucagon analogues. Diabetic subjects have been excluded until the effects of ALT-801 on glucose homeostasis are better characterized in a non-diabetic population. As overweight and obese subjects are expected to have varying levels of insulin resistance, the observations made in these studies, taken together with data from other compounds in this class, should be predictive of the effects observed when diabetic subjects are studied. Exclusions have been instituted that might otherwise affect an accurate assessment of the effects of ALT-801 on safety, PK, or PD. Analyses is conducted to evaluate the effects of the range of BMIs employed in this study on PK and PD parameters. The study will show the effects of ALT-801 on body weight, providing support for its use as a primary treatment for obesity.

The primary objective of the study is to assess the safety and tolerability of ALT-801 in healthy overweight and obese subjects after single and multiple ascending subcutaneous (SC) dose administration, by assessing adverse events (AEs), vital signs, clinical safety labs, urinalysis, physical examination, and injection site reactions; glucose homeostasis; blood pressure; electrocardiogram (ECK), Holter monitoring; and the like. The secondary objectives of the study are to evaluate: 1) the PK of ALT-801 after single and multiple ascending SC dose administration; and, 2) the PD effects of ALT-801 after single and multiple dose administration. Exploratory objectives of the study include evaluation of: 1) the expanded PD effects of ALT-801 after multiple dose administration; and, 2) the effects of ALT-801 on heart rate-corrected QT interval (QTc) prolongation. The study assessments, including liver fat content by MRI-PDFF, body weight, body composition by whole body MM, insulin resistance, systemic inflammation, and GLP-1 and glucagon target engagement are based on the expected PD properties of ALT-801, which include weight loss and change in body composition. Measurements of glucose homeostasis are based on the potential effects of GLP-1 and glucagon analogues on glucose control. Ambulatory blood pressure monitoring (ABPM) and Holter monitoring have been included since GLP-1 and glucagon agonists have been associated with clinically insignificant changes in blood pressure and heart rate. Holter monitoring has also been included to provide information on any potential effects of ALT-801 on QT interval prolongation. Based on the pharmacology and safety experience with GLP-1 and GLP-1/glucagon dual agonists, a dose-related incidence of GI AEs, including nausea and vomiting, may occur. Glucose homeostasis will also be evaluated, including the incidence and severity of hyperglycemia and hypoglycemia. As weight loss is a desired property of this compound, it is monitored for efficacy rather than safety. However, if weight loss is deemed to be excessive, the dose in subsequent cohorts may be adjusted. Study medication may be paused or discontinued in individual subjects if the level of weight loss is considered unsafe or excessive. Subjects will also be monitored for drug-induced liver injury. A blood sample is collected predose and after the final dose of study drug for biobanking in subjects that provide separate consent. These samples are used to discover and/or validate biomarkers in NASH and related diseases, including potential genetic analyses.

This study described herein is a first-in-huma (FIH), Phase 1, randomized, double-blind, placebo-controlled, 2-part study of single ascending doses (SAD) and multiple ascending doses (MAD) of ALT-801 in healthy overweight and obese subjects. Overweight to obese subjects (body mass index [BMI] 25.0-40.0 kg/m2) will be enrolled. In Part 1, the single ascending dose (SAD) Phase, subjects undergo a screening period of up to 28 days. Overweight to obese subjects who meet inclusion and do not meet exclusion criteria will be randomized in a 3:1 ratio in cohorts of 8 subjects, with 6 subjects to receive ALT-801 and 2 subjects to receive placebo. Study medication (SEQ ID NO: 1 formulated as ALT-801 for subcutaneous (SC) administration) is administered subcutaneously (SC) at abdominal sites in all SAD cohorts. Subjects are admitted to the research unit approximately 1 day prior to study medication administration (Day −1) and will be discharged on Day 8. Subjects will receive 1 SC dose of ALT-801 or placebo on Day 1. Six cohorts are planned, with 2 additional optional cohorts, for Part 1. The following dose levels are planned: 0.4, 1.2, 2.4, 4.8, 7.2, and 9.4 mg as a weekly dose administered once a week (QW) based on a 60 kg human. These doses may be modified on the basis of clinical observations, or, when available, PK data. The first 2 subjects (1 ALT-801 and 1 placebo) in each SAD cohort are dosed in sentinel manner at least 48 hours before the remaining subjects. Subjects undergo overnight fasting for at least 10 hours prior to assessments on Days −1 through 5 and prior to assessments on Day 8, and meals will be standardized. Subjects undergo study assessments to evaluate safety, including ECGs, CGM, and ABPM, and will have blood samples collected for PK as described in the schedule of assessments as described below. Following discharge from the research unit, subjects will return for outpatient visits for PK and safety assessments every 3 days through Day 26 and for a follow-up visit on Day 35 or at least 5 half-lives, as determined over the course of dosing. If predicted efficacious doses and exposures based on pharmacometric modeling are not achieved and/or if the maximum tolerated dose (MTD) for a single dose is not identified after completing the 6 planned cohorts, up to 2 additional single-dose cohorts are enrolled in Part 1. Part 2, the multiple ascending dose (MAD) phase commences once Day 8 of SAD Cohort 3 is completed and the safety of that cohort is assessed. The starting dose in Part 2 is one-half the dose for SAD Cohort 3.

After providing informed consent, overweight to obese subjects undergo a screening period of up to 28 days. Subjects are instructed to maintain their normal diet and activities during screening and not to start any new diets, supplements, or exercise programs at any time while participating in the study. Subjects are admitted to the research unit approximately 4 days prior to study medication administration (Day −4) for a diet and exercise run-in acclimation period during which they will receive standardized meals. A standardized diet is provided with daily calories individualized using a predictive BMR×1.5 to account for inter-subject differences based on body weight, height, age, and sex. The activity level of study participants is also standardized. Subjects who meet inclusion and do not meet exclusion criteria are randomized on Day −1 in a 5:1 ratio in cohorts of 12 subjects, with 10 subjects to receive ALT-801 QW and 2 subjects to receive placebo QW for 6 weeks. Study medication is administered subcutaneously (SC) at abdominal sites in all MAD cohorts.

Subjects receive the first dose of study medication on Day 1 and remain in the research unit until after they receive the second dose on Day 8. Subjects then return for 3 outpatient dosing visits at weekly intervals (Days 15, 22, and 29) and are re-admitted from Day 32 to Day 43. Subjects will receive the last dose of study medication on Day 36. Following discharge on Day 43, subjects return for a follow-up visit on Day 70 or 5 half-lives after dosing, whichever is sooner. Subjects undergo several study assessments to evaluate the safety, PD, and PK of ALT-801 as described herein. Safety evaluations will include ECGs, CGM, and ABPM. PD assessments include anthropomorphic measures, dietary assessments, imaging, and blood collection for biomarkers. The Patient Assessment of Gastrointestinal Disorders Symptom Severity Index (PAGI-SYM) is administered to assess the effects of treatment on GI symptoms. Blood samples are collected for PK and immunogenicity. Subjects undergo overnight fasting for at least 10 hours prior to Day −1 through Day 5 and prior to Days 7, 8, 36, 37, 42, and 43. In addition, subjects will receive a standard breakfast meal for the mixed meal tolerance tests on Days −1, 7, and 42.

The doses for the MAD will be selected on the basis of clinical data and, when available, PK data from previously completed SAD and MAD cohorts. Three MAD cohorts are planned with up to 2 optional additional cohorts, if needed, to achieve predicted efficacious doses and exposures based on pharmacometric modeling, to expand a previously studied dose level, to continue dose escalation if an MTD for this phase is not identified, or explore dose titration schemes if GI intolerance is observed before the maximally effective dose based on pharmacometric modeling is reached.

The maximal recommended starting dose (MRSD) in Part 1 is based on one tenth the human equivalent dose (HED) at the NOAEL determined in animals (rats and monkeys) in the pivotal Good Laboratory Practice toxicology study. Both rats and monkeys were deemed to have a similar clinical response to ALT-801 (see Example 4), but the exposures at the NOAEL were slightly lower in rats, resulting in a more conservative human starting dose. The rat NOAEL was the high dose, 0.45 mg/kg/week, which is equivalent to 0.44 mg/wk in a 60 kg human based on body surface area scaling. Notably, the NOAEL in monkeys was also the high dose, 0.25 mg/kg, which is equivalent to 0.49 mg/wk in a 60 kg human based on body surface area scaling. Using a 10-fold scaling factor for safety, the human starting dose of 0.40 mg/wk for a 60 kg human was selected. Furthermore, extrapolated human exposures at the maximum recommended starting dose (MRSD) are well below the exposures at the monkey NOAEL, which notably, are comparable to exposures at the rat NOAEL This is particularly relevant because the monkey, although not the most sensitive species, is biologically the more relevant species for the most clinically relevant toxicities (ie, reduced food intake and vomiting). Clinical observations and PK in Part 1 will ultimately guide dosing considerations in Part 2.

The primary findings of ALT-801 in studies in rats and monkeys was weight loss (see, e.g., Example 4). Modifying the schedule in rats, which were dosed daily, to 3 days a week, improved tolerability by reducing the impact of ALT-801 on food consumption and body weight loss, consistent with the mechanism of action (see Example 4). The toxicity of GLP-1 and glucagon agonists have also been well characterized in human studies. The pre-clinical safety findings support a 3-fold dose escalation increment to SAD Cohort 2. Subsequent escalations will not exceed 2-fold in either part of the study. Dose titration schemes may be explored if needed to improve tolerability. Adding to the confidence around these predictions, the dose-exposure relationship in humans is predicted to be linear based on a population PK model of several preclinical species (mice, rats, mini-pigs, and monkeys), as described in Example 4. The model is updated with human data as the study is ongoing. The predicted t_{1/2} of ALT-801 in humans is in the range of 100 hours, an assumption that will also be confirmed in Part 1. Based on once-weekly (QW) dosing, the estimated accumulation with repeated dosing at steady state is not greater than 2-fold. To ensure that multiple dose exposures will remain within single dose exposures, the starting dose in Part 2 is planned to be one-half the dose for Part 1 Cohort 3. However, subsequent Part 2 cohorts may be adjusted based on safety and PK data. The decision to escalate to each successive dose level is based on assessment of safety and tolerability through Day 8 (7 days following the single dose) in Part 1 and Day 15 (7 days following the second dose) in Part 2. The decision to dose-escalate after the second week is completed is based on the observation from earlier GLP-1 and GLP-1/glucagon dual agonist studies that AEs, which are expected to be predominately nausea or vomiting, will occur in the first 2 weeks of dosing. Further, the expectation is the Cₘₐₓ and AUCₜₐᵤ of the final week of dosing will not exceed the Cₘₐₓ or AUC_{inf} of a dose in a previously completed and safety-assessed SAD cohort. The target dose for maximal efficacy, corresponding to ED80 to ED90, in an adult human is estimated to be between 1 and 5 mg, and the target plasma concentrations between 50 and 100 ng/ml, based on exposures in animals at efficacious doses and pharmacometric modelling of animal PK parameters to predict human PK. Thus, the estimated starting dose is approximately 2.5-fold lower than the lowest predicted efficacious dose and is expected to be inactive.

To maximize safety, single ascending dose (SAD) and multiple ascending dose (MAD) escalation is planned to not exceed exposures at the NOAEL in rats. However, if PD and tolerability suggest that overweight and obese subjects would benefit from doses that are expected to exceed exposures at the rat NOAEL, supportive safety and efficacy data is presented to the IEC and agreement is obtained prior to continuing SAD and MAD escalation.

A minimum of 6 subjects is required to dose escalate in Part 1, and 8 subjects in Part 2, with at least 1 subject in each cohort receiving placebo. The suggested next dose levels may be adjusted downward based on evaluation of safety and tolerability data observed in previous treatment cohorts if observations suggest that dose escalation is exceeding MTD. Dosing may proceed until the MTD is identified, which is determined separately for each part of the study. Available PK data may be used to guide decision making and is explicitly considered if exposures are expected to exceed the NOAEL in rats. To maximize safety, the planned SAD and MAD escalation will not exceed exposures at the NOAEL in rats.

Following completion of the screening activities, subjects who meet the all the inclusion (e.g., and none of the exclusion criteria are randomized by an interactive web response system (IWRS). In Part 1, 2 subjects in each cohort are randomly assigned 1:1 to ALT-801 or placebo treatment groups for sentinel dosing. The remaining 6 subjects in each cohort of 8 subjects are randomly assigned to ALT-801 or placebo treatment groups, with 5 assigned to the ALT-801 group and 1 assigned to the placebo group for an overall 3:1 ratio of ALT-801 and placebo in each cohort. In Part 2, cohorts of 12 subjects are randomly assigned in a 5:1 ratio to ALT-801 or placebo treatment groups, with 10 assigned to the ALT-801 group and 2 assigned to the placebo group.

ALT-801 is formulated in glass vials in a sterile, buffered aqueous solution to a final concentration of 2.5 mg/mL and total fill volume of 1.2 mL, and administered as a subcutaneous (SC) injection. In Part 1, a single dose of study medication is administered on Day 1. The first 2 subjects (1 ALT-801 and 1 placebo) in each SAD cohort is dosed in sentinel manner at least 48 hours before the remaining subjects. In Part 2, study medication is administered QW for 6 weeks. Doses are administered on Days 1, 8, 15, 22, 29, and 36. The starting dose in Part 1 is 0.40 mg, which corresponds to one-tenth the human equivalent dose at the no observed adverse effect level (NOAEL) in rats (rounded down from 0.44 mg/wk for safety), and the dose escalation will follow a modified Fibonacci scheme and is 3-fold or less with planned dose levels of 0.40, 1.2, 2.4, 4.8, 7.2, and 9.4 mg (equivalent to a weekly dose administered once every 7 days). The starting dose in Part 2 is planned to be one-half the dose for Part 1 Cohort 3. However, subsequent Part 2 cohorts may be adjusted based on safety and PK data. The decision to escalate to each successive dose level is based on assessment of safety and tolerability through Day 8 in Part 1 (7 days following the single dose) and Day 15 (7 days following the second dose) in Part 2. Dose escalation may be modified, and dose titration schemes as appropriate, or as described herein. Each dose of ALT-801 or placebo is administered as a SC injection in the abdominal region by appropriately trained clinical staff members. The volume of administration is based on the selected dose and a concentration of 2.5 mg/mL for the final drug product. The saline placebo is matched for volume based on the dose and volume of ALT-801 administered in that cohort. As weight loss is a desired property of this compound, it is monitored for efficacy rather than safety. However, if weight loss is deemed to be excessive, the dose in subsequent cohorts may be adjusted. Study medication may be paused or discontinued in individual subjects if the level of weight loss is considered excessive. Study medication may be paused or discontinued in individual subjects if the level of GI adverse events is considered excessive and intolerable despite antiemetic treatment (eg, severe GI AEs continue >24 hours). If there is persistent vomiting a subject may be given an antiemetic. A 5HT3 receptor antagonist (eg, ondansetron) is preferable in this situation. The suggested dose levels may be adjusted downward based on evaluation of safety and tolerability data observed in previous treatment cohorts if observations suggest that dose escalation is exceeding the MTD. Dosing may proceed until the MTD is identified, which is determined separately for each part of the study. Available PK data may be used to guide decision making.

Blood samples are collected for PK assessment at hour zero, 1, 4, 6, 8, 12, and 16 on days −1, 1, 2, 3, 4, 5, 8, 11, 14, 17, 20, 23 and 26 for Part 1 and hour zero, 1, 4, 6, 8, 12, and 16 on days −1, 1, 2, 3, 4, 5, 8, 15, 22, 29 and 36-38 for Part 2. Remaining plasma from PK samples may be stored frozen with no time limitation and may be used for ALT-801 bioanalytical method development or to explore ALT-801 metabolites. ECG readings are time-matched to the PK sample times. When multiple activities occur at the same timepoint, ECGs should be collected first, and PK blood draws should occur at the nominal time. PD assessments are done in Part 2 only.

Height is measured in centimeters using a wall-mounted stadiometer or one mounted on a balance beam scale, whichever is available. Subjects should be wearing socks or be barefoot. With the exception of Screening visits, weight is measured in kilograms using a calibrated scale at approximately the same time of day at each nominal timepoint. Measurements should be taken with subjects wearing a gown (or other standard clothing provided by the clinical research unit), undergarments, and socks (no shoes), while fasting and after the subject has been asked to void (ie, empty bladder). Waist circumference should be taken with the subject wearing a gown. The measurement is performed at a level midway between the superior aspect of the iliac crests and the lower lateral margin of the ribs. The measurement need not be at the level of the umbilicus. The measuring tape is kept horizontal. Height, weight, and waist circumference is measured and BMI calculated and recorded according to the schedules in Part 1 and Part 2. Measurement of height is required at screening only. Waist circumference is measured for subjects in Part 2 only.

FibroScan® is an ultrasound-like instrument able to simultaneously measure liver stiffness and steatosis through Vibration-Controlled Transient Elastography (VCTE) and CAP, respectively. For subjects in Part 2, FibroScan® CAP is measured during screening following an overnight fast of at least 10 hours. FibroScan® CAP is measured before MRI-PDFF. MRI-PDFF is a quantitative imaging biomarker that enables accurate, repeatable and reproducible quantitative assessment of liver fat over the entire liver. For subjects in Part 2, MRI-PDFF is measured during screening (only occurs if CAP is ≥300 dB/m) and at the EOS visit following a minimum 10 hour fast. The percent liver fat is corrected for total liver volume, which is measured simultaneously with liver fat content. Whole body MRI is an established imaging technique that is used to measure body composition, including lean body mass. For subjects in Part 2, whole body MM is performed during screening and the EOS visit in conjunction with MRI-PDFF.

In Parts 1 and 2, subjects are provided a standardized diet during the inpatient periods at the research unit. Daily calories are individualized using a predictive BMR equation multiplied by an activity factor of 1.5 and macronutrient composition is standardized at 40-50% carbohydrate, 15-25% protein, and 30-40% fat. In Part 2, the same standardized meals are provided on Day −4 to Day −2 and Day 39 to Day 41, prior to PD assessments on Day −1 and Day 42. The timing and type of meals will also be specific for ECG, MRI-PDFF, and MMTT assessments, as described in each of the corresponding manuals.

Food intake and appetite are assessed using an ad libitum meal test and the VAS questionnaire. VAS questionnaires are standard techniques in appetite research that record feelings of hunger, satiety, fullness, and desire to eat specific tastes, such as sweet, salty, savory, and fatty [Flint 2000]. Subjects will complete a VAS questionnaire before and after an ad libitum meal on days specified in the schedule of assessments. The size of the ad libitum meal will exceed expected intake of healthy overweight and obese volunteers. During the test meal, subjects are isolated and environmental cues minimized (ie, no TV, cell phones, computers, etc.). Subjects are instructed that they have 30 minutes to consume as much or as little as they want, and they should eat until comfortably full. Pre and post meal weights are recorded to capture food intake, and caloric consumption is determined.

The basal metabolic rate (BMR) and resting energy exposure (REE) are assessed in the morning under fasting conditions and following a fasting period of at least 10 hours. Resting energy expenditure to be conducted on Days −1 and 42. BMR and REE are determined using the ventilated hood method (indirect calorimetry). Because BMR usually is the main component of daily energy expenditure, changes to BMR might be of clinical relevance within the context of a metabolic drug development program that targets energy expenditure.

Following a minimum 10 hour fast the subject will undergo a mixed meal tolerance test (MMTT) which will involve the consumption of a standardized liquid meal (6 fluid ounces of Ensure Plus [700 kcal], a nutritional supplement containing the components of fat, carbohydrate, and protein, which make up a standard MMTT) within 5 minutes. The t=0 minute sample (i.e. prior to the standardized liquid meal) are the last HOMA IR 2 blood sample (see above). Hormone markers will include glucose, insulin and C-peptide. Samples are collected at intervals of 5 minutes for the first 15 minutes and 30 minutes thereafter through 240 minutes after consumption of the standardized liquid meal (with no additional food intake during this time). The MMTT procedures are performed on days specified in the schedule of assessments. In order to standardize the test and reduce variability, each test is preceded by a 3-day standardized diet and standardized physical activity run-in period after admission to the clinical research unit.

Blood samples are collected for evaluation of ketone bodies after the subject has fasted overnight for at least 10 hours, 1 day prior to the first and second doses, and 6 days after the last dose. Blood samples for evaluation of FGF-21 and adiponectin are collected after the subject has fasted overnight for at least 10. Following a minimum 10 hour fast, blood is collected for assessment of lipids, including cholesterol (total, HDL, LDL), Apo A and B, lipoprotein (a), TG, and tripalmitin, prior to the first dose and 6 days after the last dose of study medication, as indicated in Table 4. Blood is collected for the assessment of inflammatory markers, including TNF-α, hs-CRP, leptin, MCP-1, and IL-6 prior to the first dose and 6 days after the last dose of study medication, as indicated in Table 4. Glucose homeostasis is assessed by 24-hour CGM using a Dexcom G6 CGM during the periods indicated in Part 1 and Part 2.

The Safety Population includes all randomized subjects who receive at least 1 dose of study medication. Subjects is analyzed according to the treatment that they receive. The PK Population includes all randomized subjects who receive at least 1 dose of ALT-801 and who have sufficient PK data for analysis. The QT Population includes all subjects in the PK Population who have at least 1 time-matched ECG at baseline and corresponding time-matched PK-ECG postdose. The PD Population includes all randomized subjects who receive at least 1 dose of study medication and who have results from baseline and at least 1 post-baseline PD assessment.

In the statistical methods used, descriptive statistics are used to evaluate differences in demographic and baseline characteristics. Medical history is coded using the most current Medical Dictionary for Regulatory Activities (MedDRA) version and is listed by subject. Continuous safety data is summarized with descriptive statistics (arithmetic mean, standard deviation [SD], median, minimum, and maximum) by dose level and treatment (active or placebo). Categorical safety data is summarized with frequency counts and percentages by study part, dose level, treatment, and day where applicable.

AEs are coded using the most current MedDRA version. A by-subject AE data listing, including verbatim term, preferred term, SOC, treatment, severity, and relationship to study medication, are provided. The number of subjects experiencing treatment-emergent AEs (TEAEs) and number of individual TEAEs and injection site reactions are summarized by treatment group, SOC, and preferred term. TEAEs will also be summarized by severity (Grade 1 through 4) and by relationship to study medication (unlikely, possibly, probably). Relatedness for Stopping Rules are defined as possibly or probably related. Laboratory evaluations, vital signs assessments, continuous cardiac monitoring, ECG parameters (excluding Holter monitoring), CGM measurements, ABPM measurements, and meal tolerance test parameters are summarized by study part, treatment group, dose level, and protocol specified collection time point. A summary of change from baseline at each protocol specified time point by treatment group will also be presented. Changes in physical examinations are listed for each subject. The analysis of the PAGI-SYM is detailed in the statistical analysis plan (SAP). Concomitant medications are listed by subject and coded using the most current WHO drug dictionary.

Pharmacokinetics includes individual ALT-801 concentration data listed and summarized by cohort with descriptive statistics (sample size [N], arithmetic mean, SD, coefficient of variation [CV %], median, minimum, and maximum). Individual and mean±SD ALT-801 concentration-time profiles for each cohort will also be presented graphically. Plasma ALT-801 noncompartmental (NCA) PK parameters Cmax, time to maximum plasma concentration (Tmax), AUC0-t, AUC0-inf, elimination rate constant (Kel), t1/2, apparent total body clearance (CL/F), and apparent volume of distribution during terminal phase (Vz/F) (where data are sufficient for parameter determination) is estimated for the SAD part. For the MAD part, Tmax, Cmax, and AUCtau PK parameters are estimated following the first and the last dose (Week 1 and Week 6). If data permit, Kel, t1/2, apparent total body clearance at steady state (CLSS/F) and apparent volume of distribution at steady state (VSS/F) are estimated following Week 6 dosing. Pharmacokinetic parameters are listed for each individual and summarized by cohort using descriptive statistics (N, arithmetic mean, SD, CV %, median, minimum, maximum, geometric mean, and geometric CV %). The effects of baseline BMI on PK parameters are evaluated by correlation analyses. Dose proportionality is assessed using the power model approach, as appropriate. Accumulation is assessed as the ratio of Cmax and AUC0-tau at Week 6 to Week 1. Steady state is assessed by comparison of trough concentrations from the first to the last dose.

ECGs extracted from Holter monitors are analyzed by a central ECG laboratory with a selected group of skilled readers blinded to subject, visit, treatment, and nominal timepoint. A single reader will review an individual subject's ECGs, unless a second review based on quality control or availability is needed. All ECGs are analyzed using the same lead for an individual subject. The primary analysis lead is Lead II, unless not analyzable, then V2 or V5 is used for an individual subject's entire data set.

The primary analysis is the mean change and one-sided upper 95% confidence limit for the placebo-corrected, change from baseline postdose timepoint using the Fridericia corrected QT interval (AAQTcF). Other correction methods such as Bazett's (QTcB), individual corrected (QTcI), or population corrected (QTcP) may be explored and compared. At minimum, Fridericia's and Bazett's corrections are analyzed and presented. Secondary analyses will include the relationship between time-matched plasma concentrations and AAQTcF using linear mixed effects modelling. The immunogenicity of repeated dose administration of ALT-801 is assessed by evaluation of serum samples using an ELISA based assay collected at the final visit of the MAD phase. If end of study samples are positive, mid-study samples will also be analyzed. Immunogenicity may be correlated to safety and PK, if applicable.

Pharmacodynamics studies include changes in liver fat content, anthropomorphic parameters, GLP-1 engagement and insulin resistance, glucagon engagement, and lipid and inflammation markers are listed and summarized by treatment group with descriptive statistics (sample size [N], arithmetic mean, SD, median, minimum, maximum, geometric mean, and geometric CV %). Inferential statistics are applied, as applicable. The effects of baseline BMI on PD parameters are evaluated by co-variate analyses.

An interim analysis may be conducted following the completion of 2 or more doses in MAD Cohort 3. The objective of this analysis is to permit dose-selection for follow-on trials. For this analysis, the study will remain blinded and subject level safety, PD, and available PK data is de-identified for analysis. Summary data by study part, dose level, treatment group (active or placebo), and by day where applicable is reported. The conduct of the interim analysis is detailed in the SAP.

### Example 8. Formulation Studies

To support clinical development and future commercialization, the formulation of ALT-801 need to be developed to achieve long term stability ideally at +2-8° C. or above. Moreover, formulation of ALT-801 may be optimized to improve pharmacokinetics parameters.

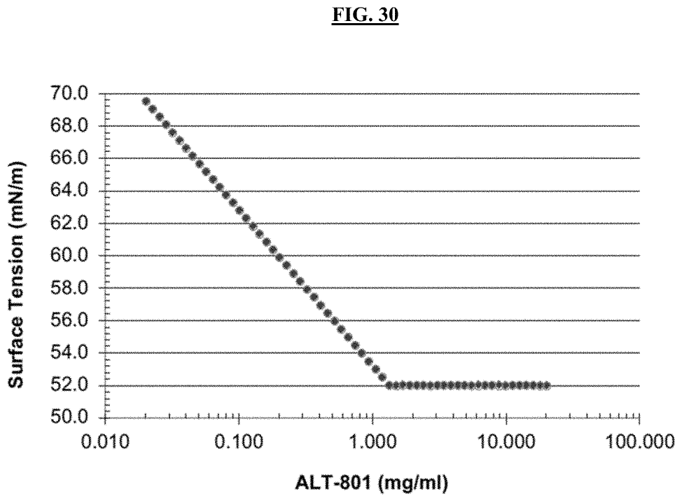

An initial formulation of ALT-801 (F58) disclosed above and containing 2.5 mg/mL of ALT-801 as the API, 3.48 mg/mL Arginine, 0.5 mg/mL Polysorbate 20 (PS-20), and 42.6 mg/mL Mannitol, pH adjusted to ˜7.75 with Hydrochloric acid was developed to support the early clinical development. F58 is stored at −20° C. The F58 formulation was found to become hazy at +2-8° C., indicating larger aggregates were precipitating from solution. When analyzed by RP-HPLC, it was found that purity and content of ALT-801 were unchanged, supporting the hypothesis that this hazy appearance related to physical instability of supramolecular structures formed by ALT-801 in solution. ALT-801 is a peptide amphiphile formed by the covalent attachment of the hydrophobic alkyl chain of EuPort (e.g., functionalized non-ionic glycolipid surfactant) to the hydrophilic peptide portion. As such, ALT-801 is intended to self-assemble into supramolecular structures such as micelles. ALT-801 in water was demonstrated to form micelles at concentrations above the Critical Micelle Concentration (CMC) of 1.33 mg/ml as measured by surface tensiometer (see FIG. 30). The CMC of ALT-801 is expected to be the same in the F58 buffer (without PS-20).

To improve the formulation of ALT-801 for subcutaneous administration, Critical Micelle Concentration (CMC) experiments were assessed by surface tensiometry for both Polysorbate 20 and Polysorbate 80, each in prepared F58 buffer at pH=7.7, under four conditions: alone, with 2.5 mg/ml added ALT-801, with 5.0 mg/ml added ALT-801, and with 10.0 mg/ml added ALT-801. CMC values, and thereby shifts due to ALT-801, and the extent of interaction between Polysorbate 20 or Polysorbate 80 and ALT-801 were determined as presented in Tables 19 and 20, respectively. CMC shifts are simply calculated as CMC in the presence of the ALT-801 less the CMC for the surfactant alone in solution. Extent of interaction on a mass or mole basis are calculated as CMC shift divided by the concentration of the ALT-801 causing the shift.

TABLE 19
CMC, and CMC Shift and extent of interaction between PS-20 and ALT-801

| | Polysorbate 20 | Polysorbate 20 | | Polysorbate 20 | | Polysorbate 20 | |
|---|---|---|---|---|---|---|---|
| | CMC Value | CMC Value | | CMC Value | | CMC Value | |
| | No | 2.5 mg/ml | | 5.0 mg/ml | | 10.0 mg/ml | |
| | ALT-801 | ALT-801 | | ALT-801 | | ALT-801 | |
| Test # | (mg/ml) | (mg/ml) | | (mg/ml) | | (mg/ml) | |
| 1 | 0.722 | 2.37 | | 4.02 | | 7.29 | |
| 2 | 0.721 | 2.36 | | 4.00 | | 7.30 | |
| Average | 0.722 | 2.37 | | 4.01 | | 7.30 | |
| CMC Shift | NA | 1.65 | mg/ml | 3.29 | mg/ml | 6.58 | mg/ml |
| Due to ALT-801 | | | | | | | |
| Extent of Interaction | NA | 0.660 | mg/mg | 0.658 | mg/mg | 0.658 | mg/mg |
| mg PS20/mg ALT-801 | | | | | | | |
| Extent of Interaction | NA | 2.08 | mole/mole | 2.08 | mole/mole | 2.08 | mole/mole |
| moles PS20/molesALT-801 | | | | | | | |

TABLE 20
CMC, and CMC Shift and extent of interaction between PS-80 and ALT-801.

| | Polysorbate 80 | Polysorbate 80 | | Polysorbate 80 | | Polysorbate 80 | |
|---|---|---|---|---|---|---|---|
| | CMC Valve | CMC Value | | CMC Value | | CMC Value | |
| | No | 2.5 mg/ml | | 5.0 mg/ml | | 10.0 mg/ml | |
| | ALT-801 | ALT-801 | | ALT-801 | | ALT-801 | |
| Test # | (mg/ml) | (mg/ml) | | (mg/ml) | | (mg/ml) | |
| 1 | 0.183 | 2.76 | | 5.33 | | 10.50 | |
| 2 | 0.182 | 2.75 | | 5.35 | | 10.49 | |
| Average | 0.183 | 2.76 | | 5.34 | | 10.50 | |
| CMC Shift | NA | 2.58 | mg/ml | 5.16 | mg/ml | 10.32 | mg/ml |
| Due to ALT-801 | | | | | | | |
| Extent of Interaction | NA | 1.03 | mg/mg | 1.03 | mg/mg | 1.03 | mg/mg |
| mg PS80/mg ALT-801 | | | | | | | |
| Extent of Interaction | NA | 3.05 | mole/mole | 3.05 | mole/mole | 3.05 | mole/mole |
| moles PS80/molesALT-801 | | | | | | | |

These results identify the minimum concentration of PS-20 or PS-80 to be used across a range of ALT-801 concentration to achieve its CMC. It also established that the concentration of PS-20 (0.5 mg/ml) in the F58 formulation is too low to achieve the CMC and may explain the hazy appearance of the solution when stored at +2-8° C. The results indicate that at least 0.66 mg of PS-20 is required per mg of ALT-801 to achieve the CMC. Similarly, at least 1.03 mg of PS-80 is required per mg of ALT-801 to achieve the CMC.

Other advantages of the reagents and methods of using the same are also provided herein, as would be understood by those of ordinary skill in the art. While certain embodiments have been described in terms of the preferred embodiments, it is understood that variations and modifications will occur to those skilled in the art. Therefore, it is intended that the appended claims cover all such equivalent variations that come within the scope of the following claims.

1. (canceled)

2. A pharmaceutical dosage formulation comprising an agonist peptide with affinity for glucagon-like peptide 1 receptor (GLP-1R) and glucagon receptor (GCGR) wherein: the peptide is modified with a non-ionic glycolipid surfactant; the dosage is configured to induce weight loss with reduction of one or more adverse events as compared to an agonist with unbalanced affinity for GLP-1R and GCGR, the adverse events being selected from nausea, vomiting, diarrhea, abdominal pain and constipation, upon administration to a mammal.

3-7. (canceled)

8. The pharmaceutical dosage formulation of claim 2 wherein the dual agonist peptide is any one of SEQ ID NOS: 1-10 or 12-27.

9. (canceled)

10. The pharmaceutical dosage formulation of claim 2, wherein the surfactant is a 1-alkyl glycoside class surfactant.

11-17. (canceled)

18. The pharmaceutical dosage formulation of claim 2, wherein the concentration of the dual peptide agonist is 0.05 to 20 mg/ml.

19-27. (canceled)

28. The pharmaceutical dosage formulation of claim 2 configured such that the time to reach a therapeutic dose is about four weeks or less.

29. The pharmaceutical dosage formulation of claim 28 wherein the therapeutic dose exhibits a Cₘₐₓ of from about 10 to about 300 ng/ml; a Tₘₐₓ of from about 10 to about 36 hours; and/or, an AUC₀₋₁₆₈ of from about 1,000 to 100,000 h*ng/mL.

30. (canceled)

31. A method for inducing weight loss in a mammal, the method comprising administering pharmaceutical dosage formulation of claim 2 to a mammal, wherein the method reduces the incidence of one of more adverse events as compared to an agonist with unbalanced affinity for GLP-1R and GCGR, the adverse events being selected from nausea, vomiting, diarrhea, abdominal pain and constipation, upon administration to a mammal.

32-34. (canceled)

35. The method of claim 31, wherein the pharmaceutical dosage is administered is administered subcutaneously.

36-48. (canceled)

49. A pharmaceutical dosage formulation configured for subcutaneous administration comprising an agonist peptide with affinity for glucagon-like peptide 1 receptor (GLP-1R) and glucagon receptor (GCGR) wherein the peptide product is represented as SEQ ID NO: 1; the dosage is configured to induce weight loss with reduction of one or more adverse events as compared to an agonist with unbalanced affinity for GLP-1R and GCGR, the adverse events being selected from nausea, vomiting, diarrhea, abdominal pain and constipation, upon administration to a mammal.

50. The pharmaceutical dosage formulation of claim 49, wherein weight loss is at least 5%, at least 10%; or from about 1% to about 20%; or from about 5% to about 10% (w/w).

51. The pharmaceutical dosage formulation of claim 49, wherein the dosage is configured as a weekly dosage form, optionally configured for administration from about 2 weeks to about 8 weeks.

52. (canceled)

53. The pharmaceutical dosage formulation of claim 51, wherein administration to a mammal of a single dose, as compared to administration of an approximate equimolar dosage of semaglutide, exhibits a lower Cₘₐₓ at about 1 day, about 2 days, about 3 days, about 4 days, about 5 days, about 6 days or about 7 days following administration.

54. The pharmaceutical dosage formulation of claim 49, wherein the dosage is configured to administer to a human between about 0.1 to about 15 mg per week; optionally about 1 to about 7 mg per week; or optionally about 1 to 5 mg per week.

55. The pharmaceutical dosage formulation of claim 49 configured to be administered to the mammal once weekly for at least, or up to six weeks.

56. The pharmaceutical dosage formulation of claim 49, wherein the dosage is configured to reach a therapeutic dose in about four weeks or less following first weekly administration.

57. The pharmaceutical dosage formulation of claim 56, wherein the therapeutic dose exhibits a Cₘₐₓ of from about 10 to about 300 ng/ml, optionally a Cₘₐₓ less than 200 ng/ml; a Tₘₐₓ of from about 10 to about 36 hours; and/or, an AUC₀₋₁₆₈ of from about 1,000 to 100,000 h*ng/mL.

58. A method for inducing weight loss in a mammal, the method comprising administering pharmaceutical dosage formulation of claim 49 to a mammal, wherein the method reduces the incidence of one of more adverse events as compared to an agonist with unbalanced affinity for GLP-1R and GCGR, the adverse events being selected from nausea, vomiting, diarrhea, abdominal pain and constipation, upon administration to a mammal at a therapeutic dose.

59. The method of claim 58, wherein the pharmaceutical dosage is administered about weekly wherein an initial dose is the therapeutic dose.

60. The method of claim 58, wherein the pharmaceutical dosage is administered about weekly from about 2 weeks to about 8 weeks, or longer.
